(19) United States
(12) Patent Application Publication
(10) Pub. No.: US 2010/0160362 A1
(43) Pub. Date: Jun. 24, 2010

(54) SPIRO (FURO [3, 2-C] PYRIDINE-3-3' -INDOL) -2' (1'H)-ONE DERIVATIVES AND RELATED COMPOUNDS FOR THE TREATMENT OF SODIUM-CHANNEL MEDIATED DISEASES, SUCH AS PAIN

(75) Inventors: Jean-Jacques Cadieux, Burnaby (CA); Sultan Chowdhury, Surrey (CA); Jianmin Fu, Coquitlam (CA); Rajender Kamboj, Burnaby (CA); Tom Hsieh, Toronto (CA); Qi Jia, Burnaby (CA); Shifeng Liu, Coquitlam (CA); Jianyu Sun, San Mateo, CA (US)

Correspondence Address: Seed IP Law Group PLLC, 701 Fifth Avenue, Suite 5400, Seattle, WA, 98104, US

(73) Assignee: Xenon Pharmaceuticals Inc., Burnaby ,BC (CA)

(21) Appl. No.: 12/445,270

(22) PCT Filed: Oct. 12, 2007
(86) PCT No.: PCT/US2007/081244

§ 371 Date: Mar. 10, 2010

Related U.S. Application Data
(60) Provisional application No. 60/851,237, filed on Oct. 12, 2006.
(60) Provisional application No. 60/955,582, filed on Aug. 13, 2007.

(51) Int. Cl. A61K 31/438; C07D 491/20

(52) U.S. Cl. 514/278; 546/15

(57) ABSTRACT

This invention is directed to methods of using spiro-oxindole compounds of formula (I): wherein j, k, m, Q, X, R¹, R^{2a}, R^{2b}, R^{2c}, R_{2d} and R³ are as defined herein, as a stereoisomer, enantiomer, tautomer thereof or mixtures thereof; or a pharmaceutically acceptable salt, N-oxide, solvate or prodrug thereof, for the treatment and/or prevention of sodium channel-mediated diseases or conditions, such as pain.

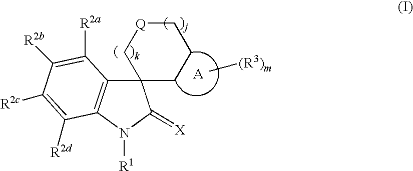

### FIELD OF THE INVENTION

The present invention is directed to spiro-oxindole compounds and pharmaceutical compositions comprising the compounds and methods of using the compounds and the pharmaceutical compositions in treating sodium channel-mediated diseases or conditions, such as pain, as well as other diseases and conditions associated with the mediation of sodium channels.

### BACKGROUND OF THE INVENTION

Voltage-gated sodium channels, transmembrane proteins that initiate action potentials in nerve, muscle and other electrically excitable cells, are a necessary component of normal sensation, emotions, thoughts and movements (Catterall, W. A., Nature (2001), Vol. 409, pp. 988-990). These channels consist of a highly processed alpha subunit that is associated with auxiliary beta subunits. The pore-forming alpha subunit is sufficient for channel function, but the kinetics and voltage dependence of channel gating are in part modified by the beta subunits (Goldin et al., Neuron (2000), Vol. 28, pp. 365-368). Each alpha-subunit contains four homologous domains, I to IV, each with six predicted transmembrane segments. The alpha-subunit of the sodium channel, forming the ion-conducting pore and containing the voltage sensors regulating sodium ion conduction has a relative molecular mass of 260,000. Electrophysiological recording, biochemical purification, and molecular cloning have identified ten different sodium channel alpha subunits and four beta subunits (Yu, F. H., et al., Sci. STKE (2004), 253; and Yu, F. H., et al., Neurosci. (2003), 20:7577-85).

The hallmarks of sodium channels include rapid activation and inactivation when the voltage across the plasma membrane of an excitable cell is depolarized (voltage-dependent gating), and efficient and selective conduction of sodium ions through conducting pores intrinsic to the structure of the protein (Sato, C., et al., Nature (2001), 409:1047-1051). At negative or hyperpolarized membrane potentials, sodium channels are closed. Following membrane depolarization, sodium channels open rapidly and then inactivate. Channels only conduct currents in the open state and, once inactivated, have to return to the resting state, favoured by membrane hyperpolarization, before they can reopen. Different sodium channel subtypes vary in the voltage range over which they activate and inactivate as well as their activation and inactivation kinetics.

The sodium channel family of proteins has been extensively studied and shown to be involved in a number of vital body functions. Research in this area has identified variants of the alpha subunits that result in major changes in channel function and activities, which can ultimately lead to major pathophysiological conditions. Implicit with function, this family of proteins are considered prime points of therapeutic intervention. Naᵥ1.1 and Naᵥ1.2 are highly expressed in the brain (Raymond, C. K., et al., J. Biol. Chem. (2004), 279(44):46234-41) and are vital to normal brain function. In humans, mutations in Naᵥ1.1 and Naᵥ1.2 result in severe epileptic states and in some cases mental decline (Rhodes, T. H., et al., Proc. Natl. Acad. Sci. USA (2004), 101(30):11147-52; Kamiya, K., et al., J. Biol. Chem. (2004), 24(11):2690-8; Pereira, S., et al., Neurology (2004), 63(1):191-2). As such both channels have been considered as validated targets for the treatment of epilepsy (see PCT Published Patent Publication No. WO 01/38564).

Naᵥ1.3 is broadly expressed throughout the body (Raymond, C. K., et al., op. cit.). It has been demonstrated to have its expression upregulated in the dorsal horn sensory neurons of rats after nervous system injury (Hains, B. D., et al., J. Neurosci. (2003), 23(26):8881-92). Many experts in the field have considered Naᵥ1.3 as a suitable target for pain therapeutics (Lai, J., et al., Curr. Opin. Neurobiol. (2003), (3):291-72003; Wood, J. N., et al., J. Neurobiol. (2004), 61(1):55-71; Chung, J. M., et al., Novartis Found Symp. (2004), 261:19-27; discussion 27-31, 47-54).

Naᵥ1.4 expression is essentially limited to muscle (Raymond, C. K., et al., op. cit.). Mutations in this gene have been shown to have profound effects on muscle function including paralysis, (Tamaoka A., Intern. Med. (2003), (9):769-70). Thus, this channel can be considered a target for the treatment of abnormal muscle contractility, spasm or paralysis.

The cardiac sodium channel, Naᵥ1.5, is expressed mainly in the heart ventricles and atria (Raymond, C. K., et al., op. cit.), and can be found in the sinovial node, ventricular node and possibly Purkinje cells. The rapid upstroke of the cardiac action potential and the rapid impulse conduction through cardiac tissue is due to the opening of Naᵥ1.5. As such, Naᵥ1.5 is central to the genesis of cardiac arrhythmias. Mutations in human Naᵥ1.5 result in multiple arrhythmic syndromes, including, for example, long QT3 (LQT3), Brugada syndrome (BS), an inherited cardiac conduction defect, sudden unexpected nocturnal death syndrome (SUNDS) and sudden infant death syndrome (SIDS) (Liu, H. et al., Am. J. Pharmacogenomics (2003), 3(3):173-9). Sodium channel blocker therapy has been used extensively in treating cardiac arrhythmias. The first antiarrhythmic drug, quinidine, discovered in 1914, is classified as a sodium channel blocker.

Naᵥ1.6 encodes an abundant, widely distributed voltage-gated sodium channel found throughout the central and peripheral nervous systems, clustered in the nodes of Ranvier of neural axons (Caldwell, J. H., et al., Proc. Natl. Acad. Sci. USA (2000), 97(10): 5616-20). Although no mutations in humans have been detected, Naᵥ1.6 is thought to play a role in the manifestation of the symptoms associated with multiple sclerosis and has been considered as a target for the treatment of this disease (Craner, M. J., et al., Proc. Natl. Acad. Sci. USA (2004), 101(21):8168-73).

Naᵥ1.7 was first cloned from the pheochromocytoma PC12 cell line (Toledo-Aral, J. J., et al., Proc. Natl. Acad. Sci. USA (1997), 94:1527-1532). Its presence at high levels in the growth cones of small-diameter neurons suggested that it could play a role in the transmission of nociceptive information. Although this has been challenged by experts in the field as Naᵥ1.7 is also expressed in neuroendocrine cells associated with the autonomic system (Klugbauer, N., et al., EMBO J. (1995), 14(6):1084-90) and as such has been implicated in autonomic processes. The implicit role in autonomic functions was demonstrated with the generation of Naᵥ1.7 null mutants; deleting Naᵥ1.7 in all sensory and sympathetic neurons resulted in a lethal perinatal phenotype. (Nassar, et al., Proc. Natl. Acad. Sci. USA (2004), 101(34):12706-11.). In contrast, by deleting the Naᵥ1.7 expression in a subset of sensory neurons that are predominantly nociceptive, a role in pain mechanisms, was demonstrated (Nassar, et al., op. cit.). Further support for Naᵥ1.7 blockers active in a subset of neurons is supported by the finding that two human heritable pain conditions, primary erythermalgia and familial rectal pain, have been shown to map to Naᵥ1.7 (Yang, Y., et al., J. Med. Genet. (2004), 41(3):171-4).

The expression of Naᵥ1.8 is essentially restricted to the DRG (Raymond, C. K., et al., op. cit.). There are no identified human mutations for Naᵥ1.8. However, Naᵥ1.8-null mutant mice were viable, fertile and normal in appearance. A pronounced analgesia to noxious mechanical stimuli, small deficits in noxious thermoreception and delayed development of inflammatory hyperalgesia suggested to the researchers that Naᵥ1.8 plays a major role in pain signalling (Akopian, A. N., et al., Nat. Neurosci. (1999), 2(6): 541-8). Blocking of this channel is widely accepted as a potential treatment for pain (Lai, J, et al., op. cit.; Wood, J. N., et al., op. cit.; Chung, J. M., et al., op. cit.). PCT Published Patent Application No. WO03/037274A2 describes pyrazole-amides and sulfonamides for the treatment of central or peripheral nervous system conditions, particularly pain and chronic pain by blocking sodium channels associated with the onset or reoccurrence of the indicated conditions. PCT Published Patent Application No. WO03/037890A2 describes piperidines for the treatment of central or peripheral nervous system conditions, particularly pain and chronic pain by blocking sodium channels associated with the onset or recurrence of the indicated conditions. The compounds, compositions and methods of these inventions are of particular use for treating neuropathic or inflammatory pain by the inhibition of ion flux through a channel that includes a PN3 (Naᵥ1.8) subunit.

The tetrodotoxin insensitive, peripheral sodium channel Naᵥ1.9, disclosed by Dib-Hajj, S. D., et al. (see Dib-Hajj, S. D., et al., Proc. Natl. Acad. Sci. USA (1998), 95(15):8963-8) was shown to reside solely in the dorsal root ganglia. It has been demonstrated that Naᵥ1.9 underlies neurotrophin (BDNF)-evoked depolarization and excitation, and is the only member of the voltage gated sodium channel superfamily to be shown to be ligand mediated (Blum, R., Kafitz, K. W., Konnerth, A., Nature (2002), 419 (6908):687-93). The limited pattern of expression of this channel has made it a candidate target for the treatment of pain (Lai, J, et al., op. cit.; Wood, J. N., et al., op. cit.; Chung, J. M. et al., op. cit.).

NaX is a putative sodium channel, which has not been shown to be voltage gated. In addition to expression in the lung, heart, dorsal root ganglia, and Schwann cells of the peripheral nervous system, NaX is found in neurons and ependymal cells in restricted areas of the CNS, particularly in the circumventricular organs, which are involved in body-fluid homeostasis (Watanabe, E., et al., J. Neurosci. (2000), 20(20):7743-51). NaX-null mice showed abnormal intakes of hypertonic saline under both water- and salt-depleted conditions. These findings suggest that the NaX plays an important role in the central sensing of body-fluid sodium level and regulation of salt intake behaviour. Its pattern of expression and function suggest it as a target for the treatment of cystic fibrosis and other related salt regulating maladies.

Studies with the sodium channel blocker tetrodotoxin (TTX) used to lower neuron activity in certain regions of the brain, indicate its potential use in the treatment of addiction. Drug-paired stimuli elicit drug craving and relapse in addicts and drug-seeking behavior in rats. The functional integrity of the basolateral amygdala (BLA) is necessary for reinstatement of cocaine-seeking behaviour elicited by cocaine-conditioned stimuli, but not by cocaine itself. BLA plays a similar role in reinstatement of heroin-seeking behavior. TTX-induced inactivation of the BLA on conditioned and heroin-primed reinstatement of extinguished heroin-seeking behaviour in a rat model (Fuchs, R. A. and See, R. E., Psychopharmacology (2002) 160(4):425-33).

This closely related family of proteins has long been recognised as targets for therapeutic intervention. Sodium channels are targeted by a diverse array of pharmacological agents. These include neurotoxins, antiarrhythmics, anticonvulsants and local anesthetics (Clare, J. J., et al., Drug Discovery Today (2000) 5:506-520). All of the current pharmacological agents that act on sodium channels have receptor sites on the alpha subunits. At least six distinct receptor sites for neurotoxins and one receptor site for local anesthetics and related drugs have been identified (Cestele, S. et al., Biochimie (2000), Vol. 82, pp. 883-892).

The small molecule sodium channel blockers or the local anesthetics and related antiepileptic and antiarrhythmic drugs, interact with overlapping receptor sites located in the inner cavity of the pore of the sodium channel (Catterall, W. A., Neuron (2000), 26:13-25). Amino acid residues in the S6 segments from at least three of the four domains contribute to this complex drug receptor site, with the IVS6 segment playing the dominant role. These regions are highly conserved and as such most sodium channel blockers known to date interact with similar potency with all channel subtypes. Nevertheless, it has been possible to produce sodium channel blockers with therapeutic selectivity and a sufficient therapeutic window for the treatment of epilepsy (e.g. lamotrignine, phenyloin and carbamazepine) and certain cardiac arrhythmias (e.g. lignocaine, tocamide and mexiletine). However, the potency and therapeutic index of these blockers is not optimal and have limited the usefulness of these compounds in a variety of therapeutic areas where a sodium channel blocker would be ideally suited.

### SUMMARY OF THE INVENTION

The present invention is directed to spiro-oxindole compounds and pharmaceutical compositions comprising the compounds and methods of using the compounds and the pharmaceutical compositions of the invention for the treatment and/or prevention of sodium channel-mediated diseases or conditions, such as pain. The present invention is also directed to methods of using the compounds and pharmaceutical compositions comprising the compounds for the treatment of other sodium channel-mediated diseases or conditions, including, but not limited to central nervous conditions such as epilepsy, anxiety, depression and bipolar disease; cardiovascular conditions such as arrhythmias, atrial fibrillation and ventricular fibrillation; neuromuscular conditions such as restless leg syndrome, essential tremour and muscle paralysis or tetanus; neuroprotection against stroke, glaucoma, neural trauma and multiple sclerosis; and channelopathies such as erythromyalgia and familial rectal pain syndrome. The present invention is also directed to methods of using the compounds of the invention and pharmaceutical compositions comprising the compound for the treatment and/or prevention of diseases or conditions, such as hypercholesterolemia, benign prostatic hyperplasia, pruritis, and cancer.

Accordingly, in one aspect this invention is directed to compounds of formula (I):

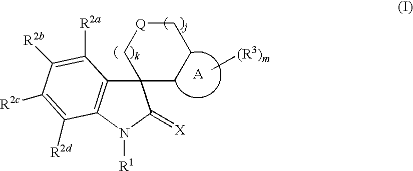

wherein:
j and k are each independently 0, 1, 2 or 3;
m is 0, 1, 2, or 4;
X is O or S;

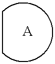

is a fused heterocyclyl ring or a fused heteroaryl ring;
Q is —C(R^{1a})₂—, —O—, —S(O)ₚ— (where p is 0, 1 or 2), —CF₂—, —OC(O)—, —C(O)O—, —C(O)N(R⁵)—, —N(R⁵)— or —N(R⁵)C(O)—;
each R^{1a} is hydrogen or —OR⁵;
or two R^{1a}'s, together with the carbon to which they are attached, form an oxo group;
R¹ is hydrogen, alkyl, alkenyl, alkynyl, haloalkyl, aryl, cycloalkyl, cycloalkylalkyl, heteroaryl, heterocyclyl, —R⁸—C(O)R⁵, —R⁸—C(O)OR⁵, —R⁸—C(O)N(R⁴)R⁵, —S(O)ₜ—R⁵ (where t is 1 or 2), —R⁹—S(O)ₚ—R⁵ (where p is 0, 1 or 2), —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
   R⁶ is hydrogen, alkyl, aryl or aralkyl; and
   R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
   or R⁶ and R⁷, together with the nitrogen to which they are attached, form a heterocyclyl or heteroaryl;
   and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by one or more substituents selected from the group consisting of —R⁸—OR⁵, —C(O)OR⁵, halo, haloalkyl, alkyl, nitro, cyano, aryl, aralkyl, heterocyclyl and heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
   each R¹⁰ is hydrogen, alkyl, aryl, aralkyl or heteroaryl;
   each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—N(R⁴)R⁵, —R⁹—OR⁵ or —R⁹—CN;
   R¹² is hydrogen, alkyl, aryl, aralkyl or —C(O)R⁵;
   and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁸—CN, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(═N—CN)N(R⁴)R⁵, wherein each p is independently 0, 1, or 2 and
   each t is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
or R^{2a} and R^{2b}, together with the carbon ring atoms to which they are directly attached, may form a fused ring selected from cycloalkyl, aryl, heterocyclyl and heteroaryl, and R^{2c} and R^{2d} are as defined above;
or R^{2b} and R^{2c}, together with the carbon ring atoms to which they are directly attached, may form a fused ring selected from cycloalkyl, aryl, heterocyclyl and heteroaryl, and R^{2a} and R^{2d} are as defined above;
or R^{2c} and R^{2d}, together with the carbon ring atoms to which they are directly attached, may form a fused ring selected from cycloalkyl, aryl, heterocyclyl and heteroaryl, and R^{2a} and R^{2b} are as defined above;
each R³ is independently selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁸—CN, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(N═C(R⁴)R⁵)N(R⁴)R⁵, wherein each p is independently 0, 1, or 2 and each t is independently 1 or 2;
   and wherein each cycloalkyl, cycloalkylalkyl, aryl, aralkyl, aralkenyl, heterocyclyl, heterocyclylalkyl, heteroaryl and heteroarylalkyl for R³ may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
each R⁴ and R⁵ is independently selected from group consisting of hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl and heteroarylalkyl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain;
as a stereoisomer, enantiomer, tautomer thereof or mixtures thereof;
or a pharmaceutically acceptable salt, N-oxide, solvate or prodrug thereof.

In another aspect, the invention provides methods for the treatment of pain in a mammal, preferably a human, wherein the methods comprise administering to the mammal in need thereof a therapeutically effective amount of a compound of the invention, as set forth above, as a stereoisomer, enantiomer, tautomer thereof or mixtures thereof; or a pharmaceutically acceptable salt, N-oxide, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, and a pharmaceutically acceptable excipient.

In another aspect, the present invention provides a method for treating or lessening the severity of a disease, condition, or disorder in a mammal where activation or hyperactivity of one or more of Naᵥ1.1, Naᵥ1.2, Naᵥ1.3, Naᵥ1.4, Naᵥ1.5, Naᵥ1.6, Naᵥ1.7, Naᵥ1.8, or Naᵥ1.9 is implicated in the disease, condition or disorder, wherein the method comprises administering to the mammal in need thereof a therapeutically effective amount of a compound of the invention, as set forth above, as a stereoisomer, enantiomer, tautomer thereof or mixtures thereof; or a pharmaceutically acceptable salt, N-oxide, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, and a pharmaceutically acceptable excipient.

In another aspect, the invention provides methods of treating a range of sodium channel-mediated diseases or conditions in a mammal, for example, pain associated with HIV, HIV treatment induced neuropathy, trigeminal neuralgia, post-herpetic neuralgia, eudynia, heat sensitivity, tosarcoidosis, irritable bowel syndrome, Crohns disease, pain associated with multiple sclerosis (MS), amyotrophic lateral sclerosis (ALS), diabetic neuropathy, peripheral neuropathy, arthritic, rheumatoid arthritis, osteoarthritis, atherosclerosis, paroxysmal dystonia, myasthenia syndromes, myotonia, malignant hyperthermia, cystic fibrosis, pseudoaldosteronism, rhabdomyolysis, hypothyroidism, bipolar depression, anxiety, schizophrenia, sodium channel toxin related illnesses, familial erythermalgia, primary erythermalgia, familial rectal pain, cancer, epilepsy, partial and general tonic seizures, restless leg syndrome, arrhythmias, fibromyalgia, neuroprotection under ischaemic conditions caused by stroke, glaucoma or neural trauma, tachy-arrhythmias, atrial fibrillation and ventricular fibrillation, wherein the methods comprise administering to the mammal in need thereof, preferably a human, a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, and a pharmaceutically acceptable excipient.

In another aspect, the invention provides methods of treating a range of sodium channel-mediated diseases or conditions in a mammal, preferably a human, by the inhibition of ion flux through a voltage-dependent sodium channel in the mammal, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, and a pharmaceutically acceptable excipient.

In another aspect, the invention provides methods of treating or preventing hypercholesterolemia in a mammal, preferably a human, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, and a pharmaceutically acceptable excipient.

In another aspect, the invention provides methods of treating or preventing benign prostatic hyperplasia in a mammal, preferably a human, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, and a pharmaceutically acceptable excipient.

In another aspect, the invention provides methods of treating or preventing pruritis in a mammal, preferably a human, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, and a pharmaceutically acceptable excipient.

In another aspect, the invention provides methods of treating or preventing cancer in a mammal, preferably a human, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, and a pharmaceutically acceptable excipient.

In another aspect, the invention provides pharmaceutical therapy in combination with one or more other compounds of the invention or one or more other accepted therapies or as any combination thereof to increase the potency of an existing or future drug therapy or to decrease the adverse events associated with the accepted therapy. In one embodiment, the present invention relates to a pharmaceutical composition combining compounds of the present invention with established or future therapies for the indications listed in the invention.

In another aspect, this invention is directed to the use of the compounds of the invention, as set forth above, as a stereoisomer, enantiomer, tautomer thereof or mixtures thereof, or a pharmaceutically acceptable salt, N-oxide, solvate or prodrug thereof, or the use of a pharmaceutical composition comprising a pharmaceutically acceptable excipient and a compound of the invention, as set forth above, as a stereoisomer, enantiomer, tautomer thereof or mixtures thereof, or a pharmaceutically acceptable salt, N-oxide, solvate or prodrug thereof, in the preparation of a medicament for the treatment of pain in a mammal.

In another aspect, this invention is directed to the use of the compounds of the invention, as set forth above, as a stereoisomer, enantiomer, tautomer thereof or mixtures thereof, or a pharmaceutically acceptable salt, N-oxide, solvate or prodrug thereof, or the use of a pharmaceutical composition comprising a pharmaceutically acceptable excipient and a compound of the invention, as set forth above, as a stereoisomer, enantiomer, tautomer thereof or mixtures thereof, or a pharmaceutically acceptable salt, N-oxide, solvate or prodrug thereof, in the preparation of a medicament for the treatment of sodium channel-mediated disease or condition in a mammal.

### DETAILED DESCRIPTION OF THE INVENTION

### Definitions

Certain chemical groups named herein may be preceded by a shorthand notation indicating the total number of carbon atoms that are to be found in the indicated chemical group. For example; C₇-C₁₂alkyl describes an alkyl group, as defined below, having a total of 7 to 12 carbon atoms, and C₄-C₁₂cycloalkylalkyl describes a cycloalkylalkyl group, as defined below, having a total of 4 to 12 carbon atoms. The total number of carbons in the shorthand notation does not include carbons that may exist in substituents of the group described.

In addition to the foregoing, as used in the specification and appended claims, unless specified to the contrary, the following terms have the meaning indicated:

“Amino” refers to the —NH₂ radical.

“Cyano” refers to the —CN radical.

“Hydroxy” refers to the —OH radical.

“Imino” refers to the ═NH substituent.

“Nitro” refers to the —NO₂ radical.

“N-oxide” refers to the functional group N→O. N-oxides are prepared by the oxidation of a tertiary amine (including aromatic amines such as those found in pyridine).

“Oxo” refers to the ═O substituent.

“Thioxo” refers to the ═S substituent.

“Trifluoromethyl” refers to the —CF₃ radical.

“Alkyl” refers to a straight or branched hydrocarbon chain radical consisting solely of carbon and hydrogen atoms, containing no unsaturation, having from one to twelve carbon atoms, preferably one to eight carbon atoms or one to six carbon atoms, and which is attached to the rest of the molecule by a single bond, e.g., methyl, ethyl, n-propyl, 1-methylethyl(iso-propyl), n-butyl, n-pentyl, 1,1-dimethylethyl(t-butyl), 3-methylhexyl, 2-methylhexyl, and the like. Unless stated otherwise specifically in the specification, an alkyl group may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkenyl” refers to a straight or branched hydrocarbon chain radical group consisting solely of carbon and hydrogen atoms, containing at least one double bond, having from two to twelve carbon atoms, preferably two to eight carbon atoms and which is attached to the rest of the molecule by a single bond, e.g., ethenyl, prop-1-enyl, but-1-enyl, pent-1-enyl, penta-1,4-dienyl, and the like. Unless stated otherwise specifically in the specification, an alkenyl group may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkynyl” refers to a straight or branched hydrocarbon chain radical group comprising solely of carbon and hydrogen atoms, containing at least one triple bond, optionally containing at least one double bond, having from two to twelve carbon atoms, preferably two to eight carbon atoms and which is attached to the rest of the molecule by a single bond, for example, ethynyl, propynyl, butynyl, pentynyl, hexynyl, and the like. Unless stated otherwise specifically in the specification, an alkynyl group may be optionally substituted by one or more of the following substituents: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one double bond and having from two to twelve carbon atoms, e.g., ethenylene, propenylene, n-butenylene, and the like. The alkenylene chain is attached to the rest of the molecule through a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkynylene” or “alkynylene chain” refers to a straight or branched divalent hydrocarbon chain linking the rest of the molecule to a radical group, consisting solely of carbon and hydrogen, containing at least one triple bond and having from two to twelve carbon atoms, e.g., propynylene, n-butynylene, and the like. The alkynylene chain is attached to the rest of the molecule through a single bond and to the radical group through a double bond or a single bond. The points of attachment of the alkynylene chain to the rest of the molecule and to the radical group can be through one carbon or any two carbons within the chain. Unless stated otherwise specifically in the specification, an alkynylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 to 2), —S(O)ₜOR¹⁶ (where t is 1 to 2), —S(O)ₚR¹⁶ (where p is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Alkoxy” refers to a radical of the formula —ORₐ where Rₐ is an alkyl radical as defined above containing one to twelve carbon atoms. The alkyl part of the alkoxy radical may be optionally substituted as defined above for an alkyl radical.

“Alkoxyalkyl” refers to a radical of the formula —R_{b}—O—Rₐ where R_{b} is an alkylene chain as defined above and Rₐ is an alkyl radical as defined above. The oxygen atom may be bonded to any carbon in the alkylene chain and in the alkyl radical. The alkyl part of the alkoxyalkyl radical may be optionally substituted as defined above for an alkyl group. The alkylene chain part of the alkoxyalkyl radical may be optionally substituted as defined above for an alkylene chain.

“Aryl” refers to a hydrocarbon ring system radical comprising hydrogen, 6 to 18 carbon atoms and at least one aromatic ring. For purposes of this invention, the aryl radical may be a monocyclic, bicyclic, tricyclic or tetracyclic ring system, which may included fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals optionally substituted by one or more substituents independently selected from the group consisting of alkyl, alkenyl, halo, haloalkyl, haloalkenyl, cyano, nitro, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 to 2), —R¹⁵—N═C(OR¹⁴)R¹⁶, —R¹⁵—S(O)ₜOR¹⁶ (where t is 1 to 2), —R¹⁵—S(O)ₚR¹⁶ (where p is 0 to 2), and —R¹⁵—S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Aralkyl” refers to a radical of the formula —R_{b}—R_{c} where R_{b} is an alkylene chain as defined above and Rₑ is one or more aryl radicals as defined above, for example, benzyl, diphenylmethyl and the like. The alkylene chain part of the aralkyl radical may be optionally substituted as described above for an alkylene chain. The aryl part of the aralkyl radical may be optionally substituted as described above for an aryl group.

“Aralkenyl” refers to a radical of the formula —R_{d}—R_{c} where R_{d} is an alkenylene chain as defined above and R_{c} is one or more aryl radicals as defined above. The aryl part of the aralkenyl radical may be optionally substituted as described above for an aryl group. The alkenylene chain part of the aralkenyl radical may be optionally substituted as defined above for an alkenylene group.

“Aralkynyl” refers to a radical of the formula —RₑR_{c} where Rₑ is an alkynylene chain as defined above and R_{c} is one or more aryl radicals as defined above. The aryl part of the aralkynyl radical may be optionally substituted as described above for an aryl group. The alkynylene chain part of the aralkynyl radical may be optionally substituted as defined above for an alkynylene chain.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptly, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, the term “cycloalkyl” is meant to include cycloalkyl radicals which are optionally substituted by one or more substituents independently selected from the group consisting of alkyl, alkenyl, halo, haloalkyl, haloalkenyl, cyano, nitro, oxo, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 to 2), —R¹⁵—N═C(OR¹⁴)R¹⁶, —R¹⁵—S(O)ₜOR¹⁶ (where t is 1 to 2), —R¹⁵—S(O)ₚR¹⁶ (where p is 0 to 2), and —R¹⁵—S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl or heteroarylalkyl.

“Cycloalkylalkyl” refers to a radical of the formula —R_{b}R_{g} where R_{b} is an alkylene chain as defined above and R_{g} is a cycloalkyl radical as defined above. The alkylene chain and the cycloalkyl radical may be optionally substituted as defined above.

“Cycloalkylalkenyl” refers to a radical of the formula —R_{d}R_{g} where R_{d} is an alkenylene chain as defined above and R_{g} is a cycloalkyl radical as defined above. The alkenylene chain and the cycloalkyl radical may be optionally substituted as defined above.

“Cycloalkylalkynyl” refers to a radical of the formula —RₑR_{g} where Rₑ is an alkynylene radical as defined above and R_{g} is a cycloalkyl radical as defined above. The alkynylene chain and the cycloalkyl radical may be optionally substituted as defined above.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. The alkyl part of the haloalkyl radical may be optionally substituted as defined above for an alkyl group.

“Haloalkenyl” refers to an alkenyl radical, as defined above, that is substituted by one or more halo radicals, as defined above. The alkenyl part of the haloalkyl radical may be optionally substituted as defined above for an alkenyl group.

“Haloalkynyl” refers to an alkynyl radical, as defined above, that is substituted by one or more halo radicals, as defined above. The alkynyl part of the haloalkyl radical may be optionally substituted as defined above for an alkynyl group.

“Heterocyclyl” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above which are optionally substituted by one or more substituents selected from the group consisting of alkyl, alkenyl, halo, haloalkyl, haloalkenyl, cyano, oxo, thioxo, nitro, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 to 2), —R¹⁵—N═C(OR¹⁴)R¹⁴, —R¹⁵—S(O)ₜOR¹⁶ (where t is 1 to 2), —R¹⁵—S(O)ₚR¹⁶ (where p is 0 to 2), and —R¹⁵—S(O)ₜN(R¹⁴)₂ (where t is 1 to 2) where each R¹⁴ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, alkenyl, haloalkyl, cycloalkyl, cycloalkylalkyl, aryl, aralkyl, heterocyclyl, heterocyclylalkyl, heteroaryl or heteroarylalkyl.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical may be optionally substituted as described above for heterocyclyl radicals.

“Heterocyclylalkyl” refers to a radical of the formula —R_{b}Rₕ where R_{b} is an alkylene chain as defined above and Rₕ is a heterocyclyl radical as defined above, and if the heterocyclyl is a nitrogen-containing heterocyclyl, the heterocyclyl may be attached to the alkyl radical at the nitrogen atom. The alkylene chain of the heterocyclylalkyl radical may be optionally substituted as defined above for an alkyene chain. The heterocyclyl part of the heterocyclylalkyl radical may be optionally substituted as defined above for a heterocyclyl group.

“Heterocyclylalkenyl” refers to a radical of the formula —R_{d}Rₕ where R_{d} is an alkenylene chain as defined above and Rₕ is a heterocyclyl radical as defined above, and if the heterocyclyl is a nitrogen-containing heterocyclyl, the heterocyclyl may be attached to the alkenylene chain at the nitrogen atom. The alkenylene chain of the heterocyclylalkenyl radical may be optionally substituted as defined above for an alkenylene chain. The heterocyclyl part of the heterocyclylalkenyl radical may be optionally substituted as defined above for a heterocyclyl group.

“Heterocyclylalkynyl” refers to a radical of the formula —RₑRₕ where Rₑ is an alkynylene chain as defined above and Rₕ is a heterocyclyl radical as defined above, and if the heterocyclyl is a nitrogen-containing heterocyclyl, the heterocyclyl may be attached to the alkynyl radical at the nitrogen atom. The alkynylene chain part of the heterocyclylalkynyl radical may be optionally substituted as defined above for an alkynylene chain. The heterocyclyl part of the heterocyclylalkynyl radical may be optionally substituted as defined above for a heterocyclyl group.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl(benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrrolyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, the term “heteroaryl” is meant to include heteroaryl radicals as defined above which are optionally substituted by one or more substituents selected from the group consisting of alkyl, alkenyl, alkoxy, halo, haloalkyl, haloalkenyl, cyano, oxo, thioxo, nitro, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 to 2), —R¹⁵—N═C(OR¹⁴)R¹⁴, —R¹⁵—S(O)ₜOR¹⁶ (where t is 1 to 2), —R¹⁵—S(O)ₚR¹⁶ (where p is 0 to 2), and —R¹⁵—S(O)ₜN(R¹⁴)₂ (where t is 1 to 2) where each R¹⁴ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, alkenyl, haloalkyl, cycloalkyl, cycloalkylalkyl, aryl, aralkyl, heterocyclyl, heterocyclylalkyl, heteroaryl or heteroarylalkyl.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical may be optionally substituted as described above for heteroaryl radicals.

“Heteroarylalkyl” refers to a radical of the formula —R_{b}Rᵢ, where R_{b} is an alkylene chain as defined above and Rᵢ is a heteroaryl radical as defined above. The heteroaryl part of the heteroarylalkyl radical may be optionally substituted as defined above for a heteroaryl group. The alkylene chain part of the heteroarylalkyl radical may be optionally substituted as defined above for an alkylene chain.

“Heteroarylalkenyl” refers to a radical of the formula —R_{d}Rᵢ, where R_{d} is an alkenylene chain as defined above and Rᵢ is a heteroaryl radical as defined above. The heteroaryl part of the heteroarylalkenyl radical may be optionally substituted as defined above for a heteroaryl group. The alkenylene chain part of the heteroarylalkenyl radical may be optionally substituted as defined above for an alkenylene chain.

“Heteroarylalkynyl” refers to a radical of the formula —RₑRᵢ, where Rₑ is an alkynylene chain as defined above and Rᵢ is a heteroaryl radical as defined above. The heteroaryl part of the heteroarylalkynyl radical may be optionally substituted as defined above for a heteroaryl group. The alkynylene chain part of the heteroarylalkynyl radical may be optionally substituted as defined above for an alkynylene chain.

“Hydroxyalkyl” refers to an alkyl radical, as defined above, substituted by one or more hydroxy groups.

“Analgesia” refers to an absence of pain in response to a stimulus that would normally be painful.

“Allodynia” refers to a condition in which a normally innocuous sensation, such as pressure or light touch, is perceived as being extremely painful.

“Prodrugs” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., “Pro-drugs as Novel Delivery Systems,” A.C.S. Symposium Series, Vol. 14, and in Bioreversible Carriers in Drug Design, Ed. Edward B. Roche, American Pharmaceutical Association and Pergamon Press, 1987, both of which are incorporated in full by reference herein.

The term “prodrug” is also meant to include any covalently bonded carriers, which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of the invention and the like.

The invention disclosed herein is also meant to encompass all pharmaceutically acceptable compounds of formula (I) being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabelled compounds could be useful to help determine or measure the effectiveness of the compounds, by characterizing, for example, the site or mode of action on the sodium channels, or binding affinity to pharmacologically important site of action on the sodium channels. Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Preparations and Examples as set out below using an appropriate isotopically-labeled reagent in place of the non-labeled reagent previously employed.

The invention disclosed herein is also meant to encompass the in vivo metabolic products of the disclosed compounds. Such products may result from, for example, the oxidation, reducation, hydrolysis, amidation, esterification, and the like of the administered compound, primarily due to enzymatic processes. Accordingly, the invention includes compounds produced by a process comprising contacting a compound of this invention with a mammal for a period of time sufficient to yield a metabolic product thereof. Such products are typically are identified by administering a radiolabelled compound of the invention in a detectable dose to an animal, such as rat, mouse, guinea pig, monkey, or to human, allowing sufficient time for metabolism to occur, and isolating its coversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets, (e.g. cats, dogs, swine, cattle, sheep, goats, horses, rabbits), and non-domestic animals such as wildlife and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution. When a functional group is described as “optionally substituted,” and in turn, substitutents on the functional group are also “optionally substituted” and so on, for the purposes of this invention, such iterations are limited to five.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“Therapeutically effective amount” refers to that amount of a compound of the invention which, when administered to a mammal, preferably a human, is sufficient to effect treatment, as defined below, of a sodium channel-mediated disease or condition in the mammal, preferably a human. The amount of a compound of the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:

preventing the disease or condition from occurring in a mammal, in particular, when such mammal is predisposed to the condition but has not yet been diagnosed as having it;

(ii) inhibiting the disease or condition, i.e., arresting its development;

(iii) relieving the disease or condition, i.e., causing regression of the disease or condition; or

(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the invention, or their pharmaceutically acceptable salts may contain one or more asymmetric centres and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallisation. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present invention includes tautomers of any said compounds.

Also within the scope of the invention are intermediate compounds of formula (I) and all polymorphs of the aforementioned species and crystal habits thereof.

The chemical naming protocol and structure diagrams used herein are a modified form of the I.U.P.A.C. nomenclature system, using the ACD/Name Version 9.07 software program and/or ChemDraw Version 10.0 software naming program (CambridgeSoft), wherein the compounds of the invention are named herein as derivatives of the central core structure, e.g., the imidazopyridine structure. For complex chemical names employed herein, a substituent group is named before the group to which it attaches. For example, cyclopropylethyl comprises an ethyl backbone with cyclopropyl substituent. Except as described below, all bonds are identified in the chemical structure diagrams herein, except for some carbon atoms, which are assumed to be bonded to sufficient hydrogen atoms to complete the valency.

With respect to the chemical structure of compounds of formula (Ia), as set forth below in the Embodiments of the Invention, the following structure:

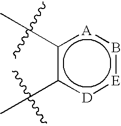

is intended to indicate an aromatic ring.

Thus, for example, a compound of formula (Ia) wherein j is 0; k is 1; m is 0; Q is —O—; R¹ is n-pentyl; R^{2a}, R^{2b}, R^{2c}, R^{2d} and R³ are each hydrogen; A, B, and E are each C(H); and D is N; i.e., a compound of the following formula:

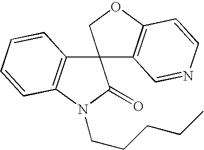

is named herein as 1′-pentylspiro[furo[3,2-c]pyridine-3,3′-indol]-2′(1′H)-one.

### EMBODIMENTS OF THE INVENTION

Of the various aspects of the invention set forth above in the Summary of the Invention, certain embodiments are preferred.

Of the compounds of formula (I), as set forth above in the Summary of the Invention, one embodiment is a compound of formula (I) wherein

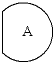

is a fused heterocyclyl ring.

Of the compounds of formula (I), as set forth above in the Summary of the Invention, another embodiment is a compound of formula (I) wherein X is O and

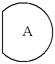

is a fused heteroaryl ring.

Of this embodiment, one embodiment is a compound of formula (I) which is a compound of formula (Ia):

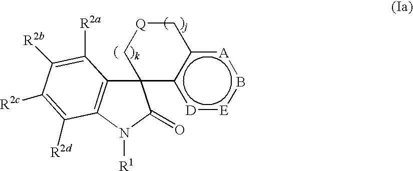

wherein:
j and k are each independently 0, 1, 2 or 3;
Q is —C(R^{1a})H—, —C(O)—, —O—, —S—, —N(R⁵)—, —CF₂—, —C(O)O—, —C(O)N(R⁵)— or —N(R⁵)C(O)—;
A is C(R^{3a}), N or N→O;
B is C(R^{3b}), N or N→O;
D is C(R^{3d}), N or N→O;
E is C(R^{3e}), N or N→O; provided that at least one of A, B, D and E is N or N→O and that no more than two of A, B, C and D are N or N→O at the same time;
or A is C(R^{3a}), B is C(R^{3b}), E is N(H) and D is C(O);
or A is C(R^{3a}), B is C(R^{3b}), D is N(H) and E is C(O);
R^{1a} is hydrogen or —OR⁵;
R¹ is hydrogen, alkyl, alkenyl, alkynyl, haloalkyl, aryl, cycloalkyl, cycloalkylalkyl, heteroaryl, heterocyclyl, —R⁸—OR⁵, —R⁸—CN, —R⁹—P(O)(OR⁵)₂, or —R⁹—O—R⁹—OR⁵;
or R¹ is aralkyl substituted by —C(O)N(R⁶)R⁷ where:
   R⁶ is hydrogen, alkyl, aryl or aralkyl; and
   R⁷ is hydrogen, alkyl, haloalkyl, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
   or R⁶ and R⁷, together with the nitrogen to which they are attached, form a heterocyclyl or heteroaryl;
   and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
   each R¹⁵ is hydrogen, alkyl, aryl or aralkyl;
   each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—OR⁵ or —R⁹—CN;
   R¹² is hydrogen, alkyl, aryl, aralkyl or —C(O)R⁵;
   and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁵, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(═N—CN)N(R⁴)R⁵, wherein each m is independently 0, 1, or 2 and each n is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{2a} and R^{2b}, R^{2b} and R^{2c}, or R^{2c} and R^{2d}, together with the carbon ring atoms to which they are directly attached, may form a fused ring selected from cycloalkyl, aryl, heterocyclyl and heteroaryl;
R^{3a}, R^{3b}, R^{3e} and R^{3d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(N═C(R⁴)R⁵)N(R⁴)R⁵, wherein each m is independently 0, 1, or 2 and each n is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3b}, R^{3c} and R^{3d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3a} and R^{3b}, or R^{3b} and R^{3e}, or R^{3e} and R^{3d}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain;
as a stereoisomer, enantiomer, tautomer thereof or mixtures thereof;
or a pharmaceutically acceptable salt, solvate or prodrug thereof.

Another embodiment is a compound of formula (I) which is a compound of formula (Ia), as set forth above, wherein:
j and k are each independently 0, 1, 2 or 3;
Q is —C(R^{1a})H—, —C(O)—, —O—, —S—, —N(R⁵)—, —CF₂—, —C(O)O—, —C(O)N(R⁵)— or —N(R⁵)C(O)—;
A is C(R^{3a});
B is C(R^{3b}) or N;
E is C(R^{3e});
D is C(R^{3d}) or N, provided that at least one of B and D is N;
R^{1a} is hydrogen or —OR⁵;
R¹ is hydrogen, alkyl, alkenyl, alkynyl, haloalkyl, aryl, cycloalkyl, cycloalkylalkyl, heteroaryl, heterocyclyl, —R⁸—OR⁵, —R⁸—CN, —R⁹—P(O)(OR⁵)₂, or —R⁹—O—R⁹—OR⁵;
or R¹ is aralkyl substituted by —C(O)N(R⁶)R⁷ where:
   R⁶ is hydrogen, alkyl, aryl or aralkyl; and
   R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
   or R⁶ and R⁷, together with the nitrogen to which they are attached, form a heterocyclyl or heteroaryl;
   and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
   each R¹⁰ is hydrogen, alkyl, aryl or aralkyl;
   each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—OR⁵ or —R⁹—CN;
   R¹² is hydrogen, alkyl, aryl, aralkyl or —C(O)R⁵;
   and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(═N—CN)N(R⁴)R⁵, wherein each m is independently 0, 1, or 2 and each n is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{2a} and R^{2b}, or R^{2b} and R^{2c}, or R^{2c} and R^{2d}, together with the carbon ring atoms to which they are directly attached, may form a fused ring selected from cycloalkyl, aryl, heterocyclyl and heteroaryl;
R^{3a}, R^{3b}, R^{3e} and R^{3d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(N═C(R⁴)R⁵)N(R⁴)R⁵, wherein each m is independently 0, 1, or 2 and each n is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3b}, R^{3e} and R^{3d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3a} and R^{3b}, or R^{3b} and R^{3e}, or R^{3e} and R^{3d}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

Another embodiment is a compound of formula (I) which is a compound of formula (Ia), as set forth above, wherein:
j and k are each independently 0, 1, 2 or 3;
Q is —C(R^{1a})H—, —C(O)—, —O—, —S—, —N(R⁵)—, —CF₂—, —C(O)O—, —C(O)N(R⁵)— or —N(R⁵)C(O)—;
A is C(R^{3a});
B is C(R^{3b});
E is C(R^{3e});
D is N;
R^{1a} is hydrogen or —OR⁵;
R¹ is hydrogen, alkyl, alkenyl, alkynyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
   R⁶ is hydrogen, alkyl, aryl or aralkyl; and
   R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
   or R⁶ and R⁷, together with the nitrogen to which they are attached, form a heterocyclyl or heteroaryl;
   and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
   each R¹⁰ is hydrogen, alkyl, aryl or aralkyl;
   each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—OR⁵ or —R⁹—CN;
   R¹² is hydrogen, alkyl, aryl, aralkyl or —C(O)R⁵;
   and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵, and —N(R⁵)C(═N—CN)N(R⁴)R⁵, wherein each m is independently 0, 1, or 2 and each n is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{2a} and R^{2b}, or R^{2b} and R^{2c}, or R^{2c} and R^{2d}, together with the carbon ring atoms to which they are directly attached, may form a fused ring selected from cycloalkyl, aryl, heterocyclyl and heteroaryl;
R^{3a}, R^{3b}, R^{3e} and R^{3d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(N═C(R⁴)R⁵)N(R⁴)R⁵, wherein each m is independently 0, 1, or 2 and each n is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3b}, R^{3e} and R^{3d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3a} and R^{3b}, or R^{3b} and R^{3e}, or R^{3e} and R^{3d}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

Another embodiment is a compound of formula (I) which is a compound of formula (Ia), as set forth above, wherein:
j is 0 and k is 1;
Q is —C(R^{1a})H—, —O—, —S—, or —N(R⁵)—;
A is C(R^{3a});
B is C(R^{3b});
E is C(R^{3e});
D is N;
R^{1a} is hydrogen or —OR⁵;
R¹ is hydrogen, alkyl, alkenyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is —R⁹—C(O)N(R¹²)R¹¹ where:
   R¹¹ is hydrogen, alkyl, aryl or aralkyl;
   R¹² is hydrogen, alkyl, aryl, aralkyl or —C(O)R⁵;
   and wherein each aryl or aralkyl groups for R¹¹ may be optionally substituted by one or more substituents selected from the group consisting of alkyl, cycloalkyl, aryl, aralkyl, halo and haloalkyl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkoxy, halo, haloalkyl, haloalkenyl, haloalkoxy, cycloalkyl, cycloalkylalkyl, aryl, aralkyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴ and —R⁸—C(O)N(R⁴)R⁵;
   and wherein each of the cycloalkyl, cycloalkylalkyl, aryl, aralkyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein m is 0, 1, or 2 and n is 1 or 2;
R^{3a}, R^{3b} and R^{3e} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴,
   wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3b} and R^{3e} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3a} and R^{3b} or R^{3b} and R^{3e}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain; and
each R⁹ is a straight or branched alkylene chain.

Another embodiment is a compound of formula (I) which is a compound of formula (Ia), as set forth above, wherein:
j is 0 and k is 1;
Q is —O—;
A is C(R^{3a});
B is C(R^{3b});
E is C(R^{3e});
D is N;
R¹ is hydrogen, alkyl, alkenyl, haloalkyl, —R⁸—OR⁵ or —R⁸—CN;
or R¹ is —R⁹—C(O)N(R¹²)R¹¹ where:
   R¹¹ is hydrogen, alkyl, aryl or aralkyl;
   R¹² is hydrogen, alkyl, aryl, aralkyl or —C(O)R⁵;
   and wherein each aryl or aralkyl groups for R¹¹ may be optionally substituted by one or more substituents selected from the group consisting of alkyl, cycloalkyl, aryl, aralkyl, halo and haloalkyl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkoxy, halo, haloalkyl, heteroaryl, —R⁸—OR⁵, —R⁸—N(R⁴)R⁵, —R⁸—C(O)R⁴, —R⁸—C(O)OR⁴ and —R⁸—C(O)N(R⁴)R⁵;
R^{3a}, R^{3b} and R^{3e} are each independently selected from the group consisting of hydrogen, alkyl, alkoxy, halo, haloalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴;
or R^{3a} and R^{3b} or R^{3b} and R^{3e}, together with the carbon ring atoms to which they are directly attached, may form a fused heterocyclyl ring;
each R⁴ and R⁵ is independently selected from group consisting of hydrogen, alkyl, haloalkyl, alkoxyalkyl, cycloalkyl, cycloalkylalkyl, aryl, aralkyl, heterocyclyl and heteroaryl; and
each R⁸ is a direct bond or a straight or branched alkylene chain.

Another embodiment is a compound of formula (I) which is a compound of formula (Ia), as set forth above, wherein:
j is 0 and k is 1;
Q is —O—;
A is C(R^{3a});
B is C(R^{3b});
E is C(R^{3e});
D is N;
R¹ is hydrogen or heteroarylalkyl where the heteroarylalkyl group is optionally substituted by one or more substituents selected from the group consisting of alkyl, halo, haloalkyl, —R⁸—OR⁵ and —R⁸—C(O)OR⁵;
R^{2a}, R^{2b}, R^{2c} and R^{2d} are each independently selected from the group consisting of hydrogen, alkyl, halo, haloalkyl and heteroaryl;
R^{3a}, R^{3b} and R^{3e} are each independently selected from the group consisting of hydrogen, alkyl, alkoxy, halo, haloalkyl and —R⁸—OR⁵;
or R^{3a} and R^{3b} or R^{3b} and R^{3e}, together with the carbon ring atoms to which they are directly attached, may form a fused ring selected from heterocyclyl;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

Another embodiment is a compound of formula (I) which is a compound of formula (Ia), as set forth above, wherein:
j is 0 and k is 1;
Q is —O—;
A is C(R^{3a});
B is C(R^{3b});
E is C(R^{3e});
D is N;
R¹ is hydrogen;
R^{2a}, R^{2b}, R^{2c} and R^{2d} are each independently selected from the group consisting of hydrogen, alkyl, halo and haloalkyl;
R^{3a}, R^{3b} and R^{3e} are each independently selected from the group consisting of hydrogen, alkyl, alkoxy, halo, haloalkyl, and —R⁸—OR⁵;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

Another embodiment is a compound of formula (Ia) selected from the group consisting of:
4′-bromo-5-methoxyspiro[furo[3,2-b]pyridine-3,3′-indol]-2′(1′H)-one; and
5-methoxyspiro[furo[3,2-b]pyridine-3,3′-indol]-2′(1′H)-one.

Another embodiment is a compound of formula (I) which is a compound of formula (Ia), as set forth above, wherein:
j is 0 and k is 1;
Q is —O—;
A is C(R^{3a});
B is C(R^{3b});
E is C(R^{3e});
D is N;
R¹ is heteroarylalkyl where the heteroarylalkyl group is optionally substituted by one or more substituents selected from the group consisting of alkyl, halo, haloalkyl, —R⁸—OR⁵ and —R⁸—C(O)OR⁵;
R^{2a}, R^{2b}, R^{2c} and R^{2d} are each independently selected from the group consisting of hydrogen, alkyl, halo, haloalkyl and heteroaryl;
R^{3a}, R^{3b} and R^{3e} are each independently selected from the group consisting of hydrogen, alkyl, alkoxy, halo, haloalkyl and —R⁸—OR⁵;
or R^{3a} and R^{3b} or R^{3b} and R^{3e}, together with the carbon ring atoms to which they are directly attached, may form a fused ring selected from heterocyclyl;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

Another embodiment is a compound of formula (Ia) selected from the group consisting of:
4′-bromo-5-methoxy-1′-{[5-(trifluoromethyl)furan-2-yl]methyl}spiro[furo[3,2-b]pyridine-3,3′-indol]-2′(1′H)-one;
5-methoxy-1′-{[5-(trifluoromethyl)furan-2-yl]methyl}spiro[furo[3,2-b]pyridine-3,3′-indol]-2′(1′H)-one;
4′-furan-3-yl-5-methoxy-1′-{[5-(trifluoromethyl)furan-2-yl]methyl}spiro[furo[3,2-b]pyridine-3,3′-indol]-2′(1′H)-one;
1′-{[5-(trifluoromethyl)furan-2-yl]methyl}spiro[1,3-dioxolo[4,5-b]furo[2,3-e]pyridine-5,3′-indol]-2′(1′H)-one;
5′-fluoro-5-methoxy-1′-{[5-(trifluoromethyl)furan-2-yl]methyl}spiro[furo[3,2-b]pyridine-3,3′-indol]-2′(1′H)-one;
4′-chloro-5-methoxy-1′-{[5-(trifluoromethyl)furan-2-yl]methyl}spiro[furo[3,2-b]pyridine-3,3′-indol]-2′(1′H)-one;
1′-[(5-chloro-1-methyl-1H-imidazol-2-yl)methyl]-5-methoxyspiro[furo[3,2-b]pyridine-3,3′-indol]-2′(1′H)-one;
5-methoxy-1′-(pyridin-2-ylmethyl)spiro[furo[3,2-b]pyridine-3,3′-indol]-2′(1′H)-one;
4′-bromo-1′-[(2-isopropyl-1,3-thiazol-5-yl)methyl]-5-methoxyspiro[furo[3,2-b]pyridine-3,3′-indol]-2′(1′14)-one;
1′-[(5-chlorothiophen-2-yl)methyl]-5-methoxyspiro[furo[3,2-b]pyridine-3,3′-indol]-2′(1′H)-one; and
5-methoxy-1′-{[2-(1-methylethyl)-1,3-thiazol-4-yl]methyl}spiro[furo[3,2-b]pyridine-3,3′-indol]-2′(1′H)-one.

Another embodiment is a compound of formula (I) which is a compound of formula (Ia), as set forth above, wherein:
j is 0 and k is 1;
Q is —O—;
A is C(R^{3a});
B is C(R^{3b});
E is C(R^{3e});
D is N;
R¹ is heterocyclylalkyl where the heterocyclylalkyl group is optionally substituted by one or more substituents selected from the group consisting of alkyl, halo, haloalkyl, —R⁸—OR⁵ and —R⁸—C(O)OR⁵;
R^{2a}, R^{2b}, R^{2c} and R^{2d} are each independently selected from the group consisting of hydrogen, alkyl, halo, haloalkyl and heteroaryl;
R^{3a}, R^{3b} and R^{3e} are each independently selected from the group consisting of hydrogen, alkyl, alkoxy, halo, haloalkyl and —R⁸—OR⁵;
or R^{3a} and R^{3b} or R^{3b} and R^{3e}, together with the carbon ring atoms to which they are directly attached, may form a fused ring selected from heterocyclyl;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

Another embodiment is a compound of formula (Ia) selected from the group consisting of:
5-methoxy-1′-(piperidin-4-ylmethyl)spiro[furo[3,2-b]pyridine-3,3′-indol]-2′(1′H)-one;
tert-butyl 4-[(4′-bromo-5-methoxy-2′-oxospiro[furo[3,2-b]pyridine-3,3′-indol]-1′(2′H)-yl)methyl]piperidine-1-carboxylate; and
5-methoxy-1-[(1-methylpiperidin-4-yl)methyl]spiro[furo[3,2-b]pyridine-3,3′-indol]-2′(1′H)-one.

Another embodiment is a compound of formula (I) which is a compound of formula (Ia), as set forth above, wherein:
j is 0 and k is 1;
Q is —O—;
A is C(R^{3a});
B is C(R^{3b});
E is C(R^{3e});
D is N;
R¹ is —R⁹—C(O)N(R¹²)R¹¹ where:
   R¹¹ is hydrogen, alkyl, aryl or aralkyl;
   R¹² is hydrogen, alkyl, aryl, aralkyl or —C(O)R⁵;
   and wherein each aryl or aralkyl groups for R¹¹ may be optionally substituted by one or more substituents selected from the group consisting of alkyl, cycloalkyl, aryl, aralkyl, halo and haloalkyl;
R^{2a}, R^{2b}, R^{2c} and R^{2d} are each independently selected from the group consisting of hydrogen, alkyl, halo, haloalkyl and heteroaryl;
R^{3a}, R^{3b} and R^{3e} are each independently selected from the group consisting of hydrogen, alkyl, alkoxy, halo, haloalkyl and —R⁸—OR⁵;
or R^{1a} and R^{3b} or R^{3b} and R^{3e}, together with the carbon ring atoms to which they are directly attached, may form a fused ring selected from heterocyclyl;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

Another embodiment is a compound of formula (Ia) which is N-(2-fluorophenyl)-2-(5-methoxy-2′-oxospiro[furo[3,2-b]pyridine-3,3′-indol]-1′(2′H)-yl)acetamide.

Another embodiment is a compound of formula (I) which is a compound of formula (Ia), as set forth above, wherein:
j and k are each independently 0, 1, 2 or 3;
Q is —C(R^{1a})H—, —C(O)—, —O—, —S—, —N(R⁵)—, —CF₂—, —C(O)O—, —C(O)N(R⁵)— or —N(R⁵)C(O)—;
A is C(R^{3a});
B is N;
E is C(R^{3e});
D is C(R^{3d});
R^{1a} is hydrogen or —OR⁵;
R¹ is hydrogen, alkyl, alkenyl, alkynyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
   R⁶ is hydrogen, alkyl, aryl or aralkyl; and
   R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
   or R⁶ and R⁷, together with the nitrogen to which they are attached, form a heterocyclyl or heteroaryl;
   and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is —R⁹—N(R¹⁰)R¹¹, —R⁹—N(R¹²)C(O)R¹¹ or —R⁹—N(R¹⁰)C(O)N(R¹⁰)R¹¹ where:
   each R¹⁰ is hydrogen, alkyl, aryl or aralkyl;
   each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—OR⁵ or —R⁹—CN;
   R¹² is hydrogen, alkyl, aryl, aralkyl or —C(O)R⁵;
   and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(═N—CN)N(R⁴)R⁵, wherein each m is independently 0, 1, or 2 and each n is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{2a} and R^{2b}, or R^{2b} and R^{2c}, or R^{2c} and R^{2d}, together with the carbon ring atoms to which they are directly attached, may form a fused ring selected from cycloalkyl, aryl, heterocyclyl and heteroaryl;
R^{3a}, R^{3e} and R^{3d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(N═C(R⁴)R⁵)N(R⁴)R⁵, wherein each m is independently 0, 1, or 2 and each n is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3e} and R^{3d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3e} and R^{3d}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

Another embodiment is a compound of formula (I) which is a compound of formula (Ia), as set forth above, wherein:
j is 0 and k is 1;
Q is —C(R^{1a})H—, —O—, —S— or —N(R⁵)—;
A is C(R^{3a});
B is N;
E is C(R^{3e});
D is C(R^{3d});
R^{1a} is hydrogen or —OR⁵;
R¹ is hydrogen, alkyl, alkenyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkoxy, halo, haloalkyl, haloalkenyl, haloalkoxy, cycloalkyl, cycloalkylalkyl, aryl, aralkyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴ and —R⁸—C(O)N(R⁴)R⁵;
   and wherein each of the cycloalkyl, cycloalkylalkyl, aryl, aralkyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein m is 0, 1, or 2 and n is 1 or 2;
R^{3a}, R^{3e} and R^{3d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴,
   and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3e} and R^{3d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3e} and R^{3d}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain; and
each R⁹ is a straight or branched alkylene chain.

Another embodiment is a compound of formula (I) which is a compound of formula (Ia), as set forth above, wherein:
j is 0 and k is 1;
Q is —O—;
A is C(R^{3a});
B is N;
E is C(R^{3e});
D is C(R^{3d});
R¹ is hydrogen, alkyl, alkenyl, haloalkyl, —R⁸—OR⁵ or —R⁸—CN;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkoxy, halo, haloalkyl, heteroaryl, —R⁸—OR⁵, —R⁸—N(R⁴)R⁵, —R⁸—C(O)R⁴, —R⁸—C(O)OR⁴ and —R⁸—C(O)N(R⁴)R⁵;
R^{3a}, R^{3e} and R^{3d} are each independently selected from the group consisting of hydrogen, alkyl, alkoxy, halo, haloalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴;
or R^{3e} and R^{3d}, together with the carbon ring atoms to which they are directly attached, may form a fused heterocyclyl ring;
each R⁴ and R⁵ is independently selected from group consisting of hydrogen, alkyl, haloalkyl, alkoxyalkyl, cycloalkyl, cycloalkylalkyl, aryl, aralkyl, heterocyclyl and heteroaryl; and
each R⁸ is a direct bond or a straight or branched alkylene chain.

Another embodiment is a compound of formula (I) which is a compound of formula (Ia), as set forth above, wherein:
j is 0 and k is 1;
Q is —O—;
A is C(R^{3a});
B is N;
E is C(R^{3e});
D is C(R^{3d});
R¹ is hydrogen;
R^{2a}, R^{2b}, R^{2c} and R^{2d} are each independently selected from the group consisting of hydrogen, alkyl, halo, haloalkyl and heteroaryl;
R^{3a}, R^{3e} and R^{3d} are each independently selected from the group consisting of hydrogen, alkyl, alkoxy, halo, haloalkyl and —R⁸—OR⁵;
or R^{3e} and R^{3d}, together with the carbon ring atoms to which they are directly attached, may form a fused ring selected from heterocyclyl;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

Another embodiment is a compound of formula (Ia) which is 5-methoxyspiro[furo[2,3-c]pyridine-3,3′-indol]-2′(1′H)-one.

Another embodiment is a compound of formula (I) which is a compound of formula (Ia), as set forth above, wherein:
j is 0 and k is 1;
Q is —O—;
A is C(R^{3a});
B is N;
E is C(R^{3e});
D is C(R^{3d});
R¹ is heteroarylalkyl where the heteroarylalkyl group is optionally substituted by one or more substituents selected from the group consisting of alkyl, halo, haloalkyl, —R⁸—OR⁵ and —R⁸—C(O)OR⁵;
R^{2a}, R^{2b}, R^{2c} and R^{2d} are each independently selected from the group consisting of hydrogen, alkyl, halo, haloalkyl and heteroaryl;
R^{3a}, R^{3e} and R^{3d} are each independently selected from the group consisting of hydrogen, alkyl, alkoxy, halo, haloalkyl and —R⁸—OR⁵;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

Another embodiment is a compound of formula (Ia) which is 5-methoxy-1′-{[5-(trifluoromethyl)furan-2-yl]methyl}spiro[furo[2,3-c]pyridine-3,3′-indol]-2′(1′H)-one;

Another embodiment is a compound of formula (I) which is a compound of formula (Ia), as set forth above, wherein:
j is 0 and k is 1;
Q is —O—;
A is C(R^{3e});
B is N;
E is C(R^{3e});
D is C(R^{3d});
R¹ is heterocyclylalkyl where the heterocyclylalkyl group is optionally substituted by one or more substituents selected from the group consisting of alkyl, halo, haloalkyl, —R⁸—OR⁵ and —R⁸—C(O)OR⁵;
R^{2a}, R^{2b}, R^{2c} and R^{2d} are each independently selected from the group consisting of hydrogen, alkyl, halo, haloalkyl and heteroaryl;
R^{3a}, R^{3e} and R^{3d} are each independently selected from the group consisting of hydrogen, alkyl, alkoxy, halo, haloalkyl and —R⁸—OR⁵;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

Another embodiment is a compound of formula (Ia) selected from the group consisting of:
5-methoxy-1′-(tetrahydro-2H-pyran-4-ylmethyl)spiro[furo[2,3-c]pyridine-3,3′-indol]-2′(1′H)-one;
5-methoxy-1′-[(2S)-tetrahydrofuran-2-ylmethyl]spiro[furo[2,3-c]pyridine-3,3′-indol]-2′(1′H)-one; and
5-methoxy-1′-[(2R)-tetrahydrofuran-2-ylmethyl]spiro[furo[2,3-c]pyridine-3,3′-indol]-2′(1′H)-one.

Another embodiment is a compound of formula (I) which is a compound of formula (Ia), as set forth above, wherein:
j is 0 and k is 1;
Q is —O—;
A is C(R^{3a});
B is N;
E is C(R^{3e});
D is C(R^{3d});
R¹ is aralkyl;
R^{2a}, R^{2b}, R^{2c} and R^{2d} are each independently selected from the group consisting of hydrogen, alkyl, halo, haloalkyl and heteroaryl;
R^{3a}, R^{3e} and R^{3d} are each independently selected from the group consisting of hydrogen, alkyl, alkoxy, halo, haloalkyl and —R⁸—OR⁵;
or R^{3e} and R^{3d}, together with the carbon ring atoms to which they are directly attached, may form a fused ring selected from heterocyclyl;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

Another embodiment is a compound of formula (Ia) which is (diphenylmethyl)-5-methoxyspiro[furo[2,3-c]pyridine-3,3′-indol]-2′(1′H)-one.

Another embodiment is a compound of formula (I) which is a compound of formula (Ia), as set forth above, wherein:
j and k are each independently 0, 1, 2 or 3;
Q is —C(R^{1a})H—, —C(O)—, —O—, —S—, —N(R⁵)—, —CF₂—, —C(O)O—, —C(O)N(R⁵)— or —N(R⁵)C(O)—;
A is C(R^{3a});
B is C(R^{3b});
E is N;
D is C(R^{3d});
R^{1a} is hydrogen or —OR⁵;
R¹ is hydrogen, alkyl, alkenyl, alkynyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
   R⁶ is hydrogen, alkyl, aryl or aralkyl; and
   R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
   or R⁶ and R⁷, together with the nitrogen to which they are attached, form a heterocyclyl or heteroaryl;
   and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is —R⁹—N(R¹⁰)R¹¹, —R⁹—N(R¹²)C(O)R¹¹ or —R⁹—N(R¹⁰)C(O)N(R¹⁰)R¹¹ where:
   each R¹⁰ is hydrogen, alkyl, aryl or aralkyl;
   each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—OR⁵ or —R⁹—CN;
   R¹² is hydrogen, alkyl, aryl, arylalkyl or —C(O)R⁵;
   and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(═N—CN)N(R⁴)R⁵, wherein each m is independently 0, 1, or 2 and each n is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{2a} and R^{2b}, or R^{2b} and R^{2c}, or R^{2c} and R^{2d}, together with the carbon ring atoms to which they are directly attached, may form a fused ring selected from cycloalkyl, aryl, heterocyclyl and heteroaryl;
R^{3a}, R^{3b} and R^{3d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(N═C(R⁴)R⁵)N(R⁴)R⁵, wherein each m is independently 0, 1, or 2 and each n is independently 1 or 2,
   and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3b} and R^{3d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3a} and R^{3b}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

Another embodiment is a compound of formula (I) which is a compound of formula (Ia), as set forth above, wherein:
j is 0 and k is 1;
Q is —O—;
A is C(R^{3a});
B is C(R^{3b});
E is N;
D is C(R^{3d});
R¹ is hydrogen, alkyl, alkenyl, alkynyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
R^{2a}, R^{2b}, R^{2c} and R^{2d} are each independently selected from the group consisting of hydrogen, alkyl, halo, haloalkyl and heteroaryl;
R^{3a}, R^{3b} and R^{3d} are each independently selected from the group consisting of hydrogen, alkyl, alkoxy, halo, haloalkyl and —R⁸—OR⁵;
or R^{3a} and R^{3b}, together with the carbon ring atoms to which they are directly attached, may form a fused ring selected from heterocyclyl;
each R⁴ and R⁵ is independently selected from group consisting of hydrogen, alkyl, haloalkyl, alkoxyalkyl, aryl and aralkyl;
each R⁸ is a direct bond or a straight or branched alkylene chain; and
each R⁹ is a straight or branched alkylene chain, a straight or branched alkenylene chain or a straight or branched alkynylene chain.

Another embodiment is a compound of formula (I) which is a compound of formula (Ia), as set forth above, wherein:
j is 0 and k is 1;
Q is —O—;
A is C(R^{3a});
B is C(R^{3b});
E is N;
D is C(R^{3d});
R¹ is hydrogen or alkyl;
R^{2a}, R^{2b}, R^{2c} and R^{2d} are each independently selected from the group consisting of hydrogen, alkyl, halo, haloalkyl and heteroaryl;
R^{3a}, R^{3b} and R^{3d} are each independently selected from the group consisting of hydrogen, alkyl, alkoxy, halo, haloalkyl and —R⁸—OR⁵;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

Another embodiment is a compound of formula (Ia) which is 1′-pentylspiro[furo[3,2-c]pyridine-3,3′-indol]-2′(1′H)-one.

Another embodiment is a compound of formula (I) which is a compound of formula (Ia), as set forth above, wherein:
j and k are each independently 0, 1, 2 or 3;
Q is —C(R^{1a})H—, —C(O)—, —O—, —S—, —N(R⁵)—, —CF₂—, —C(O)O—, —C(O)N(R⁵)— or —N(R⁵)C(O)—;
A is C(R^{3a});
B is C(R^{3b});
D is C(R^{3d});
E is N→O;
R^{1a} is hydrogen or —OR⁵;
R¹ is hydrogen, alkyl, alkenyl, alkynyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
   R⁶ is hydrogen, alkyl, aryl or aralkyl; and
   R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
   or R⁶ and R⁷, together with the nitrogen to which they are attached, form a heterocyclyl or heteroaryl;
   and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
   each R¹⁰ is hydrogen, alkyl, aryl or aralkyl;
   each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—OR⁵ or —R⁹—CN;
   R¹² is hydrogen, alkyl, aryl, aralkyl or —C(O)R⁵;
   and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(═N—CN)N(R⁴)R⁵, wherein each m is independently 0, 1, or 2 and each n is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{2a} and R^{2b}, or R^{2b} and R^{2c}, or R^{2c} and R^{2d}, together with the carbon ring atoms to which they are directly attached, may form a fused ring selected from cycloalkyl, aryl, heterocyclyl and heteroaryl;
R^{3a}, R^{3b} and R^{3d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(N═C(R⁴)R⁵)N(R⁴)R⁵, wherein each m is independently 0, 1, or 2 and each n is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3b} and R^{3d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3a} and R^{3b}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

Another embodiment is a compound of formula (I) which is a compound of formula (Ia), as set forth above, wherein:
j is 0 and k is 1;
Q is —C(R^{1a})H—, —O—, —S— or —N(R⁵)—;
A is C(R^{3a});
B is C(R^{3b});
D is C(R^{3d});
E is N→O;
R^{1a} is hydrogen or —OR⁵;
R¹ is hydrogen, alkyl, alkenyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
R^{2a}, R^{2b}, R^{2c} and R^{2d} are each independently selected from the group consisting of hydrogen, alkyl, alkenyl, alkoxy, halo, haloalkyl, haloalkenyl, haloalkoxy, cycloalkyl, cycloalkylalkyl, aryl, aralkyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)S(O)ₙR⁴ wherein n is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
R^{3a}, R^{3b} and R^{3d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴,
   and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3b} and R^{3d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

Another embodiment is a compound of formula (I) which is a compound of formula (Ia), as set forth above, wherein:
j is 0 and k is 1;
Q is —O—;
A is C(R^{3a});
B is C(R^{3b});
D is C(R^{3d});
E is N→O;
R¹ is hydrogen, alkyl, alkenyl or haloalkyl;
R^{2a}, R^{2b}, R^{2c} and R^{2d} are each independently selected from the group consisting of hydrogen, alkyl, alkenyl, alkoxy, halo, haloalkyl, —R⁸—OR⁵, —R⁸—N(R⁴)R⁵, —R⁸—C(O)R⁴, —R⁸—C(O)OR⁴ and —R⁸—C(O)N(R⁴)R⁵;
R^{3a}, R^{3b} and R^{3d} are each independently selected from the group consisting of hydrogen, alkyl, alkenyl, alkoxy, halo, haloalkyl, haloalkoxy, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain.

Another embodiment is a compound of formula (Ia) which is 1′-pentylspiro[furo[3,2-c]pyridine-3,3′-indol]-2′(1′H)-one 5-oxide.

Another embodiment is a compound of formula (I) which is a compound of formula (Ia), as set forth above, wherein:
j and k are each independently 0, 1, 2 or 3;
Q is —C(R^{1a})H—, —C(O)—, —O—, —S—, —N(R⁵)—, —CF₂—, —C(O)O—, —C(O)N(R⁵)— or —N(R⁵)C(O)—;
A is C(R^{3a});
B is C(R^{3b});
D is N→O;
E is C(R^{3e});
R^{1a} is hydrogen or —OR⁵;
R¹ is hydrogen, alkyl, alkenyl, alkynyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
   R⁶ is hydrogen, alkyl, aryl or aralkyl; and
   R⁷ is hydrogen, alkyl, haloalkyl, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
   or R⁶ and R⁷, together with the nitrogen to which they are attached, form a heterocyclyl or heteroaryl;
   and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, heterocyclyl and heteroaryl;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
   each R¹⁰ is hydrogen, alkyl, aryl or aralkyl;
   each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—OR⁵ or —R⁹—CN;
   R¹² is hydrogen, alkyl, aryl, aralkyl or —C(O)R⁵;
   and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—C(O)OR⁵, aryl and aralkyl;
R^{2a}, R^{2c} and R^{2d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(═N—CN)N(R⁴)R⁵, wherein each m is independently 0, 1, or 2 and each n is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{2a} and R^{2b}, or R^{2b} and R^{2c}, or R^{2c} and R^{2d}, together with the carbon ring atoms to which they are directly attached, may form a fused ring selected from cycloalkyl, aryl, heterocyclyl and heteroaryl;
R^{3a}, R^{3b} and R^{3e} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵, and —N(R⁵)C(N═C(R⁴)R⁵)N(R⁴)R⁵, wherein each m is independently 0, 1, or 2 and each n is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3b} and R^{3e} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴, and —N(R⁵)S(O)ₙR⁴, wherein each m is independently 0, 1, or 2 and each n is independently 1 or 2;
or R^{3a} and R^{3b}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

Another embodiment is a compound of formula (I) which is a compound of formula (Ia), as set forth above, wherein:
j is 0 and k is 1;
Q is —C(R^{1a})H—, —O—, —S— or —N(R⁵)—;
A is C(R^{3a});
B is C(R^{3b});
D is N→O;
E is C(R^{3e});
R^{1a} is hydrogen or —OR⁵;
R¹ is hydrogen, alkyl, alkenyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkoxy, halo, haloalkyl, haloalkenyl, haloalkoxy, cycloalkyl, cycloalkylalkyl, aryl, aralkyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴ and —R⁸—C(O)N(R⁴)R⁵ and —N(R⁵)S(O)ₙR⁴ wherein n is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
R^{3a}, R^{3b} and R^{3e} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴,
   and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3b} and R^{3e} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

Another embodiment is a compound of formula (I) which is a compound of formula (Ia), as set forth above, wherein:
j is 0 and k is 1;
Q is —O—;
A is C(R^{3a});
B is C(R^{3b});
D is N→O;
E is C(R^{3e});
R¹ is hydrogen, alkyl, alkenyl, or haloalkyl;
or R¹ is heteroarylalkyl where the heterocyclylalkyl or the heteroarylalkyl group is optionally substituted by one or more substituents selected from the group consisting of alkyl, halo, haloalkyl, —R⁸—OR⁵, and —R⁸—C(O)OR⁵;
R^{2a}, R^{2b}, R^{2c} and R^{2d} are each independently selected from the group consisting of hydrogen, alkyl, alkenyl, alkoxy, halo, haloalkyl, —R⁸—OR⁵, —R⁸—N(R⁴)R⁵, —R⁸—C(O)R⁴, —R⁸—C(O)OR⁴ and —R⁸—C(O)N(R⁴)R⁵;
R^{3a}, R^{3b} and R^{3e} are each independently selected from the group consisting of hydrogen, alkyl, alkenyl, alkoxy, halo, haloalkyl, haloalkoxy, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain.

Another embodiment is a compound of formula (Ia) which is 5-methoxy-1′-{[5-(trifluoromethyl)-2-furyl]methyl}spiro[furo[3,2-b]pyridine-3,3′-indol]-2′(1′H)-one 4-oxide.

Another embodiment is a compound of formula (I) which is a compound of formula (Ia), as set forth above, wherein:
j and k are each independently 0, 1, 2 or 3;
Q is —C(R^{1a})H—, —C(O)—, —O—, —S—, —N(R⁵)—, —CF₂—, —C(O)O—, —C(O)N(R⁵)— or —N(R⁵)C(O)—;
A is C(R^{3a}), B is C(R^{3b}), E is N(H) and D is C(O);
or A is C(R^{3a}), B is C(R^{3b}), D is N(H) and E is C(O);
R^{1a} is hydrogen or —OR⁵;
R¹ is hydrogen, alkyl, alkenyl, alkynyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
   R⁶ is hydrogen, alkyl, aryl or aralkyl; and
   R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
   or R⁶ and R⁷, together with the nitrogen to which they are attached, form a heterocyclyl or heteroaryl;
   and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
   each R¹⁰ is hydrogen, alkyl, aryl or aralkyl;
   each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—OR⁵ or —R⁹—CN;
R¹² is hydrogen, alkyl, aryl, aralkyl or —C(O)R⁵;
   and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(═N—CN)N(R⁴)R⁵, wherein each m is independently 0, 1, or 2 and each n is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{2a} and R^{2b}, or R^{2b} and R^{2c}, or R^{2c} and R^{2d}, together with the carbon ring atoms to which they are directly attached, may form a fused ring selected from cycloalkyl, aryl, heterocyclyl and heteroaryl;
R^{3a} and R^{3b} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(N═C(R⁴)R⁵)N(R⁴)R⁵, wherein each m is independently 0, 1, or 2 and each n is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{3a} and R^{3b} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3a} and R^{3b}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

Another embodiment is a compound of formula (I) which is a compound of formula (Ia), as set forth above, wherein:
j is 0 and k is 1;
Q is —C(R^{1a})H—, —O—, —S— or —N(R⁵)—;
A is C(R^{3a}), B is C(R^{3b}), E is N(H) and D is C(O);
or A is C(R^{3a}), B is C(R^{3b}), D is N(H) and E is C(O);
R^{1a} is hydrogen or —OR⁵;
R¹ is hydrogen, alkyl, alkenyl, haloalkyl, aryl, cycloalkyl, cycloalkylalkyl, heteroaryl, heterocyclyl, —R⁸—OR⁵, —R⁸—CN, —R⁹—P(O)(OR⁵)₂, or —R⁹—O—R⁹—OR⁵;
R^{2a}, R^{2b}, R^{2c} and R^{2d} are each independently selected from the group consisting of hydrogen, alkyl, alkenyl, alkoxy, halo, haloalkyl, haloalkenyl, haloalkoxy, cycloalkyl, cycloalkylalkyl, aryl, aralkyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)S(O)ₙR⁴ wherein n is independently 1 or 2;
   and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
R^{3a} and R^{3b} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴,
   and wherein each of the cycloalkyl, cycloalkylalkyl, aryl, aralkyl, aralkenyl, heterocyclyl, heterocyclylalkyl, heteroaryl and heteroarylalkyl groups for R^{3a} and R^{3b} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

Another embodiment is a compound of formula (I) which is a compound of formula (Ia), as set forth above, wherein:
j is 0 and k is 1;
Q is —O—;
A is C(R^{3a}), B is C(R^{3b}), E is N(H) and D is C(O);
or A is C(R^{3a}), B is C(R^{3b}), D is N(H) and E is C(O);
R¹ is hydrogen, alkyl, alkenyl, or haloalkyl;
R^{2a}, R^{2b}, R^{2c} and R^{2d} are each independently selected from the group consisting of hydrogen, alkyl, alkenyl, alkoxy, halo, haloalkyl, —R⁸—OR⁵, —R⁸—N(R⁴)R⁵, —R⁸—C(O)R⁴, —R⁸—C(O)OR⁴ and —R⁸—C(O)N(R⁴)R⁵;
R^{3a}, R^{3b} and R^{3e} are each independently selected from the group consisting of hydrogen, alkyl, alkenyl, alkoxy, halo, haloalkyl, haloalkoxy, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain.

Another embodiment is a compound of formula (I) which is a compound of formula (Ia), as set forth above, wherein:
j is 0 and k is 1;
Q is —O—;
A is C(R^{3a}), B is C(R^{3b}), E is N(H) and D is C(O);
R¹ is hydrogen, alkyl, alkenyl, or haloalkyl;
R^{2a}, R^{2b}, R^{2c} and R^{2d} are each independently selected from the group consisting of hydrogen, alkyl, alkenyl, alkoxy, halo, haloalkyl, —R⁸—OR⁵, —R⁸—N(R⁴)R⁵, —R⁸—C(O)R⁴, —R⁸—C(O)OR⁴ and —R⁸—C(O)N(R⁴)R⁵;
R^{3a}, R^{3b} and R^{3e} are each independently selected from the group consisting of hydrogen, alkyl, alkenyl, alkoxy, halo, haloalkyl, haloalkoxy, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain.

Another embodiment is a compound of formula (Ia) which is pentylspiro[furo[3,2-c]pyridine-3,3′-indole]-2′,4(1′H,5H)-dione.

Another embodiment is a compound of formula (I) which is a compound of formula (Ia), as set forth above, wherein:
j is 0 and k is 1;
Q is —O—;
A is C(R^{3a}), B is C(R^{3b}), D is N(H) and E is C(O);
R¹ is hydrogen, alkyl, alkenyl or haloalkyl;
R^{2a}, R^{2b}, R^{2c} and R^{2d} are each independently selected from the group consisting of hydrogen, alkyl, alkenyl, alkoxy, halo, haloalkyl, —R⁸—OR⁵, —R⁸—N(R⁴)R⁵, —R⁸—C(O)R⁴, —R⁸—C(O)OR⁴ and —R⁸—C(O)N(R⁴)R⁵;
R^{3a}, R^{3b} and R^{3e} are each independently selected from the group consisting of hydrogen, alkyl, alkenyl, alkoxy, halo, haloalkyl, haloalkoxy, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain.

Another embodiment is a compound of formula (Ia) which is 1′-{[5-(trifluoromethyl)-2-furyl]methyl}spiro[furo[3,2-b]pyridine-3,3′-indole]-2′,5(1′H,4H)-dione.

Embodiments of k, j, m, R¹, R^{2a}, R^{2b}, R^{2c}, R^{2d} and X are the same for the compounds of formula (I) wherein

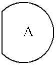

is a heterocyclyl as k, j, m, R¹, R^{2a}, R^{2b}, R^{2c}, R^{2d} and X embodiments set forth above for compounds of formula (Ia).

It is understood that any embodiment of the compounds of formula (Ia) as set forth above, and j, k, m or any specific substituent set forth herein for a R¹, R^{2a}, R^{2b}, R^{2c}, R^{2d}, R^{3a}, R^{3b}, R^{3c}, R^{3d}, A, B, D, and E group in the compounds of formula (Ia), as set forth above, may be independently combined with other embodiments and/or any j, k, m or any specific substituent set forth herein for a R¹, R^{2a}, R^{2b}, R^{2c}, R^{2d}, R^{3a}, R^{3b}, R^{3c}, R^{3d}, A, B, D, and E group in compounds of formula (Ia) to form embodiments of the inventions not specifically set forth above. In addition, in the event that a list of substitutents is listed for any particular R group or an A, B, D or E group in a particular embodiment and/or claim, it is understood that each individual substituent may be deleted from the particular embodiment and/or claim and that the remaining list of substituents will be considered to be enabled by the disclosure herein.

Another embodiment of the invention is a method of treating, preventing or ameliorating a disease or a condition in a mammal, preferably a human, wherein the disease or condition is selected from the group consisting of pain, depression, cardiovascular diseases, respiratory diseases, and psychiatric diseases, and combinations thereof, and wherein the method comprises administering to the mammal in need thereof a therapeutically effective amount of an embodiment of a compound of the invention, as set forth above, as a stereoisomer, enantiomer, tautomer thereof or mixtures thereof, or a pharmaceutically acceptable salt, N-oxide, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, and a pharmaceutically acceptable excipient.

One embodiment of this embodiment is wherein the disease or condition is selected from the group consisting of neuropathic pain, inflammatory pain, visceral pain, cancer pain, chemotherapy pain, trauma pain, surgical pain, post-surgical pain, childbirth pain, labor pain, neurogenic bladder, ulcerative colitis, chronic pain, persistent pain, peripherally mediated pain, centrally mediated pain, chronic headache, migraine headache, sinus headache, tension headache, phantom limb pain, peripheral nerve injury, and combinations thereof.

Another embodiment of this embodiment is wherein the disease or condition is selected from the group consisting of pain associated with HIV, HIV treatment induced neuropathy, trigeminal neuralgia, post-herpetic neuralgia, eudynia, heat sensitivity, tosarcoidosis, irritable bowel syndrome, Crohns disease, pain associated with multiple sclerosis (MS), amyotrophic lateral sclerosis (ALS), diabetic neuropathy, peripheral neuropathy, arthritic, rheumatoid arthritis, osteoarthritis, atherosclerosis, paroxysmal dystonia, myasthenia syndromes, myotonia, malignant hyperthermia, cystic fibrosis, pseudoaldosteronism, rhabdomyolysis, hypothyroidism, bipolar depression, anxiety, schizophrenia, sodium channel toxin related illnesses, familial erythermalgia, primary erythermalgia, familial rectal pain, cancer, epilepsy, partial and general tonic seizures, restless leg syndrome, arrhythmias, fibromyalgia, neuroprotection under ischaemic conditions caused by stroke or neural trauma, tachy-arrhythmias, atrial fibrillation and ventricular fibrillation.

Another embodiment of the invention is the method of treating pain in a mammal, preferably a human, by the inhibition of ion flux through a voltage-dependent sodium channel in the mammal, wherein the method comprises administering to the mammal in need thereof a therapeutically effective amount of an embodiment of a compound of the invention, as set forth above, as a stereoisomer, enantiomer, tautomer thereof or mixtures thereof, or a pharmaceutically acceptable salt, N-oxide, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, and a pharmaceutically acceptable excipient.

Another embodiment of the invention is the method of treating or preventing hypercholesterolemia in a mammal, preferably a human, wherein the method comprises administering to the mammal in need thereof a therapeutically effective amount of an embodiment of a compound of the invention, as set forth above, as a stereoisomer, enantiomer, tautomer thereof or mixtures thereof, or a pharmaceutically acceptable salt, N-oxide, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, and a pharmaceutically acceptable excipient.

Another embodiment of the invention is the method of treating or preventing benign prostatic hyperplasia in a mammal, preferably a human, wherein the method comprises administering to the mammal in need thereof a therapeutically effective amount of an embodiment of a compound of the invention, as set forth above, as a stereoisomer, enantiomer, tautomer thereof or mixtures thereof, or a pharmaceutically acceptable salt, N-oxide, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, and a pharmaceutically acceptable excipient.

Another embodiment of the invention is the method of treating or preventing pruritis in a mammal, preferably a human, wherein the method comprises administering to the mammal in need thereof a therapeutically effective amount of an embodiment of a compound of the invention, as set forth above, as a stereoisomer, enantiomer, tautomer thereof or mixtures thereof, or a pharmaceutically acceptable salt, N-oxide, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, and a pharmaceutically acceptable excipient.

Another embodiment of the invention is the method of treating or preventing cancer in a mammal, preferably a human, wherein the method comprises administering to the mammal in need thereof a therapeutically effective amount of an embodiment of a compound of the invention, as set forth above, as a stereoisomer, enantiomer, tautomer thereof or mixtures thereof, or a pharmaceutically acceptable salt, N-oxide, solvate or prodrug thereof, or a pharmaceutical composition comprising a therapeutically effective amount of a compound of the invention, as set forth above, as a stereoisomer, enantiomer, tautomer thereof or mixtures thereof, or a pharmaceutically acceptable salt, N-oxide, solvate or prodrug thereof, and a pharmaceutically acceptable excipient.

Another embodiment of the invention is the method of decreasing ion flux through a voltage-dependent sodium channel in a cell in a mammal, wherein the method comprises contacting the cell with an embodiment of a compound of the invention, as set forth above, as a stereoisomer, enantiomer, tautomer thereof or mixtures thereof, or a pharmaceutically acceptable salt, N-oxide, solvate or prodrug thereof.

Specific embodiments of the compounds of the invention are described in more detail below in the Preparation of the Compounds of Formula (I).

### Utility and Testing of the Compounds of the Invention

The compounds of the invention modulate, preferably inhibit, ion flux through a voltage-dependent sodium channel in a mammal, especially in a human. Any such modulation, whether it be partial or complete inhibition or prevention of ion flux, is sometimes referred to herein as “blocking” and corresponding compounds as “blockers”. In general, the compounds of the invention modulates the activity of a sodium channel downwards, inhibits the voltage-dependent activity of the sodium channel, and/or reduces or prevents sodium ion flux across a cell membrane by preventing sodium channel activity such as ion flux.

The compounds of the invention inhibit the ion flux through a voltage-dependent sodium channel. Preferably, the compounds are state or frequency dependent modifiers of the sodium channels, having a low affinity for the rested/closed state and a high affinity for the inactivated state. These compounds are likely to interact with overlapping sites located in the inner cavity of the sodium conducting pore of the channel similar to that described for other state-dependent sodium channel blockers (Cestele, S., et al., op. cit.). These compounds may also be likely to interact with sites outside of the inner cavity and have allosteric effects on sodium ion conduction through the channel pore.

Any of these consequences may ultimately be responsible for the overall therapeutic benefit provided by these compounds.

Accordingly, the compounds of the invention are sodium channel blockers and are therefore useful for treating diseases and conditions in mammals, preferably humans, and other organisms, including all those human diseases and conditions which are the result of aberrant voltage-dependent sodium channel biological activity or which may be ameliorated by modulation of voltage-dependent sodium channel biological activity.

As defined herein, a sodium channel-mediated disease or condition refers to a disease or condition in a mammal, preferably a human, which is ameliorated upon modulation of the sodium channel and includes, but is not limited to, pain, central nervous conditions such as epilepsy, anxiety, depression and bipolar disease; cardiovascular conditions such as arrhythmias, atrial fibrillation and ventricular fibrillation; neuromuscular conditions such as restless leg syndrome and muscle paralysis or tetanus; neuroprotection against stroke, neural trauma and multiple sclerosis; and channelopathies such as erythromyalgia and familial rectal pain syndrome.

The present invention therefore relates to compounds, pharmaceutical compositions and methods of using the compounds and pharmaceutical compositions for the treatment of sodium channel-mediated diseases in mammals, preferably humans and preferably diseases related to pain, central nervous conditions such as epilepsy, anxiety, depression and bipolar disease; cardiovascular conditions such as arrhythmias, atrial fibrillation and ventricular fibrillation; neuromuscular conditions such as restless leg syndrome and muscle paralysis or tetanus; neuroprotection against stroke, neural trauma and multiple sclerosis; and channelopathies such as erythromyalgia and familial rectal pain syndrome, by administering to a mammal, preferably a human, in need of such treatment an effective amount of a sodium channel blocker modulating, especially inhibiting, agent.

Accordingly, the present invention provides a method for treating a mammal for, or protecting a mammal from developing, a sodium channel-mediated disease, especially pain, comprising administering to the mammal, especially a human, in need thereof, a therapeutically effective amount of a compound of the invention or a pharmaceutical composition comprising a therapeutically effective amount of a compound of the invention wherein the compound modulates the activity of one or more voltage-dependent sodium channels.

The general value of the compounds of the invention in mediating, especially inhibiting, the sodium channel ion flux can be determined using the assays described below in the Biological Assays section. Alternatively, the general value of the compounds in treating conditions and diseases in humans may be established in industry standard animal models for demonstrating the efficacy of compounds in treating pain. Animal models of human neuropathic pain conditions have been developed that result in reproducible sensory deficits (allodynia, hyperalgesia, and spontaneous pain) over a sustained period of time that can be evaluated by sensory testing. By establishing the degree of mechanical, chemical, and temperature induced allodynia and hyperalgesia present, several physiopathological conditions observed in humans can be modeled allowing the evaluation of pharmacotherapies.

In rat models of peripheral nerve injury, ectopic activity in the injured nerve corresponds to the behavioural signs of pain. In these models, intravenous application of the sodium channel blocker and local anesthetic lidocaine can suppress the ectopic activity and reverse the tactile allodynia at concentrations that do not affect general behaviour and motor function (Mao, J. and Chen, L. L, Pain (2000), 87:7-17). Allimetric scaling of the doses effective in these rat models, translates into doses similar to those shown to be efficacious in humans (Tanelian, D. L. and Brose, W. G., Anesthesiology (1991), 74(5):949-951). Furthermore, Lidoderm®, lidocaine applied in the form of a dermal patch, is currently an FDA approved treatment for post-herpetic neuralgia (Devers, A. and Glaler, B. S., Clin. J. Pain (2000), 16(3):205-8).

A sodium channel-mediated disease or condition also includes pain associated with HIV, HIV treatment induced neuropathy, trigeminal neuralgia, glossopharyngeal neuralgia, neuropathy secondary to metastatic infiltration, adiposis dolorosa, thalamic lesions, hypertension, autoimmune disease, asthma, drug addiction (e.g. opiate, benzodiazepine, amphetamine, cocaine, alcohol, butane inhalation), Alzheimer, dementia, age-related memory impairment, Korsakoff syndrome, restenosis, urinary dysfunction, incontinence, Parkinson's disease, cerebrovascular ischemia, neurosis, gastrointestinal disease, sickle cell anemia, transplant rejection, heart failure, myocardial infarction, reperfusion injury, intermittant claudication, angina, convulsion, respiratory disorders, cerebral or myocardial ischemias, long-QT syndrome, Catecholeminergic polymorphic ventricular tachycardia, ophthalmic diseases, spasticity, spastic paraplegia, myopathies, myasthenia gravis, paramyotonia congentia, hyperkalemic periodic paralysis, hypokalemic periodic paralysis, alopecia, anxiety disorders, psychotic disorders, mania, paranoia, seasonal affective disorder, panic disorder, obsessive compulsive disorder (OCD), phobias, autism, Aspergers Syndrome, Retts syndrome, disintegrative disorder, attention deficit disorder, aggressivity, impulse control disorders, thrombosis, pre clampsia, congestive cardiac failure, cardiac arrest, Freidrich's ataxia, Spinocerebellear ataxia, myelopathy, radiculopathy, systemic lupus erythamatosis, granulomatous disease, olivo-ponto-cerebellar atrophy, spinocerebellar ataxia, episodic ataxia, myokymia, progressive pallidal atrophy, progressive supranuclear palsy and spasticity, traumatic brain injury, cerebral oedema, hydrocephalus injury, spinal cord injury, anorexia nervosa, bulimia, Prader-Willi syndrome, obesity, optic neuritis, cataract, retinal haemorrhage, ischaemic retinopathy, retinitis pigmentosa, acute and chronic glaucoma, macular degeneration, retinal artery occlusion, Chorea, Huntington's chorea, cerebral edema, proctitis, post-herpetic neuralgia, eudynia, heat sensitivity, sarcoidosis, irritable bowel syndrome, Tourette syndrome, Lesch-Nyhan Syndrome, Brugado syndrome, Liddle syndrome, Crohns disease, multiple sclerosis and the pain associated with multiple sclerosis (MS), amyotrophic lateral sclerosis (ALS), disseminated sclerosis, diabetic neuropathy, peripheral neuropathy, charcot marie tooth syndrome, arthritic, rheumatoid arthritis, osteoarthritis, chondrocalcinosis, atherosclerosis, paroxysmal dystonia, myasthenia syndromes, myotonia, myotonic dystrophy, muscular dystrophy, malignant hyperthermia, cystic fibrosis, pseudoaldosteronism, rhabdomyolysis, mental handicap, hypothyroidism, bipolar depression, anxiety, schizophrenia, sodium channel toxin related illnesses, familial erythermalgia, primary erythermalgia, rectal pain, cancer, epilepsy, partial and general tonic seizures, febrile seizures, absence seizures (petit mal), myoclonic seizures, atonic seizures, clonic seizures, Lennox Gastaut, West Syndome (infantile spasms), multiresistant seizures, seizure prophylaxis (anti-epileptogenic), familial Mediterranean fever syndrome, gout, restless leg syndrome, arrhythmias, fibromyalgia, neuroprotection under ischaemic conditions caused by stroke or neural trauma, tachy-arrhythmias, atrial fibrillation and ventricular fibrillation and as a general or local anaesthetic.

As used herein, the term “pain” refers to all categories of pain and is recognized to include, but is not limited to, neuropathic pain, inflammatory pain, nociceptive pain, idiopathic pain, neuralgic pain, orofacial pain, burn pain, burning mouth syndrome, somatic pain, visceral pain, myofacial pain, dental pain, cancer pain, chemotherapy pain, trauma pain, surgical pain, post-surgical pain, childbirth pain, labor pain, reflex sympathetic dystrophy, brachial plexus avulsion, neurogenic bladder, acute pain (e.g. musculoskeletal and post-operative pain), chronic pain, persistent pain, peripherally mediated pain, centrally mediated pain, chronic headache, migraine headache, familial hemiplegic migraine, conditions associated with cephalic pain, sinus headache, tension headache, phantom limb pain, peripheral nerve injury, pain following stroke, thalamic lesions, radiculopathy, HIV pain, post-herpetic pain, non-cardiac chest pain, irritable bowel syndrome and pain associated with bowel disorders and dyspepsia, and combinations thereof.

Sodium channel blockers have clinical uses in addition to pain. Epilepsy and cardiac arrhythmias are often targets of sodium channel blockers. Recent evidence from animal models suggest that sodium channel blockers may also be useful for neuroprotection under ischaemic conditions caused by stroke or neural trauma and in patients with multiple sclerosis (MS) (Clare, J. J. et al., op. cit. and Anger, T. et al., op. cit.).

The present invention also relates to compounds, pharmaceutical compositions and methods of using the compounds and pharmaceutical compositions for the treatment or prevention of diseases or conditions such as benign prostatic hyperplasia (BPH), hypercholesterolemia, cancer and pruritis (itch).

Benign prostatic hyperplasia (BPH), also known as benign prostatic hypertrophy, is one of the most common diseases affecting aging men. BPH is a progressive condition which is characterized by a nodular enlargement of prostatic tissue resulting in obstruction of the urethra. Consequences of BPH can include hypertrophy of bladder smooth muscle, a decompensated bladder, acute urinary retention and an increased incidence of urinary tract infection.

BPH has a high public health impact and is one of the most common reasons for surgical intervention among elderly men. Attempts have been made to clarify the etiology and pathogenesis and, to that end, experimental models have been developed. Spontaneous animal models are limited to the chimpanzee and the dog. BPH in man and the dog share many common features. In both species, the development of BPH occurs spontaneously with advanced age and can be prevented by early/prepubertal castration. A medical alternative to surgery is very desirable for treating BHP and the consequences.

The prostatic epithelial hyperplasia in both man and the dog is androgen sensitive, undergoing involution with androgen deprivation and resuming epithelial hyperplasia when androgen is replaced. Cells originating from the prostate gland have been shown to express high levels of voltage gated sodium channels. Immunostaining studies clearly demonstrated evidence for voltage gated sodium channels in prostatic tissues (Prostate Cancer Prostatic Dis. 2005; 8(3):266-73).

Hypercholesterolemia, i.e., elevated blood cholesterol, is an established risk factor in the development of, e.g., atherosclerosis, coronary artery disease, hyperlipidemia, stroke, hyperinsulinemias, hypertension, obesity, diabetes, cardiovascular diseases (CVD), myocardial ischemia, and heart attack. Thus, lowering the levels of total serum cholesterol in individuals with high levels of cholesterol has been known to reduce the risk of these diseases. The lowering of low density lipoprotein cholesterol in particular is an essential step in the prevention of CVD. Although there are a variety of hypercholesterolemia therapies, there is a continuing need and a continuing search in this field of art for alternative therapies.

The invention provides compounds which are useful as antihypercholesterolemia agents and their related conditions. The present compounds may act in a variety of ways. While not wishing to be bound to any particular mechanism of action, the compounds may be direct or indirect inhibitors of the enzyme acyl CoA: cholesterol acyl transferase (ACAT) that results in inhibition of the esterification and transport of cholesterol across the intestinal wall. Another possibility may be that the compounds of the invention may be direct or indirect inhibitors of cholesterol biosynthesis in the liver. It is possible that some compounds of the invention may act as both direct or indirect inhibitors of ACAT and cholesterol biosynthesis.

Pruritus, commonly known as itch, is a common dermatological condition. While the exact causes of pruritis are complex and poorly understood, there has long been acknowledged to have interactions with pain. In particular, it is believed that sodium channels likely communicate or propagate along the nerve axon the itch signals along the skin. Transmission of the itch impulses results in the unpleasant sensation that elicits the desire or reflex to scratch.

From a neurobiology level, it is believed that there is a shared complexity of specific mediators, related neuronal pathways and the central processes of itch and pain and recent data suggest that there is a broad overlap between pain- and itch-related peripheral mediators and/or receptors (Ikoma et al., Nature Reviews Neuroscience, 7:535-547, 2006). Remarkably, pain and itch have similar mechanisms of neuronal sensitization in the peripheral nervous system and the central nervous system but exhibits intriguing differences as well.

For example, the mildly painful stimuli from scratching are effective in abolishing the itch sensation. In contrast, analgesics such as opioids can generate severe pruritus. The antagonistic interaction between pain and itch can be exploited in pruritus therapy, and current research concentrates on the identification of common targets for future analgesic and antipruritic therapy.

Compounds of the present invention have been shown to have analgesic effects in a number of animal models at oral doses ranging from 1 mg/kg to 100 mg/kg. The compounds of the invention can also be useful for treating pruritus.

The types of itch or skin irritation, include, but are not limited to:

a) psoriatic pruritis, itch due to hemodyalisis, aguagenic pruritus, and itching caused by skin disorders (e.g., contact dermatitis), systemic disorders, neuropathy, psychogenic factors or a mixture thereof;

b) itch caused by allergic reactions, insect bites, hypersensitivity (e.g., dry skin, acne, eczema, psoriasis), inflammatory conditions or injury;

c) itch associated with vulvar vestibulitis; and

d) skin irritation or inflammatory effect from administration of another therapeutic such as, for example, antibiotics, antivirals and antihistamines.

The compounds of the invention are also useful in treating or preventing certain hormone sensitive cancers, such as prostate cancer (adenocarcinoma), breast cancer, ovarian cancer, testicular cancer, thyroid neoplasia, in a mammal, preferably a human. The voltage gated sodium channels have been demonstrated to be expressed in prostate and breast cancer cells. Up-regulation of neonatal Na(v)1.5 occurs as an integral part of the metastatic process in human breast cancer and could serve both as a novel marker of the metastatic phenotype and a therapeutic target (Clin. Cancer Res. 2005, Aug. 1; 11(15): 5381-9). Functional expression of voltage-gated sodium channel alpha-subunits, specifically Naᵥ1.7, is associated with strong metastatic potential in prostate cancer (CaP) in vitro. Voltage-gated sodium channel alpha-subunits immunostaining, using antibodies specific to the sodium channel alpha subunit was evident in prostatic tissues and markedly stronger in CaP vs non-CaP patients (Prostate Cancer Prostatic Dis., 2005; 8(3):266-73)

The compounds of the invention are also useful in treating or preventing symptoms in a mammal associated with BPH such as, but not limited to, acute urinary retention and urinary tract infection.

The compounds of the invention are also useful in treating or preventing certain endocrine imbalances or endocrinopathies such as congenital adrenal hyperplasia, hyperthyroidism, hypothyroidism, osteoporosis, osteomalacia, rickets, Cushing's Syndrome, Conn's syndrome, hyperaldosteronism, hypogonadism, hypergonadism, infertility, fertility and diabetes.

The present invention readily affords many different means for identification of sodium channel modulating agents that are useful as therapeutic agents. Identification of modulators of sodium channel can be assessed using a variety of in vitro and in vivo assays, e.g. measuring current, measuring membrane potential, measuring ion flux, (e.g. sodium or guanidinium), measuring sodium concentration, measuring second messengers and transcription levels, and using e.g., voltage-sensitive dyes, radioactive tracers, and patch-clamp electrophysiology.

One such protocol involves the screening of chemical agents for ability to modulate the activity of a sodium channel thereby identifying it as a modulating agent.

A typical assay described in Bean et al., J. General Physiology (1983), 83:613-642, and Leuwer, M., et al., Br. J. Pharmacol (2004), 141(1):47-54, uses patch-clamp techniques to study the behaviour of channels. Such techniques are known to those skilled in the art, and may be developed, using current technologies, into low or medium throughput assays for evaluating compounds for their ability to modulate sodium channel behaviour.

A competitive binding assay with known sodium channel toxins such as tetrodotoxin, alpha-scorpion toxins, aconitine, BTX and the like, may be suitable for identifying potential therapeutic agents with high selectivity for a particular sodium channel. The use of BTX in such a binding assay is well known and is described in McNeal, E. T., et al., J. Med. Chem. (1985), 28(3):381-8; and Creveling, C. R., et al., Methods in Neuroscience, Vol. 8: Neurotoxins (Conn P M Ed) (1992), pp. 25-37, Academic Press, New York.

These assays can be carried out in cells, or cell or tissue extracts expressing the channel of interest in a natural endogenous setting or in a recombinant setting. The assays that can be used include plate assays which measure Na+ influx through surrogate markers such as ¹⁴C-guanidine influx or determine cell depolarization using fluorescent dyes such as the FRET based and other fluorescent assays or a radiolabelled binding assay employing radiolabelled aconitine, BTX, TTX or STX. More direct measurements can be made with manual or automated electrophysiology systems. The guanidine influx assay is explained in more detail below in the Biological Assays section.

Throughput of test compounds is an important consideration in the choice of screening assay to be used. In some strategies, where hundreds of thousands of compounds are to be tested, it is not desirable to use low throughput means. In other cases, however, low throughput is satisfactory to identify important differences between a limited number of compounds. Often it will be necessary to combine assay types to identify specific sodium channel modulating compounds.

Electrophysiological assays using patch clamp techniques is accepted as a gold standard for detailed characterization of sodium channel compound interactions, and as described in Bean et al., op. cit. and Leuwer, M., et al., op. cit. There is a manual low-throughput screening (LTS) method which can compare 2-10 compounds per day; a recently developed system for automated medium-throughput screening (MTS) at 20-50 patches (i.e. compounds) per day; and a technology from Molecular Devices Corporation (Sunnyvale, Calif.) which permits automated high-throughput screening (HTS) at 1000-3000 patches (i.e. compounds) per day.

One automated patch-clamp system utilizes planar electrode technology to accelerate the rate of drug discovery. Planar electrodes are capable of achieving high-resistance, cells-attached seals followed by stable, low-noise whole-cell recordings that are comparable to conventional recordings. A suitable instrument is the PatchXpress 7000A (Axon Instruments Inc, Union City, Calif.). A variety of cell lines and culture techniques, which include adherent cells as well as cells growing spontaneously in suspension are ranked for seal success rate and stability. Immortalized cells (e.g. HEK and CHO) stably expressing high levels of the relevant sodium ion channel can be adapted into high-density suspension cultures.

Other assays can be selected which allow the investigator to identify compounds which block specific states of the channel, such as the open state, closed state or the resting state, or which block transition from open to closed, closed to resting or resting to open. Those skilled in the art are generally familiar with such assays.

Binding assays are also available, however these are of only limited functional value and information content. Designs include traditional radioactive filter based binding assays or the confocal based fluorescent system available from Evotec OAI group of companies (Hamburg, Germany), both of which are HTS.

Radioactive flux assays can also be used. In this assay, channels are stimulated to open with veratridine or aconitine and held in a stabilized open state with a toxin, and channel blockers are identified by their ability to prevent ion influx. The assay can use radioactive ²²[Na] and ¹⁴[C]guanidinium ions as tracers. FlashPlate & Cytostar-T plates in living cells avoids separation steps and are suitable for HTS. Scintillation plate technology has also advanced this method to HTS suitability. Because of the functional aspects of the assay, the information content is reasonably good.

Yet another format measures the redistribution of membrane potential using the FLIPR system membrane potential kit (HTS) available from Molecular Dynamics (a division of Amersham Biosciences, Piscataway, N.J.). This method is limited to slow membrane potential changes. Some problems may result from the fluorescent background of compounds. Test compounds may also directly influence the fluidity of the cell membrane and lead to an increase in intracellular dye concentrations. Still, because of the functional aspects of the assay, the information content is reasonably good.

Sodium dyes can be used to measure the rate or amount of sodium ion influx through a channel. This type of assay provides a very high information content regarding potential channel blockers. The assay is functional and would measure Na+ influx directly. CoroNa Red, SBFI and/or sodium green (Molecular Probes, Inc. Eugene Oreg.) can be used to measure Na influx; all are Na responsive dyes. They can be used in combination with the FLIPR instrument. The use of these dyes in a screen has not been previously described in the literature. Calcium dyes may also have potential in this format.

In another assay, FRET based voltage sensors are used to measure the ability of a test compound to directly block Na influx. Commercially available HTS systems include the VIPR™ II FRET system (Aurora Biosciences Corporation, San Diego, Calif., a division of Vertex Pharmaceuticals, Inc.) which may be used in conjunction with FRET dyes, also available from Aurora Biosciences. This assay measures sub-second responses to voltage changes. There is no requirement for a modifier of channel function. The assay measures depolarization and hyperpolarizations, and provides ratiometric outputs for quantification. A somewhat less expensive MTS version of this assay employs the FLEXstation™ (Molecular Devices Corporation) in conjunction with FRET dyes from Aurora Biosciences. Other methods of testing the compounds disclosed herein are also readily known and available to those skilled in the art.

These results provide the basis for analysis of the structure-activity relationship (SAR) between test compounds and the sodium channel. Certain substituents on the core structure of the test compound tend to provide more potent inhibitory compounds. SAR analysis is one of the tools those skilled in the art may now employ to identify preferred embodiments of the compounds of the invention for use as therapeutic agents.

Modulating agents so identified are then tested in a variety of in vivo models so as to determine if they alleviate pain, especially chronic pain or other conditions such as arrhythmias and epilepsy, benign prostatic hyperplasia (BPH), hypercholesterolemia, cancer and pruritis (itch) with minimal adverse events. The assays described below in the Biological Assays Section are useful in assessing the biological activity of the instant compounds.

Typically, a successful therapeutic agent of the present invention will meet some or all of the following criteria. Oral availability should be at or above 20%. Animal model efficacy is less than about 0.1 μg to about 100 mg/Kg body weight and the target human dose is between 0.1 μg to about 100 mg/Kg body weight, although doses outside of this range may be acceptable (“mg/Kg” means milligrams of compound per kilogram of body mass of the subject to whom it is being administered). The therapeutic index (or ratio of toxic dose to therapeutic dose) should be greater than 100. The potency (as expressed by IC₅₀ value) should be less than 10 μM, preferably below 1 μM and most preferably below 50 nM. The IC₅₀ (“Inhibitory Concentration—50%”) is a measure of the amount of compound required to achieve 50% inhibition of ion flux through a sodium channel, over a specific time period, in an assay of the invention. Compounds of the present invention in the guanidine influx assay have demonstrated IC-50s ranging from less than a nanomolar to less than 10 micromolar.

In an alternative use of the invention, the compounds of the invention can be used in in vitro or in vivo studies as exemplary agents for comparative purposes to find other compounds also useful in treatment of, or protection from, the various diseases disclosed herein.

Another aspect of the invention relates to inhibiting Naᵥ1.1, Naᵥ1.2, Naᵥ1.3, Naᵥ1.4, Naᵥ1.5, Naᵥ1.6, Naᵥ1.7, Naᵥ1.8, or Naᵥ1.9 activity in a biological sample or a mammal, preferably a human, which method comprises administering to the mammal, preferably a human, or contacting said biological sample with a compound of formula I or a composition comprising said compound. The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of Naᵥ1.1, Naᵥ1.2, Naᵥ1.3, Naᵥ1.4, Naᵥ1.5, Naᵥ1.6, Naᵥ1.7, Naᵥ1.8, or Naᵥ1.9 activity in a biological sample is useful for a variety of purposes that are known to one of skill in the art. Examples of such purposes include, but are not limited to, the study of sodium ion channels in biological and pathological phenomena; and the comparative evaluation of new sodium ion channel inhibitors.

The compounds of the invention, as set forth above in the Summary of the Invention, as stereoisomers, enantiomers, tautomers thereof or mixtures thereof, or pharmaceutically acceptable salts, solvates or prodrugs thereof, and/or the pharmaceutical compositions described herein which comprise a pharmaceutically acceptable excipient and one or more compounds of the invention, as set forth above in the Summary of the Invention, as a stereoisomer, enantiomer, tautomer thereof or mixtures thereof, or a pharmaceutically acceptable salt, N-oxide, solvate or prodrug thereof, can be used in the preparation of a medicament for the treatment of sodium channel-mediated disease or condition in a mammal.

### Pharmaceutical Compositions of the Invention and Administration

The present invention also relates to pharmaceutical composition containing the compounds of the invention disclosed herein. In one embodiment, the present invention relates to a composition comprising compounds of the invention in a pharmaceutically acceptable carrier, excipient or diluent and in an amount effective to modulate, preferably inhibit, ion flux through a voltage-dependent sodium channel to treat sodium channel mediated diseases, such as pain, when administered to an animal, preferably a mammal, most preferably a human patient.

Administration of the compounds of the invention, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see The Science and Practice of Pharmacy, 20th Edition (Philadelphia College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings of this invention.

The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids, such as water, saline, glycerol and ethanol, and the like. A thorough discussion of pharmaceutically acceptable carriers, diluents, and other excipients is presented in REMINGTON′S PHARMACEUTICAL SCIENCES (Mack Pub. Co., N.J. current edition).

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer.

In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained. Typically, this amount is at least 0.01% of a compound of the invention in the composition. When intended for oral administration, this amount may be varied to be between 0.1 and about 70% of the weight of the composition. Preferred oral pharmaceutical compositions contain between about 4% and about 50% of the compound of the invention. Preferred pharmaceutical compositions and preparations according to the present invention are prepared so that a parenteral dosage unit contains between 0.01 to 10% by weight of the compound prior to dilution of the invention.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the invention from about 0.1 to about 10% w/v (weight per unit volume).

The pharmaceutical composition of the invention may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the invention may be prepared by methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 kg mammal) from about 0.001 mg/kg (i.e., 0.07 mg) to about 100 mg/kg (i.e., 7.0 gm); preferably a therapeutically effective dose is (for a 70 kg mammal) from about 0.01 mg/kg (i.e., 7 mg) to about 50 mg/kg (i.e., 3.5 gm); more preferably a therapeutically effective dose is (for a 70 kg mammal) from about 1 mg/kg (i.e., 70 mg) to about 25 mg/kg (i.e., 1.75 gm).

The ranges of effective doses provided herein are not intended to be limiting and represent preferred dose ranges. However, the most preferred dosage will be tailored to the individual subject, as is understood and determinable by one skilled in the relevant arts. (see, e.g., Berkow et al., eds., The Merck Manual, 16^{th} edition, Merck and Co., Rahway, N.J., 1992; Goodmanetna., eds., Goodman and Cilman's The Pharmacological Basis of Therapeutics, 10^{th} edition, Pergamon Press, Inc., Elmsford, N.Y., (2001); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985); Osolci al., eds., Remington's Pharmaceutical Sciences, 18^{th} edition, Mack Publishing Co., Easton, Pa. (1990); Katzung, Basic and Clinical Pharmacology, Appleton and Lange, Norwalk, Conn. (1992)).

The total dose required for each treatment can be administered by multiple doses or in a single dose over the course of the day, if desired. Generally, treatment is initiated with smaller dosages, which are less than the optimum dose of the compound.

Thereafter, the dosage is increased by small increments until the optimum effect under the circumstances is reached. The diagnostic pharmaceutical compound or composition can be administered alone or in conjunction with other diagnostics and/or pharmaceuticals directed to the pathology, or directed to other symptoms of the pathology. The recipients of administration of compounds and/or compositions of the invention can be any vertebrate animal, such as mammals. Among mammals, the preferred recipients are mammals of the Orders Primate (including humans, apes and monkeys), Arteriodactyla (including horses, goats, cows, sheep, pigs), Rodenta (including mice, rats, rabbits, and hamsters), and Carnivora (including cats, and dogs). Among birds, the preferred recipients are turkeys, chickens and other members of the same order. The most preferred recipients are humans.

For topical applications, it is preferred to administer an effective amount of a pharmaceutical composition according to the invention to target area, e.g., skin surfaces, mucous membranes, and the like, which are adjacent to peripheral neurons which are to be treated. This amount will generally range from about 0.0001 mg to about 1 g of a compound of the invention per application, depending upon the area to be treated, whether the use is diagnostic, prophylactic or therapeutic, the severity of the symptoms, and the nature of the topical vehicle employed. A preferred topical preparation is an ointment, wherein about 0.001 to about 50 mg of active ingredient is used per cc of ointment base. The pharmaceutical composition can be formulated as transdermal compositions or transdermal delivery devices (“patches”). Such compositions include, for example, a backing, active compound reservoir, a control membrane, liner and contact adhesive. Such transdermal patches may be used to provide continuous pulsatile, or on demand delivery of the compounds of the present invention as desired.

The compositions of the invention can be formulated so as to provide quick, sustained or delayed release of the active ingredient after administration to the patient by employing procedures known in the art. Controlled release drug delivery systems include osmotic pump systems and dissolutional systems containing polymer-coated reservoirs or drug-polymer matrix formulations. Examples of controlled release systems are given in U.S. Pat. Nos. 3,845,770 and 4,326,525 and in P. J. Kuzma et al, Regional Anesthesia 22 (6): 543-551 (1997), all of which are incorporated herein by reference.

The compositions of the invention can also be delivered through intra-nasal drug delivery systems for local, systemic, and nose-to-brain medical therapies. Controlled Particle Dispersion (CPD)™ technology, traditional nasal spray bottles, inhalers or nebulizers are known by those skilled in the art to provide effective local and systemic delivery of drugs by targeting the olfactory region and paranasal sinuses.

The invention also relates to an intravaginal shell or core drug delivery device suitable for administration to the human or animal female. The device may be comprised of the active pharmaceutical ingredient in a polymer matrix, surrounded by a sheath, and capable of releasing the compound in a substantially zero order pattern on a daily basis similar to devises used to apply testosterone as described in PCT Patent No. WO 98/50016.

Current methods for ocular delivery include topical administration (eye drops), subconjunctival injections, periocular injections, intravitreal injections, surgical implants and iontophoresis (uses a small electrical current to transportionized drugs into and through body tissues). Those skilled in the art would combine the best suited excipients with the compound for safe and effective intra-occular administration.

The most suitable route will depend on the nature and severity of the condition being treated. Those skilled in the art are also familiar with determining administration methods (oral, intravenous, inhalation, sub-cutaneous, rectal etc.), dosage forms, suitable pharmaceutical excipients and other matters relevant to the delivery of the compounds to a subject in need thereof.

### Combination Therapy

The compounds of the invention may be usefully combined with one or more other compounds of the invention or one or more other therapeutic agent or as any combination thereof, in the treatment of sodium channel-mediated diseases and conditions. For example, a compound of the invention may be administered simultaneously, sequentially or separately in combination with other therapeutic agents, including, but not limited to:
opiates analgesics, e.g. morphine, heroin, cocaine, oxymorphine, levorphanol, levallorphan, oxycodone, codeine, dihydrocodeine, propoxyphene, nalmefene, fentanyl, hydrocodone, hydromorphone, meripidine, methadone, nalorphine, naloxone, naltrexone, buprenorphine, butorphanol, nalbuphine and pentazocine;
non-opiate analgesics, e.g. acetomeniphen, salicylates (e.g. aspirin);
nonsteroidal antiinflammatory drugs (NSAIDs), e.g. ibuprofen, naproxen, fenoprofen, ketoprofen, celecoxib, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin and zomepirac;
anticonvulsants, e.g. carbamazepine, oxcarbazepine, lamotrigine, valproate, topiramate, gabapentin and pregabalin;
antidepressants such as tricyclic antidepressants, e.g. amitriptyline, clomipramine, despramine, imipramine and nortriptyline;
COX-2 selective inhibitors, e.g. celecoxib, rofecoxib, parecoxib, valdecoxib, deracoxib, etoricoxib, and lumiracoxib;
alpha-adrenergics, e.g. doxazosin, tamsulosin, clonidine, guanfacine, dexmetatomidine, modafinil, and 4-amino-6,7-dimethoxy-2-(5-methane sulfonamido-1,2,3,4-tetrahydroisoquinol-2-yl)-5-(2-pyridyl)quinazoline;
barbiturate sedatives, e.g. amobarbital, aprobarbital, butabarbital, butabital, mephobarbital, metharbital, methohexital, pentobarbital, phenobartital, secobarbital, talbutal, theamylal and thiopental;
tachykinin (NK) antagonist, particularly an NK-3, NK-2 or NK-1 antagonist, e.g. (αR,9R)-7-[3,5-bis(trifluoromethyl)benzyl)]-8,9,10,11-tetrahydro-9-methyl-5-(4-methylphenyl)-7H-[1,4]diazocino[2,1-g][1,7]-naphthyridine-6-13-dione (TAK-637), 5-[[2R,3S)-2-[(1R)-1-[3,5-bis(trifluoromethylphenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S); coal-tar analgesics, in particular paracetamol; serotonin reuptake inhibitors, e.g. paroxetine, sertraline, norfluoxetine (fluoxetine desmethyl metabolite), metabolite demethylsertraline, ′3 fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine, trazodone and fluoxetine;
noradrenaline (norepinephrine) reuptake inhibitors, e.g. maprotiline, lofepramine, mirtazepine, oxaprotiline, fezolamine, tomoxetine, mianserin, buproprion, buproprion metabolite hydroxybuproprion, nomifensine and viloxazine (Vivalan®)), especially a selective noradrenaline reuptake inhibitor such as reboxetine, in particular (S,S)-reboxetine, and venlafaxine duloxetine neuroleptics sedative/anxiolytics;
dual serotonin-noradrenaline reuptake inhibitors, such as venlafaxine, venlafaxine metabolite O— desmethylvenlafaxine, clomipramine, clomipramine metabolite desmethylclomipramine, duloxetine, milnacipran and imipramine;
acetylcholinesterase inhibitors such as donepezil;
5-HT₃ antagonists such as ondansetron;
metabotropic glutamate receptor (mGluR) antagonists;
local anaesthetic such as mexiletine and lidocaine;
corticosteroid such as dexamethasone;
antiarrhythimics, e.g. mexiletine and phenyloin;
muscarinic antagonists, e.g., tolterodine, propiverine, tropsium t chloride, darifenacin, solifenacin, temiverine and ipratropium;
cannabinoids;
vanilloid receptor agonists (e.g. resinferatoxin) or antagonists (e.g. capsazepine);
sedatives, e.g. glutethimide, meprobamate, methaqualone, and dichloralphenazone;
anxiolytics such as benzodiazepines,
antidepressants such as mirtazapine,
topical agents (e.g. lidocaine, capsacin and resiniferotoxin);
muscle relaxants such as benzodiazepines, baclofen, carisoprodol, chlorzoxazone, cyclobenzaprine, methocarbamol and orphrenadine;
anti-histamines or H1 antagonists;
NMDA receptor antagonists;
5-HT receptor agonists/antagonists;
PDEV inhibitors;
Tramadol®;
cholinergic (nicotinc) analgesics;
alpha-2-delta ligands;
prostaglandin E2 subtype antagonists;
leukotriene B4 antagonists;
5-lipoxygenase inhibitors; and
5-HT₃ antagonists.

Sodium channel-mediated diseases and conditions that may be treated and/or prevented using such combinations include but not limited to, pain, central and peripherally mediated, acute, chronic, neuropathic as well as other diseases with associated pain and other central nervous disorders such as epilepsy, anxiety, depression and bipolar disease; or cardiovascular disorders such as arrhythmias, atrial fibrillation and ventricular fibrillation; neuromuscular disorders such as restless leg syndrome and muscle paralysis or tetanus; neuroprotection against stroke, neural trauma and multiple sclerosis; and channelopathies such as erythromyalgia and familial rectal pain syndrome.

As used herein “combination” refers to any mixture or permutation of one or more compounds of the invention and one or more other compounds of the invention or one or more additional therapeutic agent. Unless the context makes clear otherwise, “combination” may include simultaneous or sequentially delivery of a compound of the invention with one or more therapeutic agents. Unless the context makes clear otherwise, “combination” may include dosage forms of a compound of the invention with another therapeutic agent. Unless the context makes clear otherwise, “combination” may include routes of administration of a compound of the invention with another therapeutic agent. Unless the context makes clear otherwise, “combination” may include formulations of a compound of the invention with another therapeutic agent. Dosage forms, routes of administration and pharmaceutical compositions include, but are not limited to, those described herein.

### Kits-of-Parts

The present invention also provides kits that contain a pharmaceutical composition which includes one or more compounds of the invention. The kit also includes instructions for the use of the pharmaceutical composition for modulating the activity of ion channels, for the treatment of pain, as well as other utilities as disclosed herein. Preferably, a commercial package will contain one or more unit doses of the pharmaceutical composition. For example, such a unit dose may be an amount sufficient for the preparation of an intravenous injection. It will be evident to those of ordinary skill in the art that compounds which are light and/or air sensitive may require special packaging and/or formulation. For example, packaging may be used which is opaque to light, and/or sealed from contact with ambient air, and/or formulated with suitable coatings or excipients.

### Preparation of the Compounds of the Invention

The following Reaction Schemes illustrate methods to make compounds of the invention, i.e., compounds of formula (I), as described above in the Summary of the Invention. In particular, the following Reaction Schemes illustrate methods to make compounds of formula (I) having the following formula (Ia):

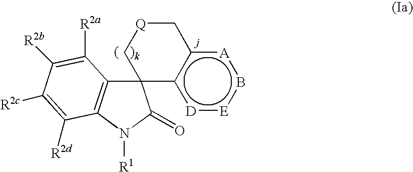

wherein j, k, Q, A, B, D, E, R¹, R^{2a}, R^{2b}, R^{2c} and R^{2d} are as defined above in the Embodiments of the Invention for compounds of formula (Ia), as a stereoisomer, enantiomer, tautomer thereof or mixtures thereof; or a pharmaceutically acceptable salt, solvate or prodrug thereof, which are used in the methods of the invention.

It is also understood that one skilled in the art would be able to make the compounds of the invention by similar methods or by methods known to one skilled in the art. It is also understood that one skilled in the art would be able to make in a similar manner as described below other compounds of the invention not specifically illustrated below, by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, e.g., Smith, M. B. and J. March, Advanced Organic Chemistry Reactions, Mechanisms, and Structure, 5th edition (Wiley, December 2000)) or prepared as described herein.

It is also understood that in the following description, combinations of substituents and/or variables of the depicted formulae are permissible only if such contributions result in stable compounds.

It will also be appreciated by those skilled in the art that in the process described below the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters.

Protecting groups may be added or removed in accordance with standard techniques, which are known to one skilled in the art and as described herein.

The use of protecting groups is described in detail in Greene, T. W. and P. G. M. Wuts, Greene's Protective Groups in Organic Synthesis (2006), 4^{th} Ed., Wiley. The protecting group may also be a polymer resin such as a Wang resin or a 2-chlorotrityl-chloride resin.

It will also be appreciated by those skilled in the art, although such protected derivatives of compounds of this invention may not possess pharmacological activity as such, they may be administered to a mammal and thereafter metabolized in the body to form compounds of the invention which are pharmacologically active. Such derivatives may therefore be described as “prodrugs”. All prodrugs of compounds of this invention are included within the scope of the invention.

In the following Reaction Schemes, j, k, Q, A, B, D, E, R¹, R^{2a}, R^{2b}, R^{2c}, R^{2d}, R^{3a}, R^{3b}, R^{3c} and R^{3d} are defined as above in the Summary of the Invention for compounds of formula (Ia), unless specifically defined otherwise; X is chloro or bromo; and R″ is an alkyl group.

### A. Preparation of Compounds of Formula (Ia), Compounds of Formula (Ib) and Compounds of Formula (Ic)

Compounds of formula (Ia-1) are compounds of formula (Ia) of the invention where A is C(R^{3a}), B is C(R^{3b}), E is N, D is C(R^{3d}), Q is —O—, j is 0 and k is 1. Compounds of formula (Ia-2) are compounds of formula (Ia) of the invention where A is C(R^{3a}), B is C(R^{3b}), E is N→O, D is C(R^{3d}), Q is —O—, j is 0 and k is 1. Compounds of formula (Ia-3) are compounds of formula (Ia) of the invention where A is C(R^{3a}), B is C(R^{3b}), E is N(H), D is C(O), Q is —O—, j is 0 and k is 1. These compounds can be synthesized following the general procedure as described below in Reaction Scheme 1, where Pg represents an oxygen-protecting group:

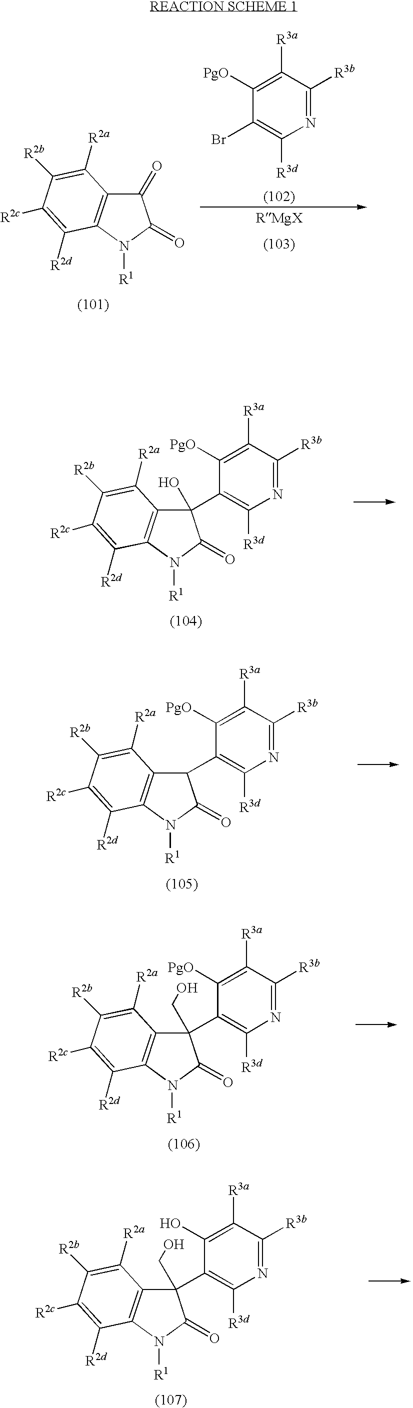

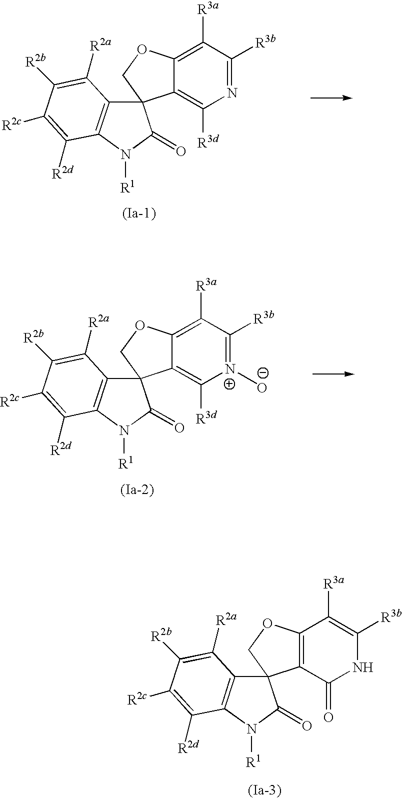

Compounds of formula (101), formula (102) and formula (103) are commercially available or can be prepared by methods known to one skilled in the art.

In general, compounds of formula (Ia-1), compounds of formula (Ia-2) and compounds of formula (Ia-3) are prepared by the procedure set forth above in Reaction Scheme 1 by first treating the bromo compound of formula (102) with a Grignard reagent (103) at low temperature (0° C.) to allow the metal-halogen exchange to take place in order to form an anion that reacts with the keto-carbonyl group of the isatin compound of formula (101) in a solvent, such as, but not limited to, tetrahydrofuran, to afford the compound oxindole of formula (104). The removal of the hydroxyl group at the C-3 position of the compound of formula (104) can be achieved by treating the compound of formula (104) with a silane reagent, such as triethylsilane, in the presence of an acid, such as, but not limited to, trifluoroacetic acid. The removal of the hydroxyl group at the C-3 position of the compound of formula (104) can also be achieved by treating the compound of formula (104) with SOCl₂/NEt₃, followed by reduction with Zn dust to give a compound of formula (105). The compound of formula (105) is then treated with a base, such as, but not limited to, diisopropylamine, lithium diisopropylamide or sodium hydroxide, followed by reaction with formaldehyde to generate the hydroxymethyl intermediate compound of formula (106). Intramolecular cyclization of the compound of formula (107) via Mitsunobu reaction conditions, such as the employment of a phosphine reagent, such as, but not limited to, triphenylphosphine or tributylphosphine, and an azo reagent, such as, but not limited to, diethyl azodicarboxylate, diisopropyl azodicarboxylate or di-tert-butyl azodicarboxylate, in a solvent, such as, but not limited to, tetrahydrofuran, dichloromethane or ethyl acetate, affords a compound of formula (Ia-1). Furthermore, treatment of compound (Ia-1) with an oxidant, such as, but not limited to, m-chloroperoxybenzoic acid, provides the oxide compound of formula (Ia-2). Reaction of compound of formula (Ia-2) (where R^{3d} is H) with an anhydride, such as, but not limited to, trifluoroacetic anhydride, leads to, via rearrangement, the formation of the pyridone compound of formula (Ia-3), which is isolated from the reaction mixture by standard isolation techniques.

### B. Preparation of Compounds of Formula (Ia-4), Compounds of Formula (Ia-5), Compounds of Formula (Ia-6), Compounds of Formula (Ia-7) and Compounds of Formula (Ia-8)

Compounds of formula (Ia-4) are compounds of formula (Ia) of the invention where A is C(R^{3a}), B is C(R^{3b}), E is C(R^{3e}), D is N, Q is —O—, j is 0 and k is 1. Compounds of formula (Ia-5) are compounds of formula (Ia) of the invention where A is C(R^{3a}), B is C(R^{3b}), E is C(R^{3e}), D is N→O, Q is —O—, j is 0 and k is 1. Compounds of formula (Ia-6) are compounds of formula (I) of the invention where R^{2a} is H, A is C(R^{3a}), B is C(R^{3b}), E is C(R^{3e}), D is N, Q is —O—, j is 0 and k is 1. Compounds of formula (Ia-7) are compounds of formula (Ia) of the invention where R^{2a} is aryl, A is C(R^{3a}), B is C(R^{3b}), E is C(R^{3e}), D is N, Q is —O—, j is 0 and k is 1. Compounds of formula (Ia-8) are compounds of formula (Ia) of the invention where A is C(R^{3a}), B is C(R^{3b}), E is C(O), D is N, Q is —O—, j is 0 and k is 1. They can be synthesized following the general procedure as described below in Reaction Scheme 2 where Ar is aryl.

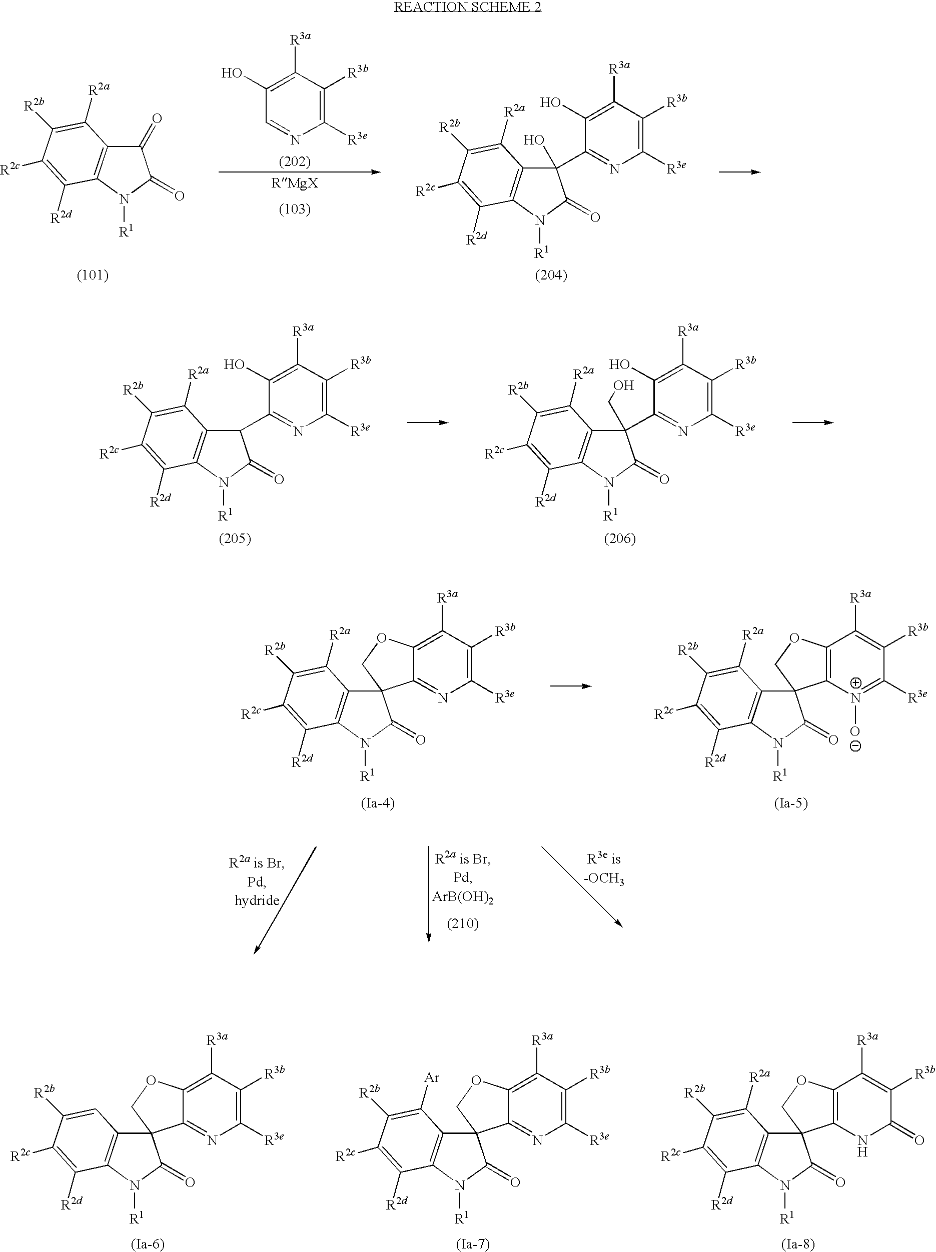

Compounds of formula (101) and (103) are commercially available or can be prepared according to one skilled in the art.

In general, compounds of formula (Ia-4), compounds of formula (Ia-5), compounds of formula (Ia-6), compounds of formula (Ia-7) and compounds of formula (Ia-8) are prepared by first treating a compound of formula (202) with a Grignard reagent (103) at low temperature (0° C.) to form the pyridyloxymagnesium halide intermediate which reacts with the keto-carbonyl group of the isatin compound of formula (101) in a solvent, such as, but not limited to, methylene chloride, tetrahydrofuran or toluene to afford the oxindole compound of formula (204). The removal of the hydroxyl group at C-3 position of the compound of formula (204) can be achieved by treating the compound of formula (204) with a silane reagent, such as triethylsilane, in the presence of an acid, such as, but not limited to, trifluoroacetic acid. The removal of the hydroxyl group at C-3 position of the compound of formula (204) can also be achieved by treating the compound (204) with SOCl₂/NEt₃, followed by reduction with Zn dust to give a compound of formula (205). A compound of formula (205) is treated with a base, such as, but not limited to, diisopropylamine, lithium diisopropylamide or sodium hydroxide, followed by reaction with formaldehyde to generate the hydroxymethyl intermediate compound of formula (206). Intramolecular cyclization of a compound of formula (206) via Mitsunobu reaction conditions, such as employment of a phosphine reagent, such as, but not limited to, triphenylphosphine or tributylphosphine, and an azo reagent, such as, but not limited to, diethyl azodicarboxylate, diisopropyl azodicarboxylate or di-tert-butyl azodicarboxylate, in a solvent, such as, but not limited to, tetrahydrofuran, dichloromethane or ethyl acetate, affords a compound of formula (Ia-4).

Furthermore, treatment of a compound of formula (Ia-4) with an oxidant, such as, but not limited to, m-chloroperoxybenzoic acid, provides an oxide compound of formula (Ia-5).

A compound of formula (Ia-4) where R^{2a} is a bromo group can be further treated under hydrogenolysis conditions, such as the employment of a palladium catalyst, such as, but not limited to, tetrakis(triphenylphosphine)palladium(0), with a hydride source, such as, but not limited to, formic acid and triethylamine, to remove the bromo group in the compound of formula (Ia-4) to produce a compound of formula (Ia-6).

Furthermore, reaction of a compound of formula (Ia-4), where R^{2a} is a bromo group, with an arylboronic acid (210) in the presence of a palladium catalyst, such as, but not limited to, palladium acetate, tetrakis(triphenylphosphine)palladium(0), or tris(dibenzylideneacetone)dipalladium(0), with or without a ligand such as, but not limited to, triphenylphosphine, tri(o-tolyl)phosphine, 1,1′-bis(diphenylphosphino)ferrocene or 2-(di-tert-butylphosphino)biphenyl, and a base such as, but not limited to, sodium carbonate, cesium carbonate, or sodium bicarbonate, in a solvent, such as, but not limited to, dimethoxyethane, dioxane, or tetrahedrofuran, provides the compound of formula (Ia-7) (see Kotha, S., et al., Tetrahedron (2002), 58:9633 and Miyaura, N., et al., Chem. Rev. (1995), 95:2457).

In addition, for compounds of formula (Ia-4) where R^{3e} is a methoxy group, a demethylation process can be conducted using a method, such as, but not limited to, treatment with TMSCl/NaI/H₂O/CH₃CN, to give the pyridone compound of formula (Ia-8).

### C. Preparation of Compounds of Formula (Ia-9) and Compounds of Formula (Ia-10)

Compounds of formula (Ia-9) and compounds of formula (Ia-10) are compounds of formula (Ia) of the invention where A is C(R^{3a}), B is C(R^{3b}), E is C(R^{3e}), D is N, Q is —O—, j is 0 and k is 1. They can be synthesized following the general procedure as described below in Reaction Scheme 3 where w and w are each independently 1, 2 or 3 and Pg¹ is a nitrogen-protecting group.

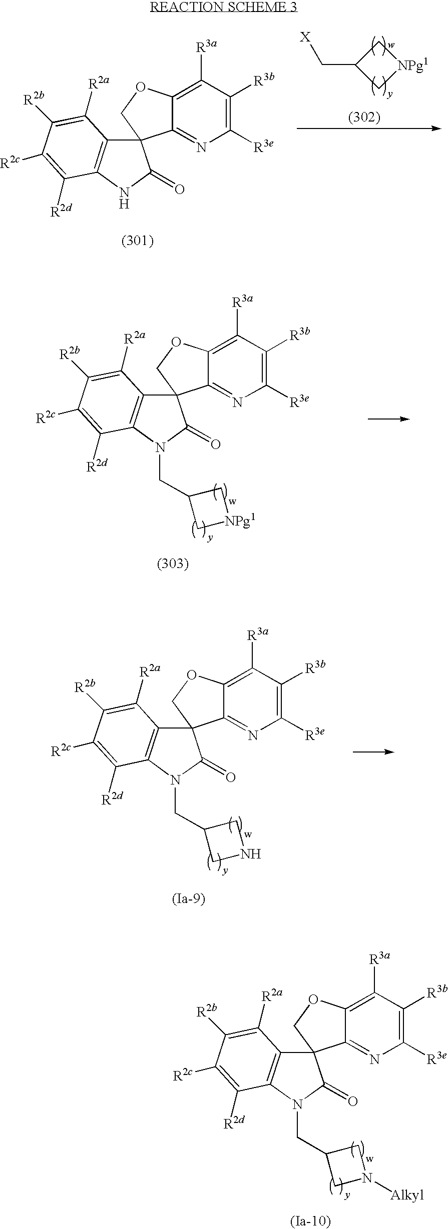

Compounds of formula (301) are compounds of formula (Ia-4) where R¹ is H. Compounds of formula (302) are commercially available or can be prepared according to methods known to one skilled in the art.

In general, compounds of formula (Ia-9) and compounds of formula (Ia-10) are prepared by the procedure set forth above in Reaction Scheme 3 by first treating a compound of formula (301) with a base such as, but not limited to, sodium hydride, cesium carbonate, or potassium carbonate, in a solvent such as, but not limited to, N,N-dimethylformamide, tetrahydrofuran, acetonitrile, or acetone, with the compounds of formula (302), where X is bromo or p-toluenesulfonate. After the removal of the nitrogen-protecting group in the compounds of formula (303) by standard procedures, the compounds of formula (Ia-9) can be obtained. Reductive amination of (Ia-9) under standard procedures provides the compounds of formula (Ia-10).

### D. Preparation of Compounds of Formula (Ia-11)

Compounds of formula (Ia-11) are compounds of formula (Ia) of the invention where A is C(R^{3a}), B is N, E is C(R^{3e}), D is C(R^{3d}), Q is —O—, j is 0 and k is 1. They can be synthesized following the general procedure as described below in Reaction Scheme 4 where Pg is an oxygen-protecting group:

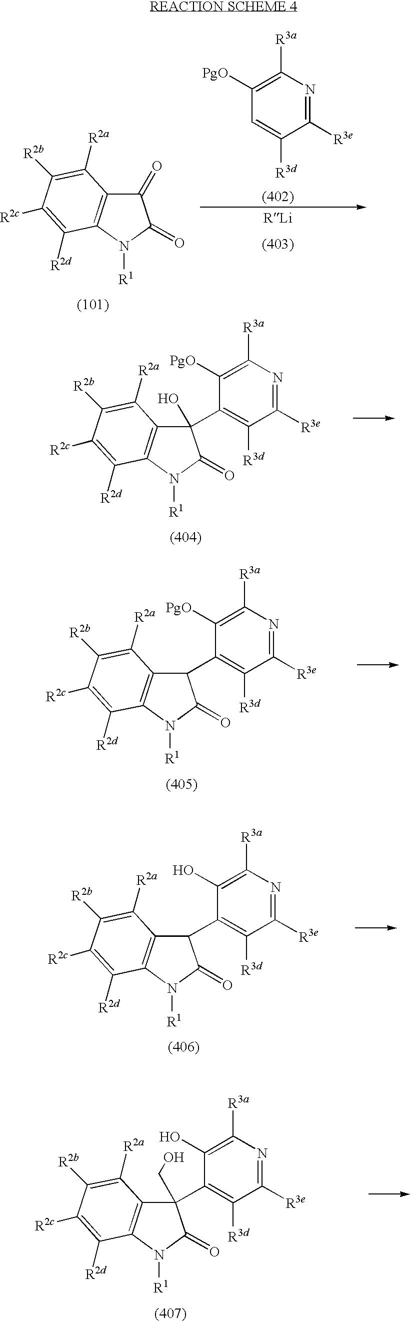

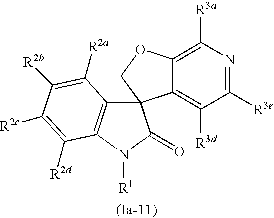

Compounds of formula (101), formula (402) and formula (403) are commercially available or can be prepared by methods known to one skilled in the art.

In general, compounds of formula (Ia-11) are prepared by the procedure set forth above in Reaction Scheme 4 by first treating the protected hydroxypyridine compound of formula (402) with a strong base (403) such as, but not limited to, tert-butyl lithium, at low temperature (−78° C.) to allow the deprotonation to take place in order to form an anion that reacts with the keto-carbonyl group of the isatin compound of formula (101) in a solvent, such as, but not limited to, tetrahydrofuran, to afford the compound oxindole of formula (404). The removal of the hydroxyl group at the C-3 position of the compound of formula (404) can be achieved by treating the compound of formula (404) with a silane reagent, such as triethylsilane, in the presence of an acid, such as, but not limited to, trifluoroacetic acid. The removal of the hydroxyl group at the C-3 position of the compound of formula (404) can also be achieved by treating the compound of formula (404) with SOCl₂/NEt₃, followed by reduction with Zn dust to give a compound of formula (405). Removal of the protecting group provides the compound of formula (406), which is then treated with a base, such as, but not limited to, diisopropylamine, lithium diisopropylamide or sodium hydroxide, followed by reaction with formaldehyde to generate the hydroxymethyl intermediate compound of formula (407). Intramolecular cyclization of the compound of formula (407) via Mitsunobu reaction conditions, such as the employment of a phosphine reagent, such as, but not limited to, triphenylphosphine or tributylphosphine, and an azo reagent, such as, but not limited to, diethyl azodicarboxylate, diisopropyl azodicarboxylate or di-tert-butyl azodicarboxylate, in a solvent, such as, but not limited to, tetrahydrofuran, dichloromethane or ethyl acetate, affords a compound of formula (Ia-11).

E. Preparation of Compounds of Formula (Ia-12), Compounds of Formula (Ia-13) and Compounds of formula (Ia-14)

Compounds of formula (Ia-12), compounds of formula (Ia-13) and compounds of formula (Ia-14) are compounds of formula (Ia) of the invention where A is C(R^{3a}), B is N, E is C(R^{3e}), D is C(R^{3d}), Q is —O—, j is 0 and k is 1. They can be synthesized following the general procedure as described below in Reaction Scheme 5 where Pg is an oxygen-protecting group and Pg¹ is an nitrogen-protecting group:

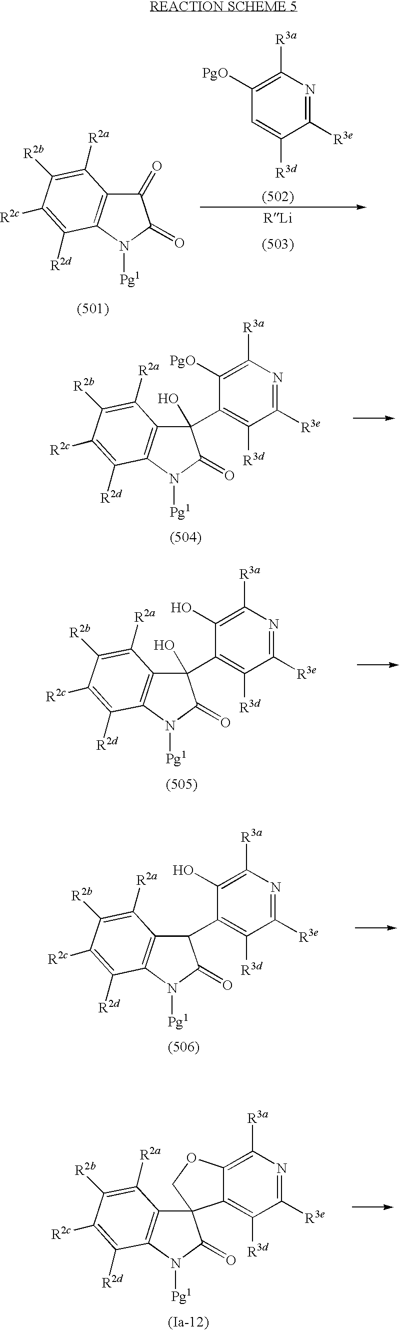

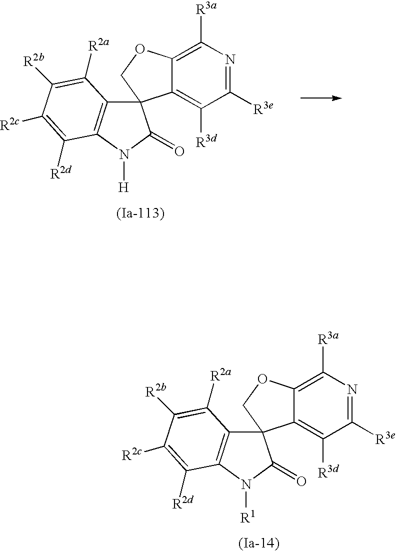

Compounds of formula (501), formula (502) and formula (503) are commercially available or can be prepared by methods known to one skilled in the art.

In general, compounds of formula (Ia-12) are prepared by the procedure set forth above in Reaction Scheme 5 by first treating the protected hydroxypyridine compound of formula (502) with a strong base (503) such as, but not limited to, tert-butyl lithium, at low temperature (−78° C.) to allow the deprotonation to take place in order to form an anion that reacts with the keto-carbonyl group of the isatin compound of formula (501) in a solvent, such as, but not limited to, tetrahydrofuran, to afford the oxindole compound of formula (504). When the oxygen-protecting group (Pg) is a methoxymethyl group, it can be removed under acidic conditions to provide the compound of formula (505). Treatment of the compound of formula (505) with SOCl₂/NEt₃, followed by reduction with Zn dust gives a compound of formula (506), which is then treated with a base, such as, but not limited to, cesium carbonate, and a dihalo reagent, such as, but not limited to, chloroiodomethane, to generate the spirooxindole compound of formula (Ia-12). When the nitrogen-protecting group (Pg¹) of the compound of formula (Ia-12) is a diphenylmethyl group, it can be removed with an acid such as, but not limited to, trifluoroacetic acid, and a silane reagent such as, but not limited to, triethylsilane, to provide compound of formula (Ia-13). Treatment of compound of formula (Ia-13) with a base such as, but not limited to, cesium carbonate or sodium hydride, in a solvent such as, but not limited to, tetrahydrofuran, N,N-dimethylformamide or acetone, and an electrophile (R¹X) affords a compound of formula (Ia-14).

All of the compounds described above as being prepared which may exist in free base or acid form may be converted to their pharmaceutically acceptable salts by treatment with the appropriate inorganic or organic base or acid. Salts of the compounds prepared above may be converted to their free base or acid form by standard techniques. It is understood that all polymorphs, amorphous forms, anhydrates, hydrates, solvates and salts of the compounds of formula (I) are intended to be within the scope of the invention. Furthermore, all compounds of formula (I) which contain an acid or an ester group can be converted to the corresponding ester or acid, respectively, by methods known to one skilled in the art or by methods described herein.

The following specific Synthetic Preparations (for the preparation of starting materials and intermediates) and Synthetic Examples (for the preparation of the compounds of formula (I), particularly compounds of formula (Ia)) are provided as a guide to assist in the practice of the invention, and are not intended as a limitation on the scope of the invention. Where one or more NMR's are given for a particular compound, each NMR may represent a single stereoisomer, a non-racemic mixture of stereoisomers or a racemic mixture of the stereoisomers of the compound.

### Synthetic Preparation 1

### Synthesis of 4-(benzyloxy)-3-bromopyridine

To a stirred solution of 3-bromo-4-pyridinol (0.45 g, 2.60 mmol) in N,N-dimethylformamide (20.0 mL) was added sodium hydride (0.13 g, 3.10 mmol) at 0° C. The mixture was allowed to stir for 30 min at 0° C. before the slow addition of benzylbromide (0.38 mL, 3.10 mmol). The mixture was stirred at ambient temperature overnight and quenched with saturated ammonium chloride (50.0 mL). The mixture was extracted with ethyl acetate (3×30.0 mL). The combined organic layers was dried over anhydrous sodium sulfate and filtered. The filtrate was concentrated in vacuo. The residue was subjected to column chromatography eluted with 2% methanol in ethyl acetate to give 4-(benzyloxy)-3-bromopyridine (0.48 g, 69%): ¹H NMR (300 MHz, CDCl₃) δ 7.78 (d, J=2.3 Hz, 1H), 7.41-7.34 (m, 4H), 7.21-7.14 (m, 2H), 6.42 (d, J=7.6 Hz, 1H), 4.79 (s, 2H); MS (ES+) m/z 264.3 (M+1), 266.3 (M+1).

### Synthetic Preparation 2

### Synthesis of 1-pentyl-1H-indole-2,3-dione

To a stirred solution of isatin (12.0 g, 81.6 mmol) in N,N-dimethylformamide (120.0 mL) was added sodium hydride (4.24 g, 60% dispersion in mineral oil, 106 mmol) slowly at 0° C. The mixture was stirred at 0° C. for 30 min before the slow addition of 1-bromopentane (13.1 mL, 106 mmol). The mixture was stirred at ambient temperature overnight, poured into ice water (500.0 mL) with stirring. The mixture was extracted with ethyl acetate (3×100.0 mL). The combined organic layers was dried over anhydrous sodium sulfate and filtered. The filtrate was concentrated in vacuo to give 1-pentyl-1H-indole-2,3-dione as an orange solid (17.5 g, 99%): ¹H NMR (300 MHz, CDCl₃) δ 7.60-7.52 (m, 2H), 7.08 (ddd, J=7.6, 7.6, 0.6 Hz, 1H), 6.87 (d, J=7.6 Hz, 1H), 3.69 (t, J=7.3 Hz, 2H), 1.74-1.61 (m, 2H), 1.40-1.28 (m, 4H), 0.88 (t, J=7.0 Hz, 3H).

### Synthetic Preparation 3

### Synthesis of 3-(hydroxymethyl)-3-(4-hydroxypyridin-3-yl)-1-pentyl-1,3-dihydro-2H-indol-2-one

### A. Synthesis of 3-[4-(benzyloxy)pyridin-3-yl]-3-hydroxy-1-pentyl-1,3-dihydro-2H-indol-2-one

To a stirred solution 4-(benzyloxy)-3-bromopyridine (0.85 g, 3.20 mmol) in anhydrous tetrahydrofuran (30.0 mL) was added isopropylmagnesium chloride (1.61 mL, 2 M solution in tetrahydrofuran, 3.50 mmol) slowly at 0° C. The mixture was allowed to stir at ambient temperature for 1 h before the addition of 1-pentyl-1H-indole-2,3-dione (0.77 g, 0.54 mmol). The mixture was stirred at ambient temperature overnight and quenched with saturated ammonium chloride (50.0 mL). The mixture was extracted with ethyl acetate (3×50.0 mL). The combined organic layers was dried over anhydrous sodium sulfate and filtered. The filtrate was concentrated in vacuo. The residue was subjected to column chromatography eluted with 2% methanol in ethyl acetate to give 3-[4-(benzyloxy)pyridin-3-yl]-3-hydroxy-1-pentyl-1,3-dihydro-2H-indol-2-one (0.52 g, 40%): ¹H NMR (300 MHz, CDCl₃) δ 7.47 (d, J=7.3 Hz, 1H), 7.42 (s, 1H), 7.39-7.25 (m, 5H), 7.17-7.11 (m, 2H), 6.98 (ddd, J=7.6, 7.6, 0.8 Hz, 1H), 6.82 (d, J=7.6 Hz, 1H), 6.43 (d, J=7.3 Hz, 1H), 4.92 (s, 2H), 3.80-3.58 (m, 2H), 1.74-1.59 (m, 2H), 1.40-1.28 (m, 4H), 0.87 (t, J=7.0 Hz, 3H); ¹³C NMR (75 MHz, CDCl₃) δ 175.2, 143.0, 139.6, 137.6, 134.1, 131.0, 130.0, 129.8, 129.4, 129.1, 128.6, 127.7, 124.4, 123.0, 118.5, 108.6, 60.7, 40.2, 29.1, 26.9, 22.3, 14.0; MS (ES+) m/z 403.5 (M+1).

### B. Synthesis of 3-[4-(benzyloxy)pyridin-3-yl]-1-pentyl-1,3-dihydro-2H-indol-2-one

A mixture of 3-[4-(benzyloxy)pyridin-3-yl]-3-hydroxy-1-pentyl-1,3-dihydro-2H-indol-2-one (0.20 g, 0.50 mmol), triethylsilane (1.6 mL, 10.0 mmol), trifluoroacetic acid (0.74 mL, 10.0 mmol) was heated at 100° C. for two days. The mixture was diluted with ethyl acetate (100.0 mL), washed with water, dried over anhydrous sodium sulfate and filtered. The filtrate was concentrated in vacuo. The residue was subjected to column chromatography eluted with 2% methanol in ethyl acetate to give 3-[4-(benzyloxy)pyridin-3-yl]-1-pentyl-1,3-dihydro-2H-indol-2-one (0.17 g, 95%): ¹H NMR (300 MHz, CDCl₃) δ 7.44-7.11 (m, 9H), 6.95 (t, J=7.6 Hz, 1H), 6.83 (d, J=7.9 Hz, 1H), 6.50 (d, J=7.6 Hz, 1H), 4.92 (s, 2H), 4.81 (s, 1H), 3.73 (t, J=7.3 Hz, 1H), 1.75-1.61 (m, 2H), 1.40-1.27 (m, 4H), 0.87 (t, J=7.0 Hz, 1H); MS (ES+) m/z 387.5 (M+1).

### C. Synthesis of 3-[4-(benzyloxy)pyridin-3-yl]-3-(hydroxymethyl)-1-pentyl-1,3-dihydro-2H-indol-2-one

A solution of 3-[4-(benzyloxy)pyridin-3-yl]-1-pentyl-1,3-dihydro-2H-indol-2-one (0.07 g, 0.18 mmol) and paraformaldehyde (0.057 g, 1.80 mmol) in anhydrous tetrahydrofuran (15.0 mL) was degassed by bubbling through argon for 1 h. Lithium diisopropylamide (0.54 mL, freshly prepared 0.5 M solution, 0.27 mmol) was added slowly at −78° C. with stirring. The mixture was stirred at ambient temperature overnight and quenched with saturated ammonium chloride (30.0 mL). The mixture was extracted with ethyl acetate (3×50.0 mL). The combined organic layers was dried over anhydrous sodium sulfate and filtered. The filtrate was concentrated in vacuo to afford 3-[4-(benzyloxy)pyridin-3-yl]-3-(hydroxymethyl)-1-pentyl-1,3-dihydro-2H-indol-2-one (0.075 g, 100%) as a solid: MS (ES+) m/z 417.5 (M+1).

### D. Synthesis of 3-(hydroxymethyl)-3-(4-hydroxypyridin-3-yl)-1-pentyl-1,3-dihydro-2H-indol-2-one

To a solution of 3-[4-(benzyloxy)pyridin-3-yl]-3-(hydroxymethyl)-1-pentyl-1,3-dihydro-2H-indol-2-one (0.075 g, 0.18 mmol) in methanol (10.0 mL) was added 10% Pd/C (0.04 g, 0.036 mmol). The mixture was hydrogenated for 4 h under normal pressure. The reaction mixture was passed through a bed of celite. The filtrate was concentrated in vacuo. The residue was subjected to column chromatography eluted with 5% methanol in ethyl acetate to give 3-(hydroxymethyl)-3-(4-hydroxypyridin-3-yl)-1-pentyl-1,3-dihydro-2H-indol-2-one (0.03 g, 51%): MS (ES+) m/z 327.5 (M+1).

### Synthetic Preparation 4

### Synthesis of [1,3]dioxolo[4,5-b]pyridin-6-ol

### A. Synthesis of [1,3]dioxolo[4,5-b]pyridin-6-ylboronic acid

To a stirred solution of 6-bromo[1,3]dioxolo[4,5-b]pyridine (Dallacker, F. et al., Z. Naturforsch. B Anorg. Chem. Org. Chem. (1979), 34:1729) (0.32 g, 1.60 mmol) in anhydrous diethyl ether (8.0 mL) was added n-butyllithium solution (1.5 mL, 1.6 M in hexane, 2.40 mmol) at −78° C. The reaction mixture was allowed to stir at −78° C. for 1 h before the quick addition of triisopropyl borate (0.73 mL, 3.20 mmol). After stirring at −78° C. for 1 h, water (20.0 mL) was added. The mixture was stirred at ambient temperature overnight. The pH value was adjusted to 10 by adding sodium hydroxide solution (2 M) slowly. The mixture was extracted with diethyl ether (3×10.0 mL). The aqueous layer was acidified to pH 4 by adding hydrobromic acid solution (≧33% in glacial acetic acid). The mixture was extracted with ethyl acetate (3×50.0 mL). The combined organic layers was dried over anhydrous sodium sulfate and filtered. The filtrate was concentrated in vacuo. The residue was subjected to column chromatography eluted with 5% methanol in ethyl acetate to give [1,3]dioxolo[4,5-b]pyridin-6-ylboronic acid (0.15 g, 50%): MS (ES+) m/z 168.2 (M+1).

### B. Synthesis of [1,3]dioxolo[4,5-b]pyridin-6-ol

To a stirred solution of [1,3]dioxolo[4,5-b]pyridin-6-ylboronic acid (0.11 g, 0.70 mmol) in anhydrous dichloromethane (5.0 mL) was added hydrogen peroxide solution (0.22 mL, 30% w/w, 2.00 mmol) at 0° C. The mixture was allowed to stir at 0° C. for 1 h and diluted with dichloromethane (50.0 mL). The organic layer was separated, dried over anhydrous sodium sulfate and filtered. The filtrate was concentrated in vacuo. The residue was subjected to column chromatography eluted with 30% ethyl acetate in hexanes to give [1,3]dioxolo[4,5-b]pyridin-6-ol (0.05 g, 49%): ¹H NMR (300 MHz, CD₃CN) δ 7.14 (d, J=2.9 Hz, 1H), 6.71 (d, J=2.9 Hz, 1H), 5.99 (s, 2H); MS (ES+) m/z 140.1 (M+1).

### Synthetic Preparation 5

### Synthesis of 1-{[5-(trifluoromethyl)-2-furyl]methyl}-1H-indole-2,3-dione

To a stirred solution of isatin (3.0 g, 20.0 mmol) in N,N-dimethylformamide (50.0 mL) was added sodium hydride (0.88 g, 60% dispersion in mineral oil, 22.0 mmol) slowly at 0° C. The mixture was allowed to stir for 30 min at 0° C. before the slow addition of 2-(bromomethyl)-5-(trifloromethyl)furan (4.95 g, 21.0 mmol). The mixture was stirred at ambient temperature overnight, poured into ice water (200.0 mL) with stirring. The mixture was filtered, and the solid obtained, 1-{[5-(trifluoromethyl)-2-furyl]methyl}-1H-indole-2,3-dione, was dried under vacuum until the weight was constant (6.02 g, 100%): ¹H NMR (300 MHz, CDCl₃) δ 7.66-7.56 (m, 2H), 7.15 (dd, J=7.6, 7.6 Hz, 1H), 7.03 (d, J=7.9 Hz, 1H), 6.74 (d, J=3.5 Hz, 1H), 6.44 (d, J=3.5 Hz, 1H), 4.92 (s, 2H).

### Synthetic Preparation 6

### Synthesis of 3-(6-hydroxy[1,3]dioxolo[4,5-b]pyridin-5-yl)-3-(hydroxymethyl)-1-{[5-(trifluoromethyl)-2-furyl]methyl}-1,3-dihydro-2H-indol-2-one

### A. Synthesis of 3-hydroxy-3-(6-hydroxy[1,3]dioxolo[4,5-b]pyridin-5-yl)-1-{[5-(trifluoromethyl)-2-furyl]methyl}-1,3-dihydro-2H-indol-2-one

To a stirred solution of [1,3]dioxolo[4,5-b]pyridin-6-ol (0.26 g, 1.87 mmol) in anhydrous tetrahydrofuran (50.0 mL) was added isopropylmagnesium chloride (1.61 mL, 2 M solution in tetrahydrofuran, 3.54 mmol) slowly at 0° C. The mixture was allowed to stir at ambient temperature for 1 h before the addition of 1-{[5-(trifluoromethyl)-2-furyl]methyl}-1H-indole-2,3-dione (0.61 g, 0.54 mmol). The mixture was stirred at ambient temperature overnight and quenched with saturated ammonium chloride (50.0 mL). The mixture was extracted with ethyl acetate (3×50.0 mL). The combined organic layers was dried over anhydrous sodium sulfate and filtered. The filtrate was concentrated in vacuo. The residue was subjected to column chromatography eluted with 30% ethyl acetate in hexanes to give 3-hydroxy-3-(6-hydroxy[1,3]dioxolo[4,5-b]pyridin-5-yl)-1-{[5-(trifluoromethyl)-2-furyl]methyl}-1,3-dihydro-2H-indol-2-one (0.67 g, 73%): mp>250° C.; ¹H NMR (300 MHz, CD₃OD) δ 7.30 (t, J=7.6 Hz, 1H), 7.19 (d, J=7.6 Hz, 1H), 7.07-6.99 (m, 2H), 6.91 (d, J=3.5 Hz, 1H), 6.67 (s, 1H), 6.57 (d, J=3.5 Hz, 1H), 6.04-6.00 (m, 2H), 5.11-4.95 (m, 2H); ¹³C NMR (75 MHz, CD₃OD) δ 178.3, 154.4, 151.7, 148.6, 143.9, 142.4, 132.9, 131.5, 130.7, 125.1, 124.5, 114.1 (m), 110.3, 110.2, 107.5, 102.4, 78.9, 37.7; MS (ES+) m/z 457.1 (M+23).

### B. Synthesis of 3-(6-hydroxy[1,3]dioxolo[4,5-b]pyridin-5-yl)-1-{[5-(trifluoromethyl)-2-furyl]methyl}-1,3-dihydro-2H-indol-2-one

A mixture of 3-hydroxy-3-(6-hydroxy[1,3]dioxolo[4,5-b]pyridin-5-yl)-1-{[5-(trifluoromethyl)-2-furyl]methyl}-1,3-dihydro-2H-indol-2-one (0.33 g, 0.75 mmol), triethylsilane (0.60 mL, 3.74 mmol), trifluoroacetic acid (0.29 mL, 3.74 mmol) and anhydrous dichloromethane (12.0 mL) was stirred at ambient temperature for 2 h. The reaction mixture was concentrated in vacuo. The residue was triturated with ether (5.0 mL) and filtered to afford 3-(6-hydroxy[1,3]dioxolo[4,5-b]pyridin-5-yl)-1-{[5-(trifluoromethyl)-2-furyl]methyl}-1,3-dihydro-2H-indol-2-one (0.27 g, 87%): mp 161-163° C.; ¹H NMR (300 MHz, CD₃OD) δ 7.25 (t, J=7.6 Hz, 1H), 7.08 (d, J=7.6 Hz, 1H), 7.05-6.98 (m, 2H), 6.90 (d, J=3.5 Hz, 1H), 6.76 (s, 1H), 6.55 (d, J=3.5 Hz, 1H), 6.03-5.98 (m, 2H), 5.06 (s, 2H), 5.03 (s, 1H); ¹³C NMR (75 MHz, CD₃OD) δ 178.2, 154.5, 152.3, 148.9, 144.2, 141.9, 132.0, 130.4, 129.1, 125.1, 124.1, 114.1 (m), 110.2, 110.0, 106.9, 102.3, 37.7; MS (ES+) m/z 419.1 (M+1).

### C. Synthesis of 3-(6-hydroxy-11,31-dioxolo[4,5-b]pyridin-5-yl)-3-(hydroxymethyl)-1-{[5-(trifluoromethyl)-2-furyl]methyl}-1,3-dihydro-2H-indol-2-one

A mixture of 3-(6-hydroxy[1,3]dioxolo[4,5-b]pyridin-5-yl)-1-{[5-(trifluoromethyl)-2-furyl]methyl}-1,3-dihydro-2H-indol-2-one (0.32 g, 0.78 mmol) and paraformaldehyde (0.23 g, 7.8 mmol) in anhydrous tetrahydrofuran (25.0 mL) was degassed by bubbling through argon for 1 h before the slow addition of lithium diisopropylamide (3.9 mL, freshly prepared 0.5 M solution in tetrahydrofuran, 3.90 mmol) at 0° C. The mixture was stirred at ambient temperature for two hours and quenched with saturated ammonium chloride (20.0 mL). The mixture was extracted with ethyl acetate (3×50 mL). The combined organic layers was dried over anhydrous sodium sulfate and filtered. The filtrate was concentrated in vacuo to give 3-(6-hydroxy[1,3]dioxolo[4,5-b]pyridin-5-yl)-3-(hydroxymethyl)-1-{[5-(trifluoromethyl)-2-furyl]methyl}-1,3-dihydro-2H-indol-2-one as a solid: MS (ES+) m/z 449.1 (M+1).

### Synthetic Preparation 7

### Synthesis of 5-fluoro-1-{[5-(trifluoromethyl)-2-furyl]methyl}-1H-indole-2,3-dione

To a solution of 5-fluoroisatin (5.00 g, 30.3 mmol) in anhydrous N,N-dimethylformamide (50 mL) was added sodium hydride (1.74 g, 60% dispersion in mineral oil, 45.4 mmol) at 0° C. The brown reaction mixture was stirred for 30 min followed by the addition of a solution of 2-(bromomethyl)-5-(trifluormethyl)furan (7.25 g, 31.8 mmol) in anhydrous N,N-dimethylformamide (7.0 mL). The reaction mixture was stirred at ambient temperature for another 6 h and poured into wet diethyl ether (200 mL). The organic layer was separated, washed with water (5×100 mL), dried over anhydrous sodium sulfate and filtered. The filtrate was concentrated in vacuo to dryness. The residue was triturated with ether to afford 5-fluoro-1-{[5-(trifluoromethyl)-2-furyl]methyl}-1H-indole-2,3-dione (5.62 g, 59%) as an orange solid: ¹H NMR (300 MHz, DMSO-d₆) δ 7.54-7.50 (m, 1H), 7.47-7.44 (m, 1H), 7.20 (dd, J=8.7, 3.9 Hz, 1H), 7.14-7.13 (m, 1H), 6.75 (d, J=3.6 Hz, 1H), 4.99 (s, 2H); ¹³C NMR (75 MHz, DMSO-d₆) δ 182.4, 160.7, 158.5, 157.5, 153.0, 146.5, 140.4 (m), 124.3, 119.3, 114.5 (m), 112.7, 112.0, 110.5, 36.8.

### Synthetic Preparation 8

### Synthesis of 4-bromo-1-{[5-(trifluoromethyl)-2-furyl]methyl}-1H-indole-2,3-dione

Following the procedure as described in SYNTHETIC PREPARATION 7 and making non-critical variations to replace 5-fluoroisatin with 4-bromoisatin, 4-bromo-1-{[5-(trifluoromethyl)-2-furyl]methyl}-1H-indole-2,3-dione was obtained (70%) as an orange solid: ¹H NMR (300 MHz, CDCl₃) δ 7.41 (dd, J=8.0, 8.0 Hz, 1H), 7.25 (dd, J=3.3, 3.3 Hz, 1H), 7.00 (d, J=7.9 Hz, 1H), 6.74 (d, J=3.3 Hz, 1H), 6.45 (d, J=3.3 Hz, 1H), 4.93 (s, 2H); ¹³C NMR (75 MHz, CDCl₃) δ 179.9, 156.8, 151.5, 150.7, 142.2, 141.8, 138.6, 129.0, 122.0, 116.4, 112.8, 110.2, 109.3, 36.6.

### Synthetic Preparation 9

### Synthesis of 4-chloro-1-{[5-(trifluoromethyl)-2-furyl]methyl}-1H-indole-2,3-dione

Following the procedure as described in SYNTHETIC PREPARATION 7 and making non-critical variations to replace 5-fluoroisatin with 4-chloroisatin, 4-chloro-1-{[5-(trifluoromethyl)-2-furyl]methyl}-1H-indole-2,3-dione was obtained (90%) as an orange solid: mp 148-150° C.; ¹H NMR (300 MHz, DMSO-d₆) δ 7.60 (dd, J=8.1, 8.1 Hz, 1H), 7.15-7.10 (m, 3H), 6.76 (d, J=3.4 Hz, 1H), 4.99 (s, 2H); ¹³C NMR (75 MHz, DMSO-d₆) δ 179.9, 157.7, 152.9 (m), 151.6, 140.1 (d, ¹J_{CF}=167 Hz), 138.9, 131.5, 124.9, 121.2, 117.7, 114.5 (m), 110.5, 110.0, 36.9.

### Synthetic Preparation 10

### Synthesis of 4-bromo-3-(3-hydroxy-6-methoxypyridin-2-yl)-3-(hydroxymethyl)-1-{[5-(trifluoromethyl)-2-furyl]methyl}-1,3-dihydro-2H-indol-2-one

### A. Synthesis of 4-bromo-3-hydroxy-3-(3-hydroxy-6-methoxypyridin-2-yl)-1-{[5-(trifluoromethyl)-2-furyl]methyl}-1,3-dihydro-2H-indol-2-one

To a solution of 6-methoxypyridin-3-ol (Van de Poel et al., Heterocycles 2002; 57: 55-71) (1.60 g, 12.8 mmol) in anhydrous tetrahydrofuran (20.0 mL) was added a solution of isopropylmagnesium chloride (6.40 mL, 2.0 M solution in ether, 12.8 mmol) at 0° C. The reaction mixture was stirred for 30 min and the solvent was removed under reduced pressure. The colourless residue was dissolved in anhydrous dichloromethane (20.0 mL) and anhydrous tetrahydrofuran (20.0 mL) and the resulted solution was added to a solution of 4-bromo-1-{[5-(trifluoromethyl)-2-furyl]methyl}-1H-indole-2,3-dione (3.19 g, 8.53 mmol) in anhydrous tetrahydrofuran (20.0 mL). The yellow reaction mixture was stirred at ambient temperature for 2 days and quenched with saturated aqueous ammonium chloride (40 mL). The reaction mixture was extracted with dichloromethane (3×100 mL) and the combined layers was dried over anhydrous sodium sulfate and filtered. The filtrate was concentrated in vacuo to dryness. The residue was purified by silica gel column chromatography eluted with ethyl acetate:hexanes (35% to 50% gradient) to give 4-bromo-3-hydroxy-3-(3-hydroxy-6-methoxypyridin-2-yl)-1-{[5-(trifluoromethyl)-2-furyl]methyl}-1,3-dihydro-2H-indol-2-one (2.75 g, 65%) as a colourless fluffy solid: R_{f}=0.44 (ethyl acetate:hexane, 1:1); ¹H NMR (300 MHz, CDCl₃) δ 7.24 (s, 1H), 7.17-7.11 (m, 1H), 6.98 (d, J=8.8 Hz, 1H), 6.89-6.84 (m, 1H), 6.67 (s, 1H), 6.54 (d, J=8.8 Hz, 1H), 4.88 (ABq, 2H), 3.78 (s, 3H); ¹³C NMR (75 MHz, CDCl₃) δ 175.5, 156.6, 151.4, 144.7, 141.9, 141.3, 135.7, 131.2, 129.6, 128.8, 127.6, 119.8, 112.7, 112.6, 112.1, 109.5, 108.0, 77.2, 53.7, 37.1.

### B. Synthesis of 4-bromo-3-(3-hydroxy-6-methoxypyridin-2-yl)-1-{[5-(trifluoromethyl)-2-furyl]methyl}-1,3-dihydro-2H-indol-2-one

To a solution of 4-bromo-3-hydroxy-3-(3-hydroxy-6-methoxypyridin-2-yl)-1-{[5-(trifluoromethyl)-2-furyl]methyl}-1,3-dihydro-2H-indol-2-one (2.33 g, 4.68 mmol) in anhydrous dichloromethane (47.0 mL) was added triethylamine (1.42 g, 1.9 mL, 14.0 mmol) followed by thionyl chloride (1.11 g, 9.36 mmol) at 0° C. The reaction mixture was stirred for 1 h and the solvent was removed under reduced pressure. The residue was re-dissolved in tetrahydrofuran (33 mL) and acetic acid (20 mL) followed by the addition of Zn dust (3.10 g, 46.8 mmol) at 0° C. The resulted mixture was stirred at ambient temperature for 16 h and the solid was filtered-off. The filtrate was concentrated in vacuo to dryness, the residue was diluted with ethyl acetate (100 mL), washed with saturated aqueous ammonium chloride (3×25 mL), water (3×25 mL), dried over anhydrous sodium sulfate and filtered. The filtrate was concentrated in vacuo to dryness. The residue was purified by silica gel column chromatography eluted with ethyl acetate:hexane (50%) to give 4-bromo-3-(3-hydroxy-6-methoxypyridin-2-yl)-1-{[5-(trifluoromethyl)-2-furyl]methyl}-1,3-dihydro-2H-indol-2-one (1.10 g, 49%) as a colourless solid: R_{f}=0.56 (ethyl acetate:hexane, 1:1); MS (ES+) m/z 485.3 (M+1), 483.2 (M+1).

### C. Synthesis of 4-bromo-3-hydroxy-6-methoxypyridin-2-yl)-3-(hydroxymethyl)-1-{[5-(trifluoromethyl)-2-furyl]methyl}-1,3-dihydro-2H-indol-2-one

To a suspension of 4-bromo-3-(3-hydroxy-6-methoxypyridin-2-yl)-1-{[5-(trifluoromethyl)-2-furyl]methyl}-1,3-dihydro-2H-indol-2-one (0.99 g, 2.04 mmol) and paraformaldehyde (0.25 g, 8.29 mmol) in dichloromethane (35 mL) was added diisopropylamine (6.16 mmol) at 0° C. The resulted mixture was stirred at ambient temperature for 16 h and quenched with saturated aqueous ammonium chloride (40 mL). The organic layer was separated, dried over anhydrous sodium sulfate and filtered. The filtrate was concentrated in vacuo to dryness. The solid residue was triturated with ether to afford 4-bromo-3-(3-hydroxy-6-methoxypyridin-2-yl)-3-(hydroxymethyl)-1-{[5-(trifluoromethyl)-2-furyl]methyl}-1,3-dihydro-2H-indol-2-one (0.75 g, 71%) as a colourless solid: ¹H NMR (300 MHz, DMSO-d₆) δ 9.13 (s, 1H), 7.15-6.92 (m, 5H), 6.58 (d, J=8.7 Hz, 1H), 6.48 (d, J=8.7 Hz, 1H), 5.02 (q, J=9.5 Hz, 2H), 4.87-4.74 (m, 2H), 4.36-4.26 (m, 1H), 3.77 (s, 3H); ¹³C NMR (75 MHz, DMSO-d₆) δ 176.7, 155.6, 154.1 (m), 146.6, 146.0, 140.0, 139.5, 139.0, 129.8, 129.6, 127.4, 126.2, 117.8, 114.5 (m), 109.9, 109.1, 107.6, 63.3, 59.9, 53.4, 37.2.

### Synthetic Preparation 11

### Synthesis of 5-fluoro-3-(3-hydroxy-6-methoxypyridin-2-yl)-3-(hydroxymethyl)-1-{[5-(trifluoromethyl)-2-furyl]methyl}-1,3-dihydro-2H-indol-2-one

### A. Synthesis of 5-fluoro-3-hydroxy-3-p-hydroxy-6-methoxypyridin-2-yl)-1-{[5-(trifluoromethyl)-2-furyl]methyl}-1,3-dihydro-2H-indol-2-one

To a solution of 6-methoxypyridin-3-ol (1.56 g, 12.5 mmol) in anhydrous tetrahydrofuran (20.0 mL) was added a solution of isopropylmagnesium chloride (7.8 mL, 2.0 M solution in tetrahydrofuran, 15.6 mmol) at 0° C. The reaction mixture was stirred for 0.5 h and followed by the addition of a solution of 5-fluoro-1-{[5-(trifluoromethyl)-2-furyl]methyl}-1H-indole-2,3-dione (3.13 g, 10.0 mmol) in anhydrous tetrahydrofuran (10.0 mL). The yellow reaction mixture was stirred at ambient temperature for 16 h and concentrated in vacuo to dryness. The residue was dissolved in ethyl acetate (100.0 mL), washed with saturated aqueous ammonium chloride (15 mL), water (3×15 mL), brine (15 mL). The organic layer was dried over anhydrous sodium sulfate and filtered. The filtrate was concentrated in vacuo to dryness. The residue was purified by silica gel column chromatography eluted with ethyl acetate in hexanes (1:1) to give 5-fluoro-3-(3-hydroxy-6-methoxypyridin-2-yl)-3-(hydroxymethyl)-1-{[5-(trifluoromethyl)-2-furyl]methyl}-1,3-dihydro-2H-indol-2-one (3.67 g, 84%) as a yellow solid: mp 110-113° C.; ¹H NMR (300 MHz, CDCl₃) δ 7.22 (br, 1H), 6.96-6.86 (m, 3H), 6.82-6.78 (m, 1H), 6.67-6.61 (m, 1H), 6.47 (m, 1H), 6.37-6.28 (m, 1H), 5.00 (br, 1H), 4.94 (d, J=16.5 Hz, 1H), 4.70 (d, J=16.5 Hz, 1H), 3.74 (s, 3H); ¹³C NMR (75 MHz, CDCl₃) δ 176.7, 161.4, 158.2, 155.6, 151.4, 144.8, 141.6 (d), 137.9, 132.1 (m), 129.6, 120.5, 117.0, 116.1, 112.6 (m), 111.9, 109.8, 109.3, 77.2, 53.6, 37.2; MS (ES+) m/z 439.2 (M+1), 421.1 (M−17).

### B. Synthesis of 5-fluoro-3-(3-hydroxy-6-methoxypyridin-2-yl)-1-{[5-(trifluoromethyl)-2-furyl]methyl}-1,3-dihydro-2H-indol-2-one

To a solution of 5-fluoro-3-hydroxy-3-(3-hydroxy-6-methoxypyridin-2-yl)-1-{[5-(trifluoromethyl)-2-furyl]methyl}-1,3-dihydro-2H-indol-2-one (3.67 g, 8.38 mmol) in anhydrous tetrahydrofuran/anhydrous dichloromethane (1:1, 100.0 mL) was added triethylamine (1.27 g, 1.8 mL, 12.6 mmol) followed by thionyl chloride (1.09 g, 1.3 mL, 9.22 mmol) at 0° C. The reaction mixture was stirred for 1 h and concentrated in vacuo to dryness. The residue was re-dissolved in tetrahydrofuran (100 mL) and acetic acid (30 mL) followed by the addition of zinc dust (1.10 g, 16.8 mmol) at 0° C. The reaction mixture was stirred at ambient temperature for 1 h and the solid was filtered-off. After the filtrate was concentrated in vacuo, the residue was diluted with ethyl acetate (100 mL), washed with saturated aqueous ammonium chloride (3×50 mL), brine (3×25 mL), dried over anhydrous sodium sulfate and filtered. The filtrate was concentrated in vacuo to dryness. The residue was purified by silica gel column chromatography eluted with ethyl acetate in hexane (30%) to give 5-fluoro-3-(3-hydroxy-6-methoxypyridin-2-yl)-1-{[5-(trifluoromethyl)-2-furyl]methyl}-1,3-dihydro-2H-indol-2-one (2.94 g, 83%) as a pale yellow solid: ¹³C NMR (75 MHz, CDCl₃) δ 177.4, 158.0, 157.5, 151.6, 147.9, 146.4, 138.4, 137.5, 132.3, 129.5, 128.8, 128.1, 114.6, 112.6 (m), 110.8, 110.5, 109.5, 53.4, 50.1, 37.1; MS (ES+) m/z 423.2 (M+1).

### C. Synthesis of 5-fluoro-3-(3-hydroxy-6-methoxypyridin-2-yl)-3-(hydroxymethyl)-1-{[5-(trifluoromethyl)-2-furyl]methyl}-1,3-dihydro-2H-indol-2-one

To a suspension of 5-fluoro-3-(3-hydroxy-6-methoxypyridin-2-yl)-1-{[5-(trifluoromethyl)-2-furyl]methyl}-1,3-dihydro-2H-indol-2-one (2.70 g, 6.40 mmol) and paraformaldehyde (1.92 g, 64.0 mmol) in tetrahydrofuran (100.0 mL) was added a solution of lithium hydroxide monohydrate (0.81 g, 19.2 mmol) in water (2.00 mL) at 0° C. The reaction mixture was stirred at ambient temperature for 2 h and quenched with saturated aqueous ammonium chloride (40.0 mL) and extracted with ethyl acetate (3×50.0 mL). The combined organic layers was dried over anhydrous sodium sulfate and filtered. The filtrate was concentrated in vacuo to dryness. The solid residue was triturated with ether to afford 5-fluoro-3-(3-hydroxy-6-methoxypyridin-2-yl)-3-(hydroxymethyl)-1-{[5-(trifluoromethyl)-2-furyl]methyl}-1,3-dihydro-2H-indol-2-one (quantitative yield) as a gummy material: MS (ES+) m/z 453.2 (M+1).

### Synthetic Preparation 12

### Synthesis of 4-chloro-3-(3-hydroxy-6-methoxypyridin-2-yl)-3-(hydroxymethyl)-1-{[5-(trifluoromethyl)-2-furyl]methyl}-1,3-dihydro-2H-indol-2-one

### A. Synthesis of 4′-chloro-3-hydroxy-3-(3-hydroxy-6-methoxypyridin-2-yl)-1-{[5-(trifluoromethyl)-2-furyl]methyl}-1,3-dihydro-2H-indol-2-one

Following the procedure as described in SYNTHETIC PREPARATION 11A and making non-critical variations to replace 5-fluoro-1-{[5-(trifluoromethyl)-2-furyl]methyl}-1H-indole-2,3-dione with 4-chloro-1-{[5-(trifluoromethyl)-2-furyl]methyl}-1H-indole-2,3-dione, 4′-chloro-3-hydroxy-3-(3-hydroxy-6-methoxypyridin-2-yl)-1-{[5-(trifluoromethyl)-2-furyl]methyl}-1,3-dihydro-2H-indol-2-one was obtained (87%) as a pale yellow solid: mp 185-188° C.; ¹H NMR (300 MHz, DMSO-d₆) δ 9.44 (s, 1H), 7.30 (dd, J=8.0, 8.0 Hz, 1H), 7.15-7.08 (m, 3H), 7.00 (br, 1H), 6.94 (d, J=8.1 Hz, 1H), 6.69 (d, J=8.7 Hz, 1H), 6.62 (d, J=8.6 Hz, 1H), 5.02 (s, 2H), 3.71 (s, 3H); ¹³C NMR (75 MHz, DMSO-d₆) δ 174.8, 155.7, 153.6, 149.1, 145.4, 140.3, 132.8, 131.5, 130.3, 129.1, 127.7, 123.8, 114.5, 111.4, 110.9, 110.1, 108.3, 77.1, 53.7, 36.9; MS (ES+) m/z 457.3 (M+1), 455.3 (M+1).

### B. Synthesis of 4-chloro-3-(3-hydroxy-6-methoxypyridin-2-yl)-1-{[5-(trifluoromethyl)-2-furyl]methyl}-1,3-dihydro-2H-indol-2-one

Following the procedure as described in SYNTHETIC PREPARATION 11B and making non-critical variations to replace 5-fluoro-3-hydroxy-3-(3-hydroxy-6-methoxypyridin-2-yl)-1-{[5-(trifluoromethyl)-2-furyl]methyl}-1,3-dihydro-2H-indol-2-one with 4′-chloro-3-hydroxy-3-(3-hydroxy-6-methoxypyridin-2-yl)-1-{[5-(trifluoromethyl)-2-furyl]methyl}-1,3-dihydro-2H-indol-2-one, 4-chloro-3-(3-hydroxy-6-methoxypyridin-2-yl)-1-{[5-(trifluoromethyl)-2-furyl]methyl}-1,3-dihydro-2H-indol-2-one was obtained (35%) as a pale yellow solid: mp 151-152° C.; ¹H NMR (300 MHz, DMSO-d₆) δ 9.47 (br, 1H), 7.26 (dd, J=8.0, 8.0 Hz, 1H), 7.18-7.12 (m, 2H), 7.08 (d, J=7.8 Hz, 1H), 6.96 (d, J=8.1 Hz, 1H), 6.59 (d, J=3.1 Hz, 1H), 6.56 (d, J=8.7 Hz, 1H), 5.16 (s, 1H), 5.02 (ABq, J=16.6 Hz, 2H), 3.41 (s, 3H); ¹³C NMR (75 MHz, DMSO-d₆) δ 174.8; MS (ES+) m/z 441.2 (M+1), 439.2 (M+1).

### C. Synthesis of 4-chloro-3-(3-hydroxy-6-methoxypyridin-2-yl)-3-(hydroxymethyl)-1-{[5-(trifluoromethyl)-2-furyl]methyl}-1,3-dihydro-2H-indol-2-one

To a suspension of 4-chloro-3-(3-hydroxy-6-methoxypyridin-2-yl)-1-{[5-(trifluoromethyl)-2-furyl]methyl}-1,3-dihydro-2H-indol-2-one (1.25 g, 2.85 mmol) and paraformaldehyde (0.34 g, 11.4 mmol) in water/tetrahydrofuran (15.0/5.0 mL) was added a solution of sodium hydroxide (0.46 g, 11.4 mmol) in water (2.00 mL) at 0° C. The reaction mixture was stirred at ambient temperature for 30 min, quenched with 10% aqueous hydrochloric acid solution (25.0 mL) and extracted with ethyl acetate (3×50.0 mL). The combined organic layers was dried over anhydrous sodium sulfate and filtered. The filtrate was concentrated in vacuo to dryness. The residue was triturated with diethyl ether to afford 4-chloro-3-(3-hydroxy-6-methoxypyridin-2-yl)-3-(hydroxymethyl)-1-{[5-(trifluoromethyl)-2-furyl]methyl}-1,3-dihydro-2H-indol-2-one (1.25 g, 94%) as a colourless solid: mp 191-193° C.; ¹H NMR (300 MHz, DMSO-d₆) δ 9.15 (s, 1H), 7.18 (dd, J=8.0, 8.0 Hz, 1H), 7.13 (dd, J=3.3, 1.1 Hz, 1H), 6.98 (d, J=8.7 Hz, 1H), 6.92 (d, J=7.8 Hz, 1H), 6.88 (d, J=8.2 Hz, 1H), 6.58 (d, J=8.6 Hz, 1H), 6.49 (d, J=3.3 Hz, 1H), 5.02 (ABq, J=16.9 Hz, 2H), 4.88 (t, J=4.9 Hz, 1H), 4.77 (dd, J=10.4, 4.7 Hz, 1H), 4.33 (dd, J=10.4, 5.2 Hz, 1H), 3.76 (s, 3H); ¹³C NMR (75 MHz, DMSO-d₆) δ 176.8, 155.7, 154.1, 146.5, 145.9, 140.0, 139.1, 129.6, 129.3, 127.9, 127.5, 123.1, 121.3, 117.8, 114.5, 109.8, 109.1, 107.2, 59.2, 53.3, 37.3; MS (ES+) m/z 471.3 (M+1), 469.3 (M+1).

### Synthetic Preparation 13

### Synthesis of 4-bromo-3-(3-hydroxy-6-methoxypyridin-2-yl)-1,3-bis(hydroxymethyl)-1,3-dihydro-2H-indol-2-one

### A. Synthesis of 4-bromo-3-hydroxy-3-(3-hydroxy-6-methoxypyridin-2-yl)-1,3-dihydro-2H-indol-2-one

To a pale yellow solution of 6-methoxypyridin-3-ol (7.16 g, 57.2 mmol) in anhydrous tetrahydrofuran (100 mL) was added isopropylmagnesium chloride (28.6 mL, 57.2 mmol, 2.0 M solution in tetrahydrofuran) at 0° C. The reaction solution was stirred for 30 min followed by the addition of solid 4-bromoisatin (10.3 g, 45.8 mmol) in portions. The reaction mixture was stirred at ambient temperature for 2 days. The reaction was quenched with 10% aqueous hydrochloric acid solution (100 mL) and extracted with ethyl acetate (3×100 mL). The combined organic layers was dried over anhydrous sodium sulfate and filtered. The filtrate was concentrated in vacuo to dryness. The residue was triturated with ether to afford 4-bromo-3-hydroxy-3-(3-hydroxy-6-methoxypyridin-2-yl)-1,3-dihydro-2H-indol-2-one (10.7 g, 67%) as a pale yellow solid: mp 172-175° C.; ¹H NMR (300 MHz, DMSO-d₆) δ 10.61 (s, 1H), 9.41 (s, 1H), 7.10 (d, J=8.0 Hz, 2H), 6.99 (d, J=8.1 Hz, 1H), 6.81 (d, J=7.6 Hz, 1H), 6.77 (br, 1H), 6.68 (d, J=8.6 Hz, 1H), 3.77 (s, 3H); ¹³C NMR (75 MHz, DMSO-d₆) δ 176.5, 155.5, 145.9, 139.4, 131.6, 128.9, 127.2, 125.6, 118.9, 111.8, 111.1, 109.3, 53.8; MS (ES+) m/z 353.2 (M+1), 351.2 (M+1).

### B. Synthesis of 4-Bromo-3-(3-hydroxy-6-methoxypyridin-2-yl)-1,3-dihydro-2H-indol 2-one

To a solution of 4-bromo-3-hydroxy-3-(3-hydroxy-6-methoxypyridin-2-yl)-1,3-dihydro-2H-indol-2-one (4.20 g, 12.0 mmol) in anhydrous dichloromethane (100.0 mL) and anhydrous tetrahydrofuran (10.0 mL) was added triethylamine (3.64 g, 36.0 mmol) and thionyl chloride (4.28 g, 36.0 mmol) at 0° C. The reaction solution was stirred for 30 min and quenched with 10% aqueous hydrochloric acid solution (50.0 mL). The resulted mixture was extracted with ethyl acetate (3×50.0 mL). The combined organic layers was washed with saturated ammonium chloride (3×50.0 mL), brine (50.0 mL), dried over anhydrous sodium sulfate and filtered. The filtrate was concentrated in vacuo to dryness. The residue was dissolved in tetrahydrofuran (70.0 mL) and acetic acid (30.0 mL) followed by the addition of zinc dust (7.84 g, 120 mmol). The reaction mixture was stirred at ambient temperature for 3 h and filtered. The filtrate was concentrated in vacuo to dryness. The residue was dissolved in ethyl acetate (100 mL), washed with saturated ammonium chloride (3×50.0 mL), brine (50.0 mL), dried over anhydrous sodium sulfate and filtered. The residue was purified by column chromatography eluting with ethyl acetate to afford 4-bromo-3-(3-hydroxy-6-methoxypyridin-2-yl)-1,3-dihydro-2H-indol-2-one (2.58 g, 64%) as a pale yellow solid: mp 149-152° C.; ¹H NMR (300 MHz, DMSO-d₆) δ 10.61 (s, 1H), 9.34 (br, 1H), 7.15-7.06 (m, 2H), 6.99 (d, J=7.8 Hz, 1H), 6.81 (d, J=7.4 Hz, 1H), 6.55 (d, J=8.5 Hz, 1H), 4.90 (s, 1H), 3.52 (s, 3H); ¹³C NMR (75 MHz, DMSO-d₆) δ 176.2, 156.5, 147.4, 147.2, 145.7, 138.9, 130.1, 127.7, 124.7, 118.5, 109.7, 108.7, 53.3, 49.4; MS (ES+) m/z 337.2 (M+1), 335.2 (M+1).

### C. Synthesis of 4-bromo-3-(3-hydroxy-6-methoxypyridin-2-yl)-1,3-bis(hydroxymethyl)-1,3-dihydro-2H-indol-2-one

To a mixture of 4-bromo-3-(3-hydroxy-6-methoxypyridin-2-yl)-1,3-dihydro-2H-indol 2-one (1.67 g, 5.00 mmol) and para-formaldehyde (0.60 g, 20.0 mmol) in tetrahydrofuran (20.0 mL) was added an aqueous solution of sodium hydroxide (0.80 g, 20.0 mL) in water (10.0 mL) at 0° C. The reaction solution was stirred for 1 h and quenched with 10% aqueous hydrochloric acid solution (50.0 mL). The reaction mixture was extracted with ethyl acetate (3×50.0 mL). The combined organic layers was washed with saturated ammonium chloride (3×50.0 mL), brine (50.0 mL), dried over anhydrous sodium sulfate and filtered. The filtrate was concentrated in vacuo to dryness. The residue was purified by column chromatography eluting with ethyl acetate in hexane (50%) to afford 4-bromo-3-(3-hydroxy-6-methoxypyridin-2-yl)-1,3-bis(hydroxymethyl)-1,3-dihydro-2H-indol-2-one (1.83 g, 81%) as a colourless solid: mp 142-145° C.; ¹H NMR (300 MHz, DMSO-d₆) δ 9.08 (s, 1H), 7.11 (d, J=7.8 Hz, 1H), 7.05 (d, J=6.7 Hz, 1H), 7.00 (dd, J=7.8, 1.0 Hz, 1H), 6.93 (d, J=8.7 Hz, 1H), 6.55 (d, J=8.6 Hz, 1H), 6.23 (br, 1H), 5.05 (q, J=10.7 Hz, 2H), 4.64 (br, 1H), 4.52 (ABq, 2H), 3.76 (s, 3H); ¹³C NMR (75 MHz, DMSO-d₆) δ 176.8, 155.5, 147.2, 146.1, 139.5, 129.6, 129.5, 127.2, 125.8, 117.7, 109.7, 108.2, 78.3, 63.5, 63.3, 53.3; MS (ES+) m/z 419.2 (M+23), 417.2 (M+23).

### Synthetic Preparation 14

### Synthesis of 3-(5-hydroxy-2-methoxypyridin-4-yl)-3-(hydroxymethyl)-1-((5-(trifluoromethyl)furan-2-yl)methyl)indolin-2-one

### A. Synthesis of 3-hydroxy-3-[2-methoxy-5-(methoxymethoxy)pyridin-4-yl]-1-{[5-(trifluoromethyl)furan-2-yl]methyl}-1,3-dihydro-2H-indol-2-one

To a solution of 2-methoxy-5-(methoxymethoxy)pyridine (Van de Poel et al., Heterocycles 2002; 57: 55-71) (12.0 g, 71.0 mmol) in anhydrous tetrahydrofuran (250 mL) at −78° C. was added over 15 minutes a solution of t-butyllithium in pentane (1.0 M, 71.0 mL, 71.0 mmol), followed by a solution of 1-{[5-(trifluoromethyl)furan-2-yl]methyl}-1H-indole-2,3-dione (22.0 g, 74.1 mmol) in anhydrous tetrahydrofuran (80 mL). The reaction mixture was allowed to warm to ambient temperature and was stirred for 16 h. A saturated aqueous solution of ammonium chloride (50 mL) was added and the reaction mixture was stirred for 10 min. The reaction mixture was diluted with ethyl acetate (400 mL) and water (200 mL) and the phases were separated. The aqueous phase was extracted with ethyl acetate (4×100 mL). The combined organic extracts was washed with water (3×100 mL) and brine (100 mL), dried over anhydrous sodium sulfate, filtered and concentrated in vacuo. The crude product was purified by column chromatography, eluting with hexanes/ethyl acetate (2/1) to afford 3-hydroxy-3-[2-methoxy-5-(methoxymethoxy)pyridin-4-yl]-1-{[5-(trifluoromethyl)furan-2-yl]methyl}-1,3-dihydro-2H-indol-2-one (6.61 g, 20%) as an orange microcrystalline solid: ¹H NMR (300 MHz, CDCl₃) δ7.81 (s, 1H), 7.38-7.30 (m, 1H), 7.23 (s, 1H), 7.12-7.00 (m, 3H), 6.79-6.75 (m, 1H), 6.51-6.47 (m, 1H), 5.06 (d, J=16.1 Hz, 1H), 4.82 (d, J=16.1 Hz, 1H), 4.66 (d, J=6.7 Hz, 1H), 4.29 (d, J=6.7 Hz, 1H), 3.90 (s, 3H), 3.52 (br s, 1H), 2.92 (s, 3H); MS (ES+) m/z 465.2 (M+1).

### B. Synthesis of 3-[2-methoxy-5-(methoxymethoxy)pyridin-4-yl]-1-{[5-(trifluoromethyl)furan-2-yl]methyl}-1,3-dihydro-2H-indol-2-one

To a cooled (0° C.) solution of 3-hydroxy-3-[2-methoxy-5-(methoxymethoxy)pyridin-4-yl]-1-{[5-(trifluoromethyl)furan-2-yl]methyl}-1,3-dihydro-2H-indol-2-one (6.61 g, 14.2 mmol) in anhydrous dichloromethane (100 mL) was added dropwise triethylamine (6.0 mL, 43 mmol), followed by thionyl chloride (2.1 mL, 29 mmol). The reaction mixture was stirred for 1 h at 0° C. and was concentrated in vacuo. The residue was partitioned between water (100 mL) and ethyl acetate (250 mL). The organic phase was washed with brine (100 mL), dried over anhydrous sodium sulfate, filtered and concentrated in vacuo. The residue was dissolved in anhydrous tetrahydrofuran (210 mL). Glacial acetic acid (30 mL) was added, followed by zinc (10 μm powder, 9.25 g, 142 mmol). The reaction mixture was heated at reflux for 44 h and was allowed to cool to ambient temperature. The mixture was filtered through a pad of Celite and the pad was washed with ethyl acetate (250 mL). The filtrate was concentrated to dryness in vacuo and the residue was purified by column chromatography, eluting with hexanes/ethyl acetate (5/2), followed by hexanes/ethyl acetate (2/1) and hexanes/ethyl acetate (3/2) to afford 3-[2-methoxy-5-(methoxymethoxy)pyridin-4-yl]-1-{[5-(trifluoromethyl)furan-2-yl]methyl}-1,3-dihydro-2H-indol-2-one (2.97 g, 47%) as an amber foam: ¹H NMR (300 MHz, CDCl₃) δ7.95 (s, 1H), 7.37-7.28 (m, 1H), 7.08-6.92 (m, 4H), 6.78-6.72 (m, 1H), 6.56 (s, 1H), 6.46-6.41 (m, 1H), 5.09-4.62 (m, 4H), 3.87 (s, 3H), 3.15 (s, 3H); MS (ES+) m/z 449.4 (M+1).

### C. Synthesis of 3-(5-hydroxy-2-methoxypyridin-4-yl)-1-{[5-(trifluoromethyl)furan-2-yl]methyl}-1,3-dihydro-2H-indol-2-one

To a cooled (0° C.) solution of 3-[2-methoxy-5-(methoxymethoxy)pyridin-4-yl]-1-{[5-(trifluoromethyl)furan-2-yl]methyl}-1,3-dihydro-2H-indol-2-one (2.85 g, 6.35 mmol) in dichloromethane (40 mL) was added trifluoroacetic acid (10 mL). The reaction mixture was allowed to warm to ambient temperature and was stirred for 7 h. The reaction mixture was concentrated in vacuo and the residue partitioned between dichloromethane (100 mL) and a saturated aqueous solution of sodium bicarbonate (100 mL). The organic phase was washed with water (50 mL) and brine (50 mL), dried over anhydrous sodium sulfate, filtered and concentrated in vacuo. The residue was purified by column chromatography, eluting with a 15 to 70% gradient of ethyl acetate in hexanes to afford 3-(5-hydroxy-2-methoxypyridin-4-yl)-1-{[5-(trifluoromethyl)furan-2-yl]methyl}-1,3-dihydro-2H-indol-2-one (1.80 g, 70%) as a yellow foam: MS (ES+) m/z 405.3 (M+1).

### D. Synthesis of 3-(5-hydroxy-2-methoxypyridin-4-yl)-3-(hydroxymethyl)-1-{[5-(trifluoromethyl)furan-2-yl]methyl}-1,3-dihydro-2H-indol-2-one

To a cooled (0° C.) solution of diisopropylamine (0.21 mL, 1.5 mmol) in anhydrous tetrahydrofuran (5 mL) was added dropwise a solution of n-butyllithium (1.2 M, 1.2 mL, 1.4 mmol) in hexanes. The reaction mixture was stirred for 30 min at 0° C. and was then cooled to −78° C. A solution of 3-(5-hydroxy-2-methoxypyridin-4-yl)-1-{[5-(trifluoromethyl)furan-2-yl]methyl}-1,3-dihydro-2H-indol-2-one (0.27 g, 0.67 mmol) in anhydrous tetrahydrofuran (5 mL) was added and the reaction mixture was stirred for 15 min. p-Formaldehyde (0.20 g, 6.7 mmol) was added in one portion and the reaction mixture was allowed to warm to ambient temperature. After 1.5 h, the reaction mixture was diluted with a saturated aqueous solution of ammonium chloride (15 mL) and ethyl acetate (30 mL). The aqueous phase was extracted with ethyl acetate (3×20 mL). The combined organic phases were washed with brine (20 mL), dried over anhydrous sodium sulfate, filtered and concentrated in vacuo. The residue was purified by column chromatography, eluting with a 20 to 80% gradient of ethyl acetate in hexanes, to afford 3-(5-hydroxy-2-methoxypyridin-4-yl)-3-(hydroxymethyl)-1-((5-(trifluoromethyl)furan-2-yl)methyl)indolin-2-one as a yellow foam (0.23 g): MS (ES+) m/z 417 (M−17).

### Synthetic Preparation 15

### Synthesis of 1-(diphenylmethyl)-3-(5-hydroxy-2-methoxypyridin-4-yl)-1,3-dihydro-2H-indol-2-one

### A. Synthesis of 1-(diphenylmethyl)-3-hydroxy-3-[2-methoxy-5-(methoxymethoxy)pyridin-4-yl]-1,3-dihydro-2H-indol-2-one

To a solution of 2-methoxy-5-(methoxymethoxy)pyridine (Van de Poel et al., Heterocycles 2002; 57: 55-71) (6.00 g, 35.4 mmol) in anhydrous tetrahydrofuran (200 mL) at −78° C. was added over 15 minutes a solution of t-butyllithium in pentane (1.2 M, 30.0 mL, 35.4 mmol). After stirring at −78° C. for 0.5 h, 1-(diphenylmethyl)-1H-indole-2,3-dione (Schoenberg, A. et al., Chem. Ber. 1963; 96:3328-3337) (13.3 g, 42.5 mmol) was added in one portion. The reaction mixture was allowed to warm to ambient temperature and was stirred for 16 h. A saturated aqueous solution of ammonium chloride (50 mL) was added and the reaction mixture was stirred for 10 minutes. The reaction mixture was diluted with ethyl acetate (200 mL) and water (200 mL) and the phases were separated. The organic phase was washed with water (50 mL) and brine (50 mL), dried over anhydrous sodium sulfate, filtered and concentrated in vacuo. The crude product was purified by column chromatography, eluting with hexanes/ethyl acetate (5/2) to afford 1-(diphenylmethyl)-3-hydroxy-3-[2-methoxy-5-(methoxymethoxy)pyridin-4-yl]-1,3-dihydro-2H-indol-2-one (10.9 g, 64%) as a pale orange microcrystalline solid: ¹H NMR (300 MHz, CDCl₃) δ7.86 (s, 1H), 7.52-7.23 (m, 11H), 7.11-6.90 (m, 4H), 6.54 (d, J=7.8 Hz, 1H), 4.53 (d, J=6.9 Hz, 1H), 4.22 (d, J=6.9 Hz, 1H), 3.90 (s, 3H), 3.79 (br s, 1H), 2.88 (s, 3H); MS (ES+) m/z 483.1 (M+1).

### B. Synthesis of 1-(diphenylmethyl)-3-hydroxy-3-(5-hydroxy-2-methoxypyridin-4-yl)-1,3-dihydro-2H-indol-2-one

To a solution of 1-(diphenylmethyl)-3-hydroxy-3-[2-methoxy-5-(methoxymethoxy)pyridin-4-yl]-1,3-dihydro-2H-indol-2-one (10.57 g, 21.9 mmol) in dichloromethane (40 mL) was added trifluoroacetic acid (40 mL) and the reaction mixture was stirred at ambient temperature for 16 h. The reaction mixture was concentrated in vacuo to afford 1-(diphenylmethyl)-3-hydroxy-3-(5-hydroxy-2-methoxypyridin-4-yl)-1,3-dihydro-2H-indol-2-one (9.32 g, 97%) as a colorless foam: ¹H NMR (300 MHz, DMSO-d₆) δ9.40 (br s, 1H), 7.50 (s, 1H), 7.42-7.23 (m, 11H), 7.19 (s, 1H), 6.99-6.76 (m, 4H), 6.32 (d, J=7.8 Hz, 1H), 3.77 (s, 3H); MS (ES+) m/z 439.1 (M+1).

### C. Synthesis of 1-(diphenylmethyl)-3-(5-hydroxy-2-methoxypyridin-4-yl)-1,3-dihydro-2H-indol-2-one

To a cooled (0° C.) suspension of 1-(diphenylmethyl)-3-hydroxy-3-(5-hydroxy-2-methoxypyridin-4-yl)-1,3-dihydro-2H-indol-2-one (10.1 g, 23.1 mmol) in 1,2-dichloroethane was added triethylamine (9.6 mL, 69 mmol), followed by thionyl chloride (3.7 mL, 46 mmol). The reaction mixture was allowed to warm to ambient temperature over 0.5 h and was subsequently heated at reflux for 1.5 h. The reaction mixture was allowed to cool to ambient temperature and was washed with water (2×50 mL) and brine (50 mL), dried over anhydrous sodium sulfate, filtered, concentrated in vacuo and dried under high vacuum to afford a colourless foam. To a solution of the generated foam (11 g) in tetrahydrofuran (210 mL) was added glacial acetic acid (30 mL), followed by zinc dust (15.1 g, 231 mmol). The reaction mixture was stirred for 16 h at ambient temperature. Further zinc dust (7.55 g, 115 mmol) was added and the reaction mixture was heated at 50° C. for 24 h. The reaction mixture was allowed to cool to ambient temperature and was filtered through a pad of diatomaceous earth. The pad was washed with ethyl acetate (200 mL) and was concentrated in vacuo. The residue was purified by column chromatography, eluting with hexanes/ethyl acetate (2/1) followed by hexanes/ethyl acetate (1/1) to obtain 1-(diphenylmethyl)-3-(5-hydroxy-2-methoxypyridin-4-yl)-1,3-dihydro-2H-indol-2-one (3.33 g, 34%) as a colorless solid: MS (ES+) m/z 423.3 (M+1).

### Synthetic Example 1

### Synthesis of 1′-pentylspiro[furo[3,2-c]pyridine-3,3′-indol]-2′(1′H)-one

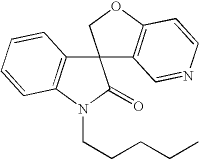

To a solution of 3-(hydroxymethyl)-3-(4-hydroxypyridin-3-yl)-1-pentyl-1,3-dihydro-2H-indol-2-one (0.03 g, 0.09 mmol) in anhydrous tetrahydrofuran (5.0 mL) was added triphenylphosphine (0.047 g, 0.18 mmol) and diethyl azodicarboxylate (0.028 mL, 0.18 mmol) at 0° C. The mixture was stirred at ambient temperature for 16 h and quenched with saturated ammonium chloride (20.0 mL). The mixture was extracted with ethyl acetate (3×30.0 mL). The combined organic layers was dried over anhydrous sodium sulfate and filtered. The filtrate was concentrated in vacuo. The residue was subjected to column chromatography eluted with 30% ethyl acetate in hexanes to give 1′-pentylspiro[furo[3,2-c]pyridine-3,3′-indol]-2′(1′H)-one (0.02 g, 71%): ¹H NMR (300 MHz, CDCl₃) δ 8.36 (d, J=5.6 Hz, 1H), 7.89 (s, 1H), 7.33 (ddd, J=7.3, 7.3, 1.8 Hz, 1H), 7.13-7.00 (m, 2H), 6.97-6.87 (m, 2H), 5.00 (d, J=9.1 Hz, 1H), 4.74 (d, J=9.1 Hz, 1H), 3.89-3.62 (m, 2H), 1.79-1.66 (m, 2H), 1.41-1.30 (m, 4H), 0.89 (t, J=7.0 Hz, 3H); ¹³C NMR (75 MHz, CDCl₃) δ 176.3, 167.2, 151.0, 145.0, 142.57, 131.6, 129.4, 123.9, 123.4, 109.0, 106.6, 80.9, 56.0, 40.5, 29.0, 27.1, 22.3, 14.0; MS (ES+) m/z 309.5 (M+1).

### Synthetic Example 2

### Synthesis of 1′-pentylspiro[furo[3,2-c]pyridine-3,3′-indol]-2′(1′H)-one 5-oxide

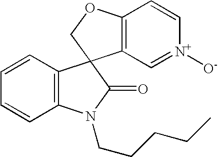

To a solution of 1′-pentylspiro[furo[3,2-c]pyridine-3,3′-indol]-2′(1′H)-one (0.045 g, 0.15 mmol) in anhydrous dichloromethane (2.0 mL) was added m-chloroperoxybenzoic acid (0.049 g, 0.22 mmol) at 0° C. The mixture was stirred at ambient temperature for 16 h and neutralized with saturated sodium bicarbonate (10.0 mL). Dichloromethane (20.0 mL) was added to the mixture. The organic layer was separated, washed with brine, dried over anhydrous sodium sulfate and filtered. The filtrate was concentrated in vacuo. The residue was subjected to column chromatography eluted with 2% methanol in ethyl acetate to afford 1′-pentylspiro[furo[3,2-c]pyridine-3,3′-indol]-2′(1′H)-one 5-oxide (0.04 g, 85%): ¹H NMR (300 MHz, CDCl₃) δ 8.10 (d, J=6.7 Hz, 1H), 7.62 (s, 1H), 7.32 (ddd, J=7.6, 7.6, 1.8 Hz, 1H), 7.18-7.05 (m, 2H), 6.97-6.86 (m, 2H), 5.10 (d, J=9.1 Hz, 1H), 4.84 (d, J=9.1 Hz, 1H), 3.86-3.59 (m, 2H), 1.79-1.66 (m, 2H), 1.41-1.30 (m, 4H), 0.89 (t, J=7.0 Hz, 3H); MS (ES+) m/z 325.5 (M+1).

### Synthetic Example 3

### Synthesis of 1′-pentylspiro[furo[3,2-c]pyridine-3,3′-indole]-2′,4(1′H,5H)-dione

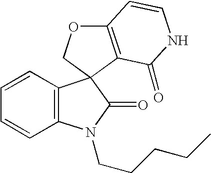

To a solution of 1′-pentylspiro[furo[3,2-c]pyridine-3,3′-indol]-2′(1′H)-one 5-oxide (0.045 g, 0.14 mmol) and triethylamine in anhydrous tetrahydrofuran (5.0 mL) was added trifluoroacetic anhydride (0.19 mL, 1.40 mmol) at 0° C. The mixture was stirred at ambient temperature for 5 h before the addition of methanol (1.0 mL) and sodium hydroxide (2 N, 1.0 mL). The mixture was stirred at ambient temperature for 16 h and quenched with 25% ammonium acetate (10.0 mL). The mixture was extracted with ethyl acetate (3×30.0 mL). The combined organic layers was dried over anhydrous sodium sulfate and filtered. The filtrate was concentrated in vacuo. The residue was subjected to column chromatography eluted with 50% ethyl acetate in hexanes to give 1′-pentylspiro[furo[3,2-c]pyridine-3,3′-indole]-2′,4(1′H,5H)-dione (0.015 g, 33%): ¹H NMR (300 MHz, CDCl₃) δ 11.64 (br, 1H), 7.30-7.22 (m, 1H), 7.09 (d, J=7.3 Hz, 1H), 7.03-6.94 (m, 2H), 6.85 (d, J=7.6 Hz, 1H), 6.03 (d, J=7.3 Hz, 1H), 4.91 (d, J=9.4 Hz, 1H), 4.67 (d, J=9.4 Hz, 1H), 3.82-3.61 (m, 2H), 1.77-1.62 (m, 2H), 1.42-1.28 (m, 4H), 0.87 (t, J=7.0 Hz, 3H); ¹³C NMR (75 MHz, CDCl₃) δ 176.1, 170.5, 161.2, 142.9, 137.5, 131.0, 128.9, 123.3, 122.9, 111.7, 108.5, 95.2, 81.9, 55.9, 40.5, 28.9, 26.9, 22.4, 14.0; MS (ES+) m/z 325.4 (M+1).

### Synthetic Example 4

### Synthesis of 4′-bromo-5-methoxy-1′-{[5-(trifluoromethyl)-2-furyl]methyl}spiro[furo[3,2-b]pyridine-3,3′-indol]-2′(1′H)-one

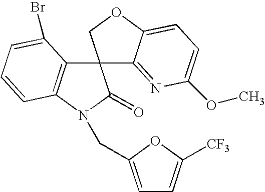

To a solution of 4-bromo-3-(3-hydroxy-6-methoxypyridin-2-yl)-3-(hydroxymethyl)-1-{[5-(trifluoromethyl)-2-furyl]methyl}-1,3-dihydro-2H-indol-2-one (0.72 g, 1.37 mmol) in ethyl acetate (25 mL) was added tributylphosphine (0.51 mL, 2.06 mmol) at 0° C., followed by the slow addition of a solution of di-tert-butyl azodicarboxylate (0.47 g, 2.05 mmol) in ethyl acetate (25 mL) over a period of 5 min. The resulted mixture was stirred at 0° C. for 10 min and quenched with saturated aqueous ammonium chloride (40 mL). The organic layer was separated, washed with hydrochloric acid (1.0 N, 3×50 mL), dried over sodium sulfate and filtered. The filtrate was concentrated in vacuo. The brown residual oil was purified by silica gel column chromatography eluted with ethyl acetate in hexanes (20-50%) to afford 4′-bromo-5-methoxy-1′-{[5-(trifluoromethyl)-2-furyl]methyl}spiro[furo[3,2-b]pyridine-3,3′-indol]-2′(1′H)-one (0.59 g, 87%) as a colourless solid: ¹H NMR (300 MHz, CDCl₃) δ 7.20-7.13 (m, 3H), 6.94-6.86 (m, 1H), 6.69 (s, 1H), 6.58 (d, J=8.8 Hz, 1H), 6.45 (s, 1H), 5.01 (ABq, J=9.5 Hz, 2H), 4.98 (ABq, J=16.6 Hz, 2H), 3.63 (s, 3H); MS (ES+) m/z 497.2 (M+1), 495.2 (M+1).

### Synthetic Example 5

### Synthesis of 5-methoxy-1′-{[5-(trifluoromethyl)-2-furyl]methyl}spiro[furo[3,2-b]pyridine-3,3′-indol]-2′(1′H)-one

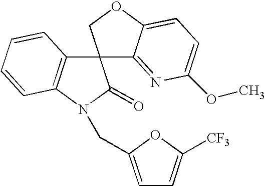

A mixture of 4′-bromo-5-methoxy-1′-{[5-(trifluoromethyl)-2-furyl]methyl}spiro[furo[3,2-b]pyridine-3,3′-indol]-2′(1′H)-one (0.25 g, 0.51 mmol), tetrakis(triphenylphosphine)palladium(0) (0.12 g, 0.10 mmol), formic acid (0.20 mL, 5.22 mmol), triethylamine (0.75 mL, 5.38 mmol) in 1,4-dioxane (7.0 mL) was heated to reflux for 16 h. After cooling down to ambient temperature, the reaction mixture was diluted with ethyl acetate (40 mL) and passed through a bed of celite. The filtrate was washed with brine (3×50 mL), dried over anhydrous sodium sulfate and filtered. The filtrate was concentrated in vacuo. The brown residual oil was purified by silica gel column chromatography eluted with ethyl acetate:hexane (20-35%) to afford 5-methoxy-1′-{[5-(trifluoromethyl)-2-furyl]methyl}spiro[furo[3,2-b]pyridine-3,3′-indol]-2′(1′H)-one (0.21 g, 98%) as a colourless solid: mp 145-147° C.; ¹H NMR (300 MHz, CD₃CN) δ 7.32 (dd, J=7.7 Hz, 1H), 7.30 (d, J=3.0 Hz, 1H), 7.18 (d, J=7.6 Hz, 1H), 7.12-7.03 (m, 2H), 6.91 (s, 1H), 6.61 (d, J=8.9 Hz, 1H), 6.56 (d, J=8.9 Hz, 1H), 4.99 (ABq, 2H), 4.85 (ABq, 2H), 3.56 (s, 3H); ¹³C NMR (75 MHz, DMSO-d₆) δ 176.0, 159.9, 153.8, 153.7, 149.7, 146.0, 142.5, 139.8 (m), 131.2, 129.4, 124.3, 123.7, 122.2, 114.5 (m), 110.8, 109.8, 109.7, 79.1, 58.5, 53.6, 37.0; MS (ES+) m/z 417.2 (M+1).

### Synthetic Example 6

### Synthesis of 4′-(3-furyl)-5-methoxy-1′-{[5-(trifluoromethyl)-2-furyl]methyl}spiro[furo[3,2-b]pyridine-3,3′-indol]-2′(1′H)-one

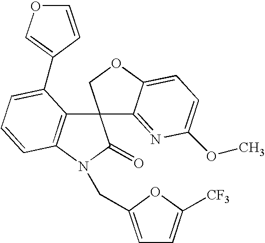

A mixture of 4′-bromo-5-methoxy-1′-{[5-(trifluoromethyl)-2-furyl]methyl}spiro[furo[3,2-b]pyridine-3,3′-indol]-2′(1′H)-one (0.072 g, 0.15 mmol), 3-furanboronic acid (0.027 g, 0.24 mmol) and tetrakis(triphenylphosphine)palladium(0) (0.026 g, 0.022 mmol) was flushed with nitrogen for 5 min. Dioxane (7.0 mL) and sodium carbonate solution (2 M, 1.0 mL) were added. The reaction mixture was heated at reflux for 16 h. After cooling down to ambient temperature, the mixture was concentrated in vacuo. The residue was extracted with ethyl acetate (4×15 mL) and the combined organic layers was passed through a pad of celite. The filtrate was concentrated in vacuo to dryness. The brown residue was purified by a silica gel column chromatography eluted with ethyl acetate:hexane (20-35%) to afford 4′-(3-furyl)-5-methoxy-1′-{[5-(trifluoromethyl)-2-furyl]methyl}spiro[furo[3,2-b]pyridine-3,3′-indol]-2′(1′H)-one (0.052 g, 74%) as a colourless solid: ¹H NMR (300 MHz, CD₃CN) δ 7.40-7.32 (m, 2H), 7.12 (d, J=8.8 Hz, 1H), 7.06 (d, J=7.8 Hz, 1H), 6.99 (d, J=7.8 Hz, 1H), 6.92 (d, J=2.9 Hz, 1H), 6.85 (s, 1H), 6.62 (d, J=8.8 Hz, 1H), 6.57 (d, J=3.2 Hz, 1H), 5.94 (s, 1H), 5.00 (ABq, 2H), 4.67 (ABq, 2H), 3.60 (s, 3H); MS (ES+) m/z 483.2 (M+1).

### Synthetic Example 7

### Synthesis of 1′-{[5-(trifluoromethyl)-2-furyl]methyl}spiro[furo[3,2-b]pyridine-3,3′-indole]-2′,5(1′H,4H)-dione

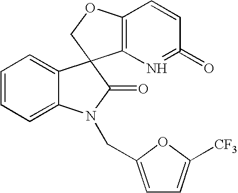

To a solution of 5-methoxy-1′((5-(trifluoromethyl)furan-2-yl)methyl)-2H-spiro[furo[3,2-b]pyridine-3,3′-indolin]-2′-one (0.20 g, 0.48 mmol) in acetonitrile (12 mL) was added chlorotrimethylsilane (0.2 mL, 1.59 mmol), catalytic amount of water (6 drops) and sodium iodide (0.20 g, 1.33 mmol). The resulted solution was stirred at 65° C. for 2 days. The reaction was quenched with 5% aqueous sodium sulfite (25 mL) and followed by the addition of brine (25 mL). The resulted mixture was extracted with ethyl acetate (3×50 mL). The combined organic layers was dried over anhydrous sodium sulfate and filtered. The filtrate was concentrated in vacuo to dryness. The residue was purified by silica gel column chromatography eluted with ethyl acetate in hexanes (40%) to afford 1′-{[5-(trifluoromethyl)-2-furyl]methyl}spiro[furo[3,2-b]pyridine-3,3′-indole]-2′,5(1′H,4H)-dione (0.12 g, 60%) as a colourless solid: mp 175-177° C.; ¹H NMR (300 MHz, CDCl₃) δ 7.38-7.26 (m, 2H), 7.14-6.96 (m, 3H), 6.70 (d, J=2.5 Hz, 1H), 6.49 (d, J=9.4 Hz, 1H), 6.44 (d, J=3.3 Hz, 1H), 4.99 (ABq, 2H), 4.88 (ABq, 2H), 3.20-2.20 (br, 1H); MS (ES+) m/z 403.2 (M+1).

### Synthetic Example 8

### Synthesis of 1′-{[5-(trifluoromethyl)-2-furyl]methyl}spiro[1,3-dioxolo[4,5-b]furo[2,3-e]pyridine-5,3′-indol]-2′(1′H)-one

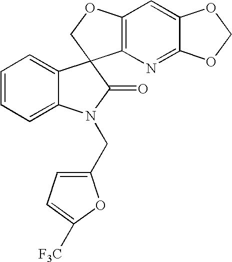

A mixture of 3-(6-hydroxy[1,3]dioxolo[4,5-b]pyridin-5-yl)-3-(hydroxymethyl)-1-{[5-(trifluoromethyl)-2-furyl]methyl}-1,3-dihydro-2H-indol-2-one (0.35 g, 0.78 mmol), triphenylphosphine (0.31 g, 1.2 mmol), diethyl azodicarboxylate (0.19 mL, 1.2 mmol) in anhydrous tetrahydrofuran (20.0 mL) was stirred at ambient temperature overnight and the organic solvent was evaporated in vacuo. The residue was subjected to column chromatography eluted with 20% ethyl acetate in hexanes to afford 1′-{[5-(trifluoromethyl)-2-furyl]methyl}spiro[1,3-dioxolo[4,5-b]furo[2,3-e]pyridine-5,3′-indol]-2′(1′H)-one (0.19 g, 58%): mp 175-177° C.; ¹H NMR (300 MHz, CDCl₃) δ 7.28 (ddd, J=7.6, 7.6, 1.2 Hz, 1H), 7.16 (dd, J=7.6, 1.2 Hz, 1H), 7.06 (ddd, J=7.6, 7.6, 0.9 Hz, 1H), 6.94 (d, J=8.1 Hz, 1H), 6.73 (s, 1H), 6.72-6.68 (m, 1H), 6.42 (d, J=3.2 Hz, 1H), 5.98 (s, 2H), 5.04 (d, J=9.3 Hz, 1H), 5.04-4.88 (m, 2H), 4.76 (d, J=9.3 Hz, 1H); ¹³C NMR (75 MHz, CDCl₃) δ 176.3, 153.3, 151.9, 151.0, 141.8, 141.2, 135.2, 131.0, 129.2, 123.8, 123.7, 112.7 (m), 109.2, 101.0, 80.1, 58.0, 37.2; MS (ES+) m/z 431.3 (M+1).

### Synthetic Example 9

### Synthesis of 5-methoxy-1′-{[5-(trifluoromethyl)-2-furyl]methyl}spiro[furo[3,2-b]pyridine-3,3′-indol]-2′(1′H)-one 4-oxide

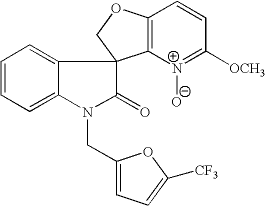

A mixture of 5-methoxy-1′-{[5-(trifluoromethyl)-2-furyl]methyl}spiro[furo[3,2-b]pyridine-3,3′-indol]-2′(1′H)-one (0.07 g, 0.17 mmol), m-chloroperoxybenzoic acid (0.18 g, 1.04 mmol) in dichloromethane (15 mL) was stirred at ambient temperature for one week under nitrogen. The reaction was quenched with saturated aqueous sodium bicarbonate (15 mL). The organic layer was separated and the aqueous layer was extracted with dichloromethane (2×20 mL). The combined organic layers was dried over anhydrous sodium sulfate and filtered. The filtrate was concentrated in vacuo to dryness. The residue was purified by preparative thin layer chromatography and developed with ethyl acetate:hexane (20%) to afford 5-methoxy-1′-{[5-(trifluoromethyl)-2-furyl]methyl}spiro[furo[3,2-b]pyridine-3,3′-indol]-2′(1′H)-one 4-oxide (0.02 g, 15%) as a solid: MS (ES+) m/z 433.2 (M+1), 455.2 (M+23).

### Synthetic Example 10

### Synthesis of 5′-fluoro-5-methoxy-1′-{[5-(trifluoromethyl)-2-furyl]methyl}spiro[furo[3,2-b]pyridine-3,3′-indol]-2′(1′H)-one

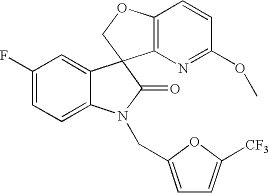

To a solution of 5-fluoro-3-(3-hydroxy-6-methoxypyridin-2-yl)-3-(hydroxymethyl)-1-{[5-(trifluoromethyl)-2-furyl]methyl}-1,3-dihydro-2H-indol-2-one (2.89 g, 6.40 mmol) in anhydrous tetrahydrofuran (100.0 mL) was added tributylphosphine (1.62 g, 1.9 mL, 8.00 mmol) followed by diethyl azodicarboxylate (1.39 g, 1.3 mL, 8.00 mmol) at 0° C. The reaction mixture was stirred at 0° C. for 1 h and quenched with saturated ammonium chloride solution (50 mL). After the organic solvent was removed in vacuo, the aqueous residue was extracted with ethyl acetate (3×50 mL). The combined organic layers was dried over sodium sulfate and filtered. The filtrate was concentrated in vacuo to dryness. The residue was purified by silica gel column chromatography eluted with ethyl acetate in hexanes (30%) to give 5′-fluoro-5-methoxy-1′-{[5-(trifluoromethyl)-2-furyl]methyl}spiro[furo[3,2-b]pyridine-3,3′-indol]-2′(1′H)-one (0.15 g, 5.4%) as a colourless solid: mp 158-160° C.; ¹H NMR (300 MHz, CDCl₃) δ 7.20 (d, J=8.7 Hz, 1H), 6.99 (ddd, J=9.0, 9.0, 2.7 Hz, 1H), 6.91-6.84 (m, 1H), 6.69 (d, J=2.4 Hz, 1H), 6.57 (d, J=8.7 Hz, 1H), 6.45 (d, J=3.0 Hz, 1H), 5.15 (d, J=16.5 Hz, 1H), 5.04 (d, J=9.3 Hz, 1H), 4.81 (d, J=16.5 Hz, 1H), 4.74 (d, J=9.3 Hz, 1H), 3.64 (s, 3H); ¹³C NMR (75 MHz, CDCl₃) δ 175.8, 160.3, 158.1, 151.8, 149.3, 144.7, 137.6, 132.7, 121.5, 115.6, 115.3, 112.6 (m), 112.2, 111.8, 111.0, 109.6, 108.9, 78.8, 59.1, 53.7, 37.3; MS (ES+) m/z 435.2 (M+1).

### Synthetic Example 11

### Synthesis of 4′-chloro-5-methoxy-1′-{[5-(trifluoromethyl)-2-furyl]methyl}spiro[furo[3,2-b]pyridine-3,3′-indol]-2′(1′H)-one

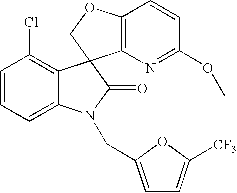

Following the procedure as described in SYNTHETIC EXAMPLE 10 and making non-critical variations to replace 5-fluoro-3-(3-hydroxy-6-methoxypyridin-2-yl)-3-(hydroxymethyl)-1-{[5-(trifluoromethyl)-2-furyl]methyl}-1,3-dihydro-2H-indol-2-one with 4-chloro-3-(3-hydroxy-6-methoxypyridin-2-yl)-3-(hydroxymethyl)-1-{[5-(trifluoromethyl)-2-furyl]methyl}-1,3-dihydro-2H-indol-2-one, 4′-chloro-5-methoxy-1′-{[5-(trifluoromethyl)-2-furyl]methyl}spiro[furo[3,2-b]pyridine-3,3′-indol]-2′(1′H)-one was obtained (78%) as a colourless solid: mp 106-108° C.; ¹H NMR (300 MHz, DMSO-d₆) δ 7.38 (d, J=8.8 Hz, 1H), 7.35 (dd, J=8.0, 8.0 Hz, 1H), 7.19 (dd, J=7.8, 7.8 Hz, 1H), 7.16 (dd, J=3.3, 1.1 Hz, 1H), 7.07 (d, J=8.0 Hz, 1H), 6.67-6.64 (m, 2H), 5.08 (ABq, J=16.6 Hz, 2H), 4.89 (ABq, J=10.1 Hz, 2H), 3.49 (s, 3H); ¹³C NMR (75 MHz, DMSO-d₆) δ 175.7, 159.8, 153.5, 150.7, 144.5, 143.3, 131.2, 130.2, 127.7, 124.0, 121.8, 114.5, 111.1, 109.9, 109.2, 108.8, 76.6, 59.0, 53.6, 37.4; MS (ES+) m/z 453.18 (M+1), 451.20 (M+1).

### Synthetic Example 12

### Synthesis of 4′-bromo-5-methoxyspiro[furo[3,2-b]pyridine-3,3′-indol]-2′(1′H)-one

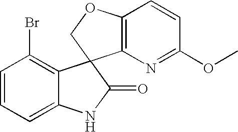

To a solution of 4-bromo-3-(3-hydroxy-6-methoxypyridin-2-yl)-1,3-bis(hydroxymethyl)-1,3-dihydro-2H-indol-2-one (3.48 g, 8.83 mmol) in anhydrous tetrahydrofuran (80.0 mL) was added tributylphosphine followed by a solution of di-tert-butyl azodicarboxylate (2.54 g, 11.0 mmol) in anhydrous tetrahydrofuran (10.0 mL) at 0° C. The reaction solution was stirred for 1 h followed by the addition of ammonium hydroxide (5.0 mL) and continued stirring for 0.5 h. The mixture was concentrated in vacuo to dryness. The residue was dissolved in ethyl acetate (100.0 mL) and washed with 10% aqueous hydrochloric acid solution (2×50.0 mL), saturated ammonium chloride (30.0 mL), brine (30.0 mL), dried over anhydrous sodium sulfate and filtered. The filtrate was concentrated in vacuo to dryness. The residue was purified by column chromatography eluting with ethyl acetate (100%) to afford 4′-bromo-5-methoxyspiro[furo[3,2-b]pyridine-3,3′-indol]-2′(1′H)-one (1.28 g, 42%) as a pale yellow solid: mp 265-268° C.; ¹H NMR (300 MHz, DMSO-d₆) δ 10.86 (s, 1H), 7.33 (d, J=8.8 Hz, 1H), 7.18 (dd, J=7.6, 7.6 Hz, 1H), 7.10 (d, J=8.2 Hz, 1H), 6.91 (d, J=7.5 Hz, 1H), 6.64 (d, J=8.8 Hz, 1H), 4.84 (ABq, J=9.9 Hz, 2H), 3.54 (s, 3H); ¹³C NMR (75 MHz, DMSO-d₆) δ 177.4, 159.7, 151.1, 144.9, 143.9, 131.2, 130.1, 126.0, 121.4, 119.1, 110.4, 109.8, 76.7, 60.3, 53.9; MS (ES+) m/z 349.2 (M+1), 347.2 (M+1).

### Synthetic Example 13

### Synthesis of 5-methoxyspiro[furo[3,2-b]pyridine-3,3′-indol]-2′(1′H)-one

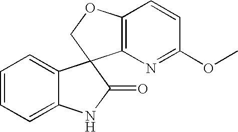

Following the procedure as described in SYNTHETIC EXAMPLE 5 and making non-critical variations to replace 4′-bromo-5-methoxy-1′-{[5-(trifluoromethyl)-2-furyl]methyl}spiro[furo[3,2-b]pyridine-3,3′-indol]-2′(1′H)-one with 4′-bromo-5-methoxyspiro[furo[3,2-b]pyridine-3,3′-indol]-2′(1′14)-one, 5-methoxyspiro[furo[3,2-b]pyridine-3,3′-indol]-2′(1′H)-one was obtained (69%) as a colourless solid: mp 199-200° C.; ¹H NMR (300 MHz, DMSO-d₆) δ 10.62 (s, 1H), 7.38 (d, J=8.8 Hz, 1H), 7.21 (dd, J=7.7, 7.7 Hz, 1H), 7.08 (d, J=7.2 Hz, 1H), 6.94 (d, J=7.5 Hz, 1H), 6.89 (d, J=8.0 Hz, 1H), 6.63 (d, J=8.8 Hz, 1H), 4.77 (ABq, J=9.5 Hz, 2H), 3.53 (s, 3H); ¹³C NMR (75 MHz, DMSO-d₆) δ 177.8, 159.9, 149.8, 146.5, 142.6, 132.1, 129.3, 124.3, 122.7, 121.8, 110.3, 110.2, 79.4, 59.0, 53.9; MS (ES+) m/z 269.3 (M+1), 241.3 (M−17).

### Synthetic Example 14

### Synthesis of 4′-bromo-1′-[(2-isopropyl-1,3-thiazol-5-yl)methyl]-5-methoxyspiro[furo[3,2-b]pyridine-3,3′-indol]-2′(1′H)-one

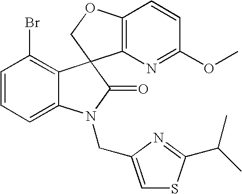

A mixture of 4′-bromo-5-methoxyspiro[furo[3,2-b]pyridine-3,3′-indol]-2′(1′H)-one (0.16 g, 0.46 mmol), 4-chloromethyl-2-isopropylthiazole (0.12 g, 0.69 mmol) and cesium carbonate (0.23 g, 0.69 mmol) in acetone (2.0 mL) was stirred at ambient temperature for 16 h. The solid was filtered and the residue was washed with ethyl acetate (5.0 mL). The filtrate was concentrated in vacuo to dryness. The residue was purified by column chromatography eluting with ethyl acetate in hexane (30%) to afford 4′-bromo-1′-[(2-isopropyl-1,3-thiazol-5-yl)methyl]-5-methoxyspiro[furo[3,2-b]pyridine-3,3′-indol]-2′(1′H)-one (0.02 g, 7.6%) as a colourless solid: mp 158-160° C.; ¹H NMR (300 MHz, DMSO-d₆) δ 7.37 (d, J=8.8 Hz, 1H), 7.26-7.24 (m, 2H), 7.21 (d, J=3.9 Hz, 1H), 7.10 (dd, J=7.2, 1.5 Hz, 1H), 6.67 (d, J=8.8 Hz, 1H), 5.10-4.93 (m, 3H), 4.82 (d, J=10.1 Hz, 1H), 3.55 (s, 3H); ¹³C NMR (75 MHz, DMSO-d₆) δ 178.4, 175.8, 159.8, 151.1, 150.3, 145.2, 143.5, 131.4, 129.3, 126.9, 121.7, 119.0, 114.3, 111.0, 109.4, 76.5, 59.9, 53.9, 41.0, 32.9, 23.2; MS (ES+) m/z 490.4 (M+1), 488.3 (M+1).

### Synthetic Example 15

### Synthesis of 1′-[(5-chloro-2-thienyl)methyl]-5-methoxyspiro[furo[3,2-b]pyridine-3,3′-indol]-2′(1′H)-one

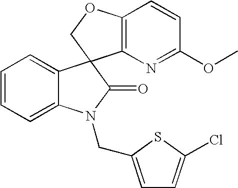

Following the procedure as described in EXAMPLE 14 and making non-critical variations to replace 4′-bromo-5-methoxyspiro[furo[3,2-b]pyridine-3,3′-indol]-2′(1′H)-one with 5-methoxyspiro[furo[3,2-b]pyridine-3,3′-indol]-2′(1′H)-one and 4-chloromethyl-2-isopropylthiazole with 5-chloromethyl-2-chlorothiphene, 1′-[(5-chloro-2-thienyl)methyl]-5-methoxyspiro[furo[3,2-b]pyridine-3,3′-indol]-2′(1′H)-one was obtained (78%) as a colourless solid: mp 130-132° C.; ¹H NMR (300 MHz, DMSO-d₆) δ 7.41 (d, J=8.8 Hz, 1H), 7.28 (dd, J=7.7, 7.7 Hz, 1H), 7.18 (d, J=4.3 Hz, 1H), 7.16 (d, J=4.5 Hz, 1H), 7.07 (d, J=3.8 Hz, 1H), 7.01 (d, J=7.6 Hz, 1H), 6.94 (d, J=3.8 Hz, 1H), 6.65 (d, J=8.8 Hz, 1H), 5.07 (ABq, J=16.1 Hz, 2H), 4.83 (ABq, J=9.7 Hz, 2H), 3.52 (s, 3H); ¹³C NMR (75 MHz, DMSO-d₆) δ 176.0, 159.9, 149.7, 146.1, 142.3, 138.4, 131.3, 129.4, 128.1, 127.1, 126.8, 124.257, 123.7, 122.1, 110.8, 109.9, 79.0, 58.5, 53.7, 38.9; MS (ES+) m/z 401.2 (M+1), 399.2 (M+1).

### Synthetic Example 16

### Synthesis of 1-[(2-isopropyl-1,3-thiazol-4-yl)methyl]-5-methoxyspiro[furo[3,2-b]pyridine-3,3′-indol]-2′(1′H)-one

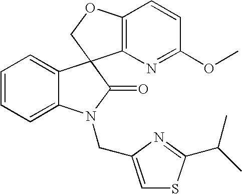

Following the procedure as described in EXAMPLE 14 and making non-critical variations to replace 4′-bromo-5-methoxyspiro[furo[3,2-b]pyridine-3,3′-indol]-2′(1′H)-one with 5-methoxyspiro[furo[3,2-b]pyridine-3,3′-indol]-2′(1′H)-one, 1′[(2-isopropyl-1,3-thiazol-4-yl)methyl]-5-methoxyspiro[furo[3,2-b]pyridine-3,3′-indol]-2′(1′H)-one was obtained (64%) as a colourless solid: mp 136-138° C.; ¹H NMR (300 MHz, DMSO-d₆) δ 7.42 (d, J=8.8 Hz, 1H), 7.29-7.23 (m, 2H), 7.19 (d, J=6.8 Hz, 1H), 7.06-6.99 (m, 2H), 6.65 (d, J=8.8 Hz, 1H), 5.01 (ABq, 2H), 4.86 (ABq, 2H), 3.54 (s, 3H), 3.27-3.18 (m, 1H), 1.28 (d, J=6.9 Hz, 6H); ¹³C NMR (75 MHz, DMSO-d₆) δ 178.3, 176.1, 159.9, 150.7, 149.7, 146.2, 142.9, 131.3, 129.4, 124.2, 123.6, 122.2, 114.2, 110.8, 109.9, 79.1, 58.6, 53.9, 40.7, 33.0, 23.2; MS (ES+) m/z 408.31 (M+1).

### Synthetic Example 17

### Synthesis of 5-methoxy-1′-(pyridin-2-ylmethyl)spiro[furo[3,2-b]pyridine-3,3′-indol]-2′(1′H)-one

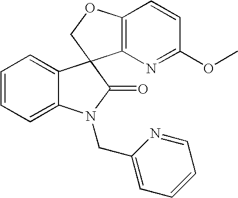

Following the procedure as described in EXAMPLE 14 and making non-critical variations to replace 4′-bromo-5-methoxyspiro[furo[3,2-b]pyridine-3,3′-indol]-2′(1′H)-one with 5-methoxyspiro[furo[3,2-b]pyridine-3,3′-indol]-2′(1′H)-one and 4-chloromethyl-2-isopropylthiazole with 2-bromomethylpyridine, 5-methoxy-1′-(pyridin-2-ylmethyl)spiro[furo[3,2-b]pyridine-3,3′-indol]-2′(1′H)-one was obtained (75%) as a colourless solid: mp 168-171° C.; ¹H NMR (300 MHz, DMSO-d₆) δ 8.57 (d, J=4.8 Hz, 1H), 7.72 (dt, J=7.7, 1.8 Hz, 1H), 7.46 (d, J=8.8 Hz, 1H), 7.37 (d, J=7.8 Hz, 1H), 7.33-7.23 (m, 3H), 7.05 (dt, J=7.5, 0.8 Hz, 1H), 6.94 (d, J=7.7 Hz, 1H), 6.70 (d, J=8.8 Hz, 1H), 5.19 (d, J=16.7 Hz, 1H), 4.97-4.86 (m, 3H), 3.62 (s, 3H); ¹³C NMR (75 MHz, DMSO-d₆) δ 175.8, 159.4, 155.4, 149.4, 149.1, 145.7, 142.5, 137.0, 130.8, 128.9, 123.7, 123.0, 122.6, 121.7, 120.3, 110.2, 109.3, 78.5, 58.1, 53.3, 44.9; MS (ES+) m/z 360.4 (M+1).

### Synthetic Example 18

### Synthesis of N-(2-fluorophenyl)-2-(5-methoxy-2′-oxospiro[furo[3,2-b]pyridine-3,3′-indol]-1′(2′H)-yl)acetamide

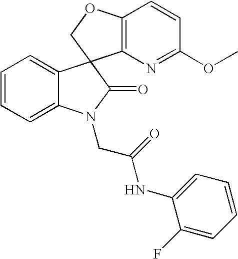

Following the procedure as described in EXAMPLE 14 and making non-critical variations to replace 4′-bromo-5-methoxyspiro[furo[3,2-b]pyridine-3,3′-indol]-2′(1′H)-one with 5-methoxyspiro[furo[3,2-b]pyridine-3,3′-indol]-2′(1′H)-one and 4-chloromethyl-2-isopropylthiazole with 2-chloro-N-(2-fluorophenyl)acetamide, N-(2-fluorophenyl)-2-(5-methoxy-2′-oxospiro[furo[3,2-b]pyridine-3,3′-indol]-1′(2′H)-yl)acetamide was obtained (56%) as a colourless solid: mp>170° C.; ¹H NMR (300 MHz, DMSO-d₆) δ 10.17 (s, 1H), 7.93-7.87 (m, 1H), 7.45 (d, J=8.8 Hz, 1H), 7.37-7.04 (m, 7H), 6.70 (d, J=8.8 Hz, 1H), 4.85 (d, J=20.6, 9.5 Hz, 2H), 4.72 (s, 2H), 3.58 (s, 3H); ¹³C NMR (75 MHz, DMSO-d₆) δ 176.1, 165.4, 159.4, 153.4 (d, J_{C—F}=245 Hz), 149.4, 145.4, 143.1, 130.3, 128.9, 125.8, 125.6, 125.5 (d, J_{C—F}=7 Hz), 124.4 (d, J_{C—F}=3 Hz), 123.7, 123.0, 121.5, 115.5 (d, ²J_{C—F}=19.3 Hz), 109.9, 109.2, 79.0, 58.0, 53.5, 43.0; MS (ES+) m/z 420.2 (M+1).

### Synthetic Example 19

### Synthesis of 1′-[(5-chloro-1-methyl-1H-imidazol-2-yl)methyl]-5-methoxyspiro[furo[3,2-b]pyridine-3,3′-indol]-2′(1′H)-one

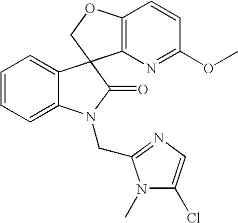

Following the procedure as described in EXAMPLE 14 and making non-critical variations to replace 4′-bromo-5-methoxyspiro[furo[3,2-b]pyridine-3,3′-indol]-2′(1′14)-one with 5-methoxyspiro[furo[3,2-b]pyridine-3,3′-indol]-2′(1′H)-one and 4-chloromethyl-2-isopropylthiazole with 5-chloro-(2-chloromethyl)-1-methyl-1H-imidazole, 1′-[(5-chloro-1-methyl-1H-imidazol-2-yl)methyl]-5-methoxyspiro[furo[3,2-b]pyridine-3,3′-indol]-2′(1′H)-one was obtained (76%) as a colourless solid: mp 169-171° C.; ¹H NMR (300 MHz, DMSO-d₆) δ 7.46 (d, J=8.8 Hz, 1H), 7.33-7.18 (m, 3H), 7.04 (ddd, J=7.3 Hz, J=7.3 Hz, J=1.1 Hz, 1H), 6.99 (s, 1H), 6.69 (d, J=8.8 Hz, 1H), 5.28 (d, J=15.8 Hz, 1H), 4.91 (d, J=9.7 Hz, 1H), 4.90 (d, J=15.8 Hz, 1H), 4.80 (d, J=9.7 Hz, 1H), 3.55 (s, 3H), 3.54 (s, 3H); ¹³C NMR (75 MHz, DMSO-d₆) δ 175.3, 159.4, 149.3, 145.4, 142.3, 142.1, 130.7, 128.7, 123.8, 123.5, 123.1, 121.6, 117.6, 110.2, 110.0, 78.9, 58.1, 53.3, 37.4, 30.4; MS (ES+) m/z 397.2 (M+1), 399.2 (M+1).

### Synthetic Example 20

### Synthesis of tert-butyl 4-[(4′-bromo-5-methoxy-2′-oxospiro[furo[3,2-b]pyridine-3,3′-indol]-1′(2′H)-yl)methyl]piperidine-1-carboxylate

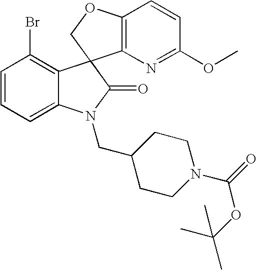

Following the procedure as described in EXAMPLE 14 and making non-critical variations to replace 4-chloromethyl-2-isopropylthiazole with tert-butyl 4-({[(4-methylphenyl)sulfonyl]oxy}methyl)piperidine-1-carboxylate, tert-butyl 4-[(4′-bromo-5-methoxy-2′-oxospiro[furo[3,2-b]pyridine-3,3′-indol]-1′(2′H)-yl)methyl]piperidine-1-carboxylate was obtained (1.80 g, quantitative yield) as a gummy solid: R_{f}=0.45 (ethyl acetate in hexane, 50%).

### Synthetic Example 21

### Synthesis of 5-methoxy-1′-(piperidin-4-ylmethyl)spiro[furo[3,2-b]pyridine-3,3′-indol]-2′(1′H)-one

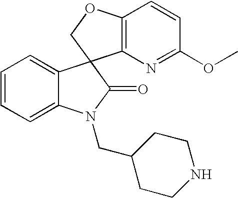

A mixture of tert-butyl 4-[(4′-bromo-5-methoxy-2′-oxospiro[furo[3,2-b]pyridine-3,3′-indol]-1′(2′H)-yl)methyl]piperidine-1-carboxylate (2.10 g, 3.85 mmol), tetrakis(triphenylphosphine)palladium (0) (1.10 g, 0.96 mmol), formic acid (2.31 g, 50.1 mmol) and triethylamine (5.07 g, 50.1 mmol) in anhydrous dioxane (50.0 mL) was heated at reflux for 16 h. The reaction solution was filter through a pad of celite. The filtrate was concentrated in vacuo to dryness. The residue was dissolved in ethyl acetate (100.0 mL) and the precipitate was filtered. The filtrate was concentrated in vacuo to dryness. The residue was re-dissolved in anhydrous dichloromethane (20.0 mL) followed by the addition of trifluoroacetic acid (10.0 mL) at 0° C. The reaction mixture was stirred for 1.5 h and the mixture was concentrated in vacuo to dryness. The residue was purified by column chromatography eluting with dichloromethane in methanol (10%) and ammonium hydroxide (0.10%) to afford 5-methoxy-1′-(piperidin-4-ylmethyl)spiro[furo[3,2-b]pyridine-3,3′-indol]-2′(1′H)-one (0.40 g, 28%) as a fluffy solid: MS (ES+) m/z 388.1 (M+23), 366.1 (M+1).

### Synthetic Example 22

### Synthesis of 5-methoxy-1′-[(1-methylpiperidin-4-yl)methyl]spiro[furo[3,2-b]pyridine-3,3′-indol]-2′(1′H)-one

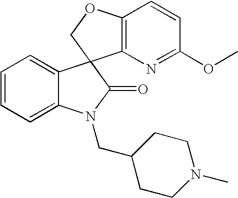

In a sealed tube, a mixture of 5-methoxy-1′-(piperidin-4-ylmethyl)spiro[furo[3,2-b]pyridine-3,3′-indol]-2′(1′H)-one (0.30 g, 0.82 mmol), formaldehyde solution (0.50 g, 16.4 mmol, 37 wt. % in water) and formic acid (1.52 g, 32.8 mmol) in water (2.0 mL) was heat at 100° C. for 7 h. The reaction mixture was basified with ammonium hydroxide and extracted with dichloromethane (3×25 mL), dried over anhydrous sodium sulfate and filtered. The filtrate was concentrated in vacuo to dryness. The residue was purified by column chromatography eluting with dichloromethane in methanol (5.0%) and ammonium hydroxide (0.10%) to afford 5-methoxy-1-[(1-methylpiperidin-4-yl)methyl]spiro[furo[3,2-b]pyridine-3,3′-indol]-2′(1′H)-one (0.20 g, 42%) as a colourless solid: mp 58-62° C.; ¹H NMR (300 MHz, DMSO-d₆) δ 7.43 (d, J=8.8 Hz, 1H), 7.33 (dd, J=7.7 Hz, J=7.7 Hz, 1H), 7.18 (d, J=7.8 Hz, 2H), 7.03 (dd, J=7.5 Hz, J=7.5 Hz, 1H), 6.66 (d, J=8.8 Hz, 1H), 4.83 (q, J=9.7 Hz, 2H), 3.73 (dd, J=13.8 Hz, J=8.0 Hz, 1H), 3.56-3.50 (m, 1H), 3.52 (s, 3H), 2.73 (t, J=11.4 Hz, 2H), 2.12 (s, 3H), 1.83-1.68 (m, 5H), 1.35-1.16 (m, 2H); ¹³C NMR (75 MHz, DMSO-d₆) δ 175.7, 159.3, 149.0, 145.8, 143.0, 130.9, 128.8, 123.4, 122.6, 121.5, 110.2, 109.2, 78.5, 58.0, 54.8, 54.8, 53.0, 46.0, 44.8, 33.5, 29.3, 29.1; MS (ES+) m/z 380.1 (M+1).

### Synthetic Example 23

### Synthesis of 5-methoxy-1′-{[5-(trifluoromethyl)furan-2-yl]methyl}spiro[furo[2,3-c]pyridine-3,3′-indol]-2′(1′H)-one

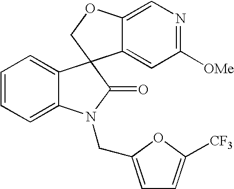

To a cooled (−78° C.) solution of 3-(5-hydroxy-2-methoxypyridin-4-yl)-3-(hydroxymethyl)-1-{[5-(trifluoromethyl)furan-2-yl]methyl}-1,3-dihydro-2H-indol-2-one (0.23 g, 0.53 mmol) in anhydrous tetrahydrofuran (5 mL) was added triphenylphosphine (0.16 g, 0.63 mmol) and N,N-diethyl azodicarboxylate (0.12 mL, 0.74 mmol). The reaction mixture was allowed to warm to ambient temperature. After stirring for 3 h, the reaction mixture was concentrated in vacuo and the residue was purified by column chromatography, eluting with a 10 to 50% gradient of ethyl acetate in hexanes to afford 5-methoxy-1′-{[5-(trifluoromethyl)furan-2-yl]methyl}spiro[furo[2,3-c]pyridine-3,3′-indol]-2′(1′H)-one (0.057 g, 26%) as a colorless gum: ¹H NMR (300 MHz, CDCl₃) δ 7.86 (s, 1H), 7.38-7.32 (m, 1H), 7.18-6.99 (m, 3H), 6.78-6.74 (m, 1H), 6.43-6.38 (m, 1H), 6.13 (s, 1H), 5.11-4.86 (m, 3H), 4.70 (d, J=9.0 Hz, 1H), 3.83 (s, 3H); ¹³C NMR (75 MHz, CDCl₃) δ 175.6, 159.2, 153.0, 151.8, 142.3, 141.5, 130.8, 129.6, 127.1, 124.2, 124.1, 120.6, 117.1, 112.8, 109.5, 109.3, 105.5, 80.0, 57.8, 54.9, 37.1; MS (ES+) m/z 417.2 (M+1).

### Synthetic Example 24

### Synthesis of 1′-(diphenylmethyl)-5-methoxyspiro[furo[2,3-c]pyridine-3,3′-indol]-2′(1′H)-one

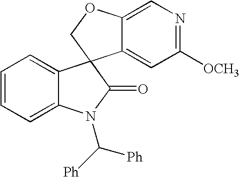

A solution of 1-(diphenylmethyl)-3-(5-hydroxy-2-methoxypyridin-4-yl)-1,3-dihydro-2H-indol-2-one (3.33 g, 7.88 mmol) in anhydrous tetrahydrofuran (50 mL) was degassed with dry argon for 1 h. Cesium carbonate (9.00 g, 27.6 mmol) was added in one portion, followed by chloroiodomethane (1.7 mL, 24 mmol). The reaction mixture was stirred at ambient temperature for 16 h and was filtered through a pad of diatomaceous earth. The pad was washed with ethyl acetate (50 mL) and the filtrate was concentrated in vacuo. The residue was purified by column chromatography, eluting with a 5 to 50% gradient of ethyl acetate in hexanes, to afford (diphenylmethyl)-5-methoxyspiro[furo[2,3-c]pyridine-3,3′-indol]-2′(1′H)-one (1.37 g, 40%) as a colorless solid: mp 177-178° C. (hexanes/ether); ¹H NMR (300 MHz, CDCl₃) δ 7.86 (s, 1H), 7.43-7.27 (m, 10H), 7.15-6.96 (m, 4H), 6.52 (d, J=7.6 Hz, 1H), 6.13 (s, 1H), 5.00 (d, J=9.0 Hz, 1H), 4.75 (d, J=9.0 Hz, 1H), 3.81 (s, 3H); ¹³C NMR (75 MHz, CDCl₃) δ 176.2, 159.1, 153.1, 142.9, 141.9, 137.5, 137.3, 131.2, 129.0, 128.9, 128.5, 128.4, 128.2 (2C), 127.1, 123.9, 123.5, 112.6, 105.6, 80.3, 59.0, 57.7, 54.0; MS (ES+) m/z 435.0 (M+1).

### Synthetic Example 25

### Synthesis of 5-methoxyspiro[furo[2,3-c]pyridine-3,3′-indol]-2′(1′H)-one

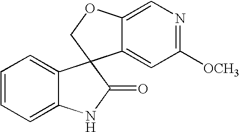

To a solution of 1′-(diphenylmethyl)-5-methoxyspiro[furo[2,3-c]pyridine-3,3′-indol]-2′(1′H)-one (0.91 g, 2.1 mmol) in trifluoroacetic acid (20 mL) was added triethylsilane (1.7 mL, 10 mmol) and the reaction mixture was heated at reflux for 24 h. The reaction mixture was allowed to cool to ambient temperature and was concentrated in vacuo. The residue was partitioned between ethyl acetate (25 mL) and sodium hydroxide (1 M, 25 mL). The phases were separated and the aqueous phase was extracted with ethyl acetate (3×25 mL). The combined organic extracts was washed with water (2×25 mL) and brine (25 mL), dried over anhydrous sodium sulfate, filtered and concentrated in vacuo. The crude product was triturated with diethyl ether (20 mL). The solid was collected by vacuum filtration and was washed with ice-cold diethyl ether (20 mL), air-dried and dried under high vacuum to afford 5-methoxyspiro[furo[2,3-c]pyridine-3,3′-indol]-2′(1′H)-one (0.281 g, 50%) as an off-white solid: mp 231-232° C. (ethyl acetate); ¹H NMR (300 MHz, DMSO-d₆) δ10.76 (br s, 1H), 7.87 (s, 1H), 7.32-7.25 (m, 1H), 7.15 (d, J=7.3 Hz, 1H), 7.02-6.91 (m, 2H), 6.24 (s, 1H), 4.85 (d, J=9.3 Hz, 1H), 4.72 (d, J=9.3 Hz, 1H), 3.71 (s, 3H); ¹³C NMR (75 MHz, DMSO-d₆) δ176.9, 158.3, 152.9, 143.3, 132.0, 131.1, 129.2, 126.2, 123.9, 122.5, 110.0, 105.0, 79.6, 57.6, 53.5; MS (ES+) m/z 268.7 (M+1).

### Synthetic Example 26

### Synthesis of 5-methoxy-1′-(tetrahydro-2H-pyran-4-ylmethyl)spiro[furo[2,3-c]pyridine-3,3′-indol]-2′(1′H)-one

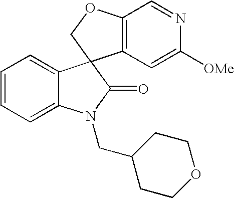

A heterogeneous mixture of 5-methoxyspiro[furo[2,3-c]pyridine-3,3′-indol]-2′(1′H)-one (0.134 g, 0.50 mmol), cesium carbonate (0.488 g, 1.50 mmol), 4-(bromomethyl)tetrahydropyran (0.447 g, 2.50 mmol) and 2-butanone (10 mL) was heated at reflux for 1.5 h. The reaction mixture was allowed to cool to ambient temperature and was filtered. The filtrate was concentrated in vacuo. The crude product was purified by column chromatography, eluting with a 10 to 50% gradient of ethyl acetate in hexanes to afford 5-methoxy-1′-(tetrahydro-2H-pyran-4-ylmethyl)spiro[furo[2,3-c]pyridine-3,3′-indol]-2′(1′H)-one (0.162 g, 89%) as a colorless solid: mp 84-85° C. (hexanes/ethyl acetate); ¹H NMR (300 MHz, CDCl₃) δ7.85 (s, 1H), 7.37-7.30 (m, 1H), 7.17-7.03 (m, 2H), 6.93 (d, J=7.9 Hz, 1H), 6.12 (s, 1H), 4.93 (d, J=9.0 Hz, 1H), 4.68 (d, J=9.0 Hz, 1H), 4.04-3.95 (m, 2H), 3.82 (s, 3H), 3.79-3.69 (m, 1H), 3.62-3.52 (m, 1H), 3.42-3.31 (m, 2H), 2.16-2.05 (m, 1H), 1.65-1.41 (m, 4H); ¹³C NMR (75 MHz, CDCl₃) δ176.1, 159.0, 142.8, 142.5, 131.0, 129.4, 126.9, 124.0, 123.6, 109.0, 105.4, 80.2, 67.4, 57.8, 53.8, 46.2, 33.9, 30.8, 30.7; MS (ES+) m/z 367.1 (M+1).

### Synthetic Example 27

### Synthesis of 5-methoxy-1′-[(2S)-tetrahydrofuran-2-ylmethyl]spiro[furo[2,3-c]pyridine-3,3′-indol]-2′(1′H)-one

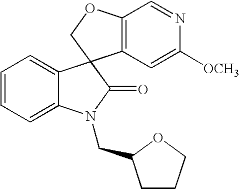

Following the procedure as described in SYNTHETIC EXAMPLE 26 and making non-critical variations to replace 4-(bromomethyl)tetrahydropyran with (2S)-tetrahydrofuran-2-ylmethyl 4-methylbenzenesulfonate, 5-methoxy-1′-[(2S)-tetrahydrofuran-2-ylmethyl]spiro[furo[2,3-c]pyridine-3,3′-indol]-2′(1′H)-one was obtained (27%) as a colorless solid: mp>250° C. (dichloromethane); ¹H NMR (300 MHz, CDCl₃) δ7.85 (s, 1H), 7.32 (t, J=7.5 Hz, 1H), 7.14-7.02 (m, 3H), 6.19 (d, J=3.9 Hz, 1H), 4.94 (d, J=9.0 Hz, 1H), 4.69 (d, J=9.0 Hz, 1H), 4.27 (m, 1H), 3.99-3.84 (m, 2H), 3.82 (s, 3H), 3.79-3.68 (m, 2H), 2.11-2.00 (m, 1H), 1.97-1.85 (m, 2H), 1.76-1.67 (m, 1H); ¹³C NMR (75 MHz, CDCl₃) δ 176.5, 176.3, 159.1, 153.1, 153.0, 143.0, 142.9, 142.8, 131.1, 130.9, 129.4, 129.3, 126.9, 123.7, 123.7, 123.6, 110.1, 109.9, 105.6, 105.5, 80.2, 76.9, 68.4, 68.3, 57.9, 57.8, 53.9, 44.9, 44.8, 29.4, 29.1, 25.9, 25.7; MS (ES+) m/z 353.1 (M+1).

### Synthetic Example 28

### Synthesis of 5-methoxy-1′-[(2R)-tetrahydrofuran-2-ylmethyl]spiro[furo[2,3-c]pyridine-3,3′-indol]-2′(1′H)-one

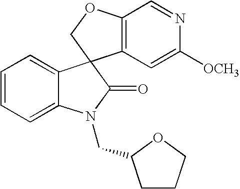

Following the procedure as described in SYNTHETIC EXAMPLE 26 and making non-critical variations to replace 4-(bromomethyl)tetrahydropyran with (2R)-tetrahydrofuran-2-ylmethyl 4-methylbenzenesulfonate, 5-methoxy-1-[(2R)-tetrahydrofuran-2-ylmethyl]spiro[furo[2,3-c]pyridine-3,3′-indol]-2′(1′H)-one was obtained (50%) as a colorless solid: mp>250° C. (hexanes/ethyl acetate); ¹H NMR (300 MHz, CDCl₃) δ7.84 (s, 1H), 7.35-7.29 (m, 1H), 7.14-7.00 (m, 4H), 6.19 (d, J=3.9 Hz, 1H), 4.94 (d, J=3.9 Hz, 1H), 4.69 (d, J=3.9 Hz, 1H), 6.87 (s, 1H), 6.36 (s, 1H), 4.81 (d, J=9.0 Hz, 1H), 4.70 (d, J=9.0 Hz, 1H), 4.33-4.21 (m, 1H), 3.99-3.69 (m, 8H), 2.11-1.85 (m, 4H), 1.76-1.67 (m, 1H); ¹³C NMR (75 MHz, CDCl₃) δ159.0, 153.0, 142.9, 131.0, 129.3, 129.2, 126.8, 123.6, 123.5, 123.4, 110.0, 109.8, 105.5, 105.4, 80.1, 68.3, 68.2, 57.8, 57.7, 53.8, 44.8, 44.7, 29.3, 29.0, 25.7, 25.6; MS (ES+) m/z 353.1 (M+1).

### Biological Assays

Various techniques are known in the art for testing the activity of compounds of the invention. In order that the invention described herein may be more fully understood, the following biological assays are set forth. It should be understood that these examples are for illustrative purposes only and are not to be construed as limiting this invention in any manner.

### Biological Example 1

### Guanidine Influx Assay (In Vitro Assay)

This example describes an in vitro assay for testing and profiling test agents against human or rat sodium channels stably expressed in cells of either an endogenous or recombinant origin. The assay is also useful for determining the IC-50 of a sodium channel blocking compound. The assay is based on the guanidine flux assay described by Reddy, N. L., et al., J Med Chem (1998), 41(17):3298-302.

The guanidine influx assay is a radiotracer flux assay used to determine ion flux activity of sodium channels in a high-throughput microplate-based format. The assay uses ¹⁴C-guanidine hydrochloride in combination with various known sodium channel modulators, to assay the potency of test agents. Potency is determined by an IC-50 calculation. Selectivity is determined by comparing potency of the compound for the channel of interest to its potency against other sodium channels (also called ‘selectivity profiling’).

Each of the test agents is assayed against cells that express the channels of interest. Voltage gated sodium channels are either TTX sensitive or insensitive. This property is useful when evaluating the activities of a channel of interest when it resides in a mixed population with other sodium channels. The following Table 1 summarizes cell lines useful in screening for a certain channel activity in the presence or absence of TTX.

TABLE 1

| CELL LINE | mRNA Expression | Functional Characterization |
|---|---|---|
| CHO-K1 (Chinese | Naᵥ1.4 expression has been | The 18-20-fold increase in [¹⁴C] |
| Hamster Ovary; | shown by RT-PCR | Guanidine influx was completely |
| recommended | No other Na_{V} expression has | blocked using TTX. (Na_{V}1.4 is a |
| host cell line) | been detected | TTX sensitive channel) |
| ATTC accession | | |
| number CCL-61 | | |
| L6 (rat myoblast | Expression of Nav1.4 and 1.5 | The 10-15 fold increase in [¹⁴C] |
| cell) ATTC | | Guanidine influx was only |
| Number CRL-1458 | | partially blocked by TTX (Naᵥ1.5 |
| | | is TTX resistant |
| SH-SY5Y (Human | Published Expression of | The 10-16-fold increase in [¹⁴C] |
| neuroblastoma) | Na_{V}1.9 and Na_{V}1.7 (Blum et | Guanidine influx above |
| ATTC Number | al) | background. |
| CRL-2266 | | was partially blocked by TTX |
| | | (Na_{V}1.9 is TTX resistant |
| SK-N-BE2C (a | Expression of NaV1.8 | Stimulation of BE2C cells with |
| human | | pyrethroids results in a 6 fold |
| neuroblastoma cell | | increase in [¹⁴C] Guanidine influx |
| line ATCC Number | | above background. |
| CRL-2268) | | TTX partially blocked influx |
| | | (NaV1.8 is TTX resistant) |
| PC12 (rat | Expression of Naᵥ1.2 | The 8-12-fold increase in [¹⁴C] |
| pheochromocytoma) | expression | Guanidine influx was completely |
| ATTC Number | | blocked using TTX. (Naᵥ1.2 is a |
| CRL-1721 | | TTX sensitive channel) |

It is also possible to employ recombinant cells expressing these sodium channels. Cloning and propagation of recombinant cells are known to those skilled in the art (see, for example, Klugbauer, N, et al., EMBO J. (1995), 14(6):1084-90; and Lossin, C., et al., Neuron (2002), 34, pp. 877-884).

Cells expressing the channel of interest are grown according to the supplier or in the case of a recombinant cell in the presence of selective growth media such as G418 (Gibco/Invitrogen). The cells are disassociated from the culture dishes with an enzymatic solution (1×) Trypsin/EDTA (Gibco/Invitrogen) and analyzed for density and viability using haemocytometer (Neubauer). Disassociated cells are washed and resuspended in their culture media then plated into Scintiplates (Beckman Coulter Inc.) (approximately 100,000 cells/well) and incubated at 37° C./5% CO₂ for 20-24 hours. After an extensive wash with Low sodium HEPES-buffered saline solution (LNHBSS) (150 mM Choline Chloride, 20 nM HEPES (Sigma), 1 mM Calcium Chloride, 5 mM Potassium Chloride, 1 mM Magnesium Chloride, 10 mM Glucose) agents diluted with LNHBSS are added to each well. (Varying concentrations of test agent may be used). The activation/radiolabel mixture contains aconitine (Sigma), and ¹⁴C-guanidine hydrochloride (ARC).

After loading the cells with test agent and activation/radiolabel mixture, the Scintiplates are incubated at ambient temperature. Following the incubation, the Scintplates are extensively washed with LNHBSS supplemented with guanidine (Sigma). The Scintiplates are dried and then counted using a Wallac MicroBeta TriLux (Perkin-Elmer Life Sciences). The ability of the test agent to block sodium channel activity is determined by comparing the amount of ¹⁴C-guanidine present inside the cells expressing the different sodium channels. Based on this data, a variety of calculations, as set out elsewhere in this specification, may be used to determine whether a test agent is selective for a particular sodium channel.

IC-50 value of a test agent for a specific sodium channel may be determined using the above general method. IC-50 may be determined using a 3, 8, 10, 12 or 16 point curve in duplicate or triplicate with a starting concentration of 1, 5 or 10 μM diluted serially with a final concentration reaching the sub-nanomolar, nanomolar and low micromolar ranges. Typically the mid-point concentration of test agent is set at 1 μM, and sequential concentrations of half dilutions greater or smaller are applied (e.g. 0.5 μM; 5 μM and 0.25 μM; 10 μM and 0.125 μM; 20 μM etc.). The IC-50 curve is calculated using the 4 Parameter Logistic Model or Sigmoidal Dose-Response Model formula (fit=(A+((B−A)/(1+((C/×)̂ D)))).

The fold selectivity, factor of selectivity or multiple of selectivity, is calculated by dividing the IC-50 value of the test sodium channel by the reference sodium channel, for example, Naᵥ1.5.
Representative compounds of the invention, when tested in the above assay using a known cell line that expresses a sodium channel, demonstrated an IC₅₀ (nM) activity level as set forth below in Table 2 wherein “A” refers to an IC₅₀ activity level of from 1 nM to 10 nM, “B” refers to an IC₅₀ activity level from 10 nM to 100 nM, “C” refers to an IC₅₀ activity level from 100 nM to 1000 nM, and “D” refers to an IC₅₀ activity level equal to or greater than 1000 nM. The Synthetic Example numbers provided in Table 2 correspond to the Synthetic Examples herein:

TABLE 2

| Synthetic | | IC₅₀ Activity |
|---|---|---|
| Example | Compound Name | Level |
| 1 | 1′-pentylspiro[furo[3,2-c]pyridine-3,3′-indol]-2′(1′H)-one | C |
| 2 | 1′-pentylspiro[furo[3,2-c]pyridine-3,3′indol]-2′(1′H)-one | D |
| | 5-oxide | |
| 3 | 1′-pentylspiro[furo[3,2-c]pyridine-3,3′-indole]- | D |
| | 2′,4(1′H,5H)-dione | |
| 4 | 4′-bromo-5-methoxy-1′-{[5-(trifluoromethyl)-2- | B |
| | furyl]methyl}spiro[furo[3,2-b]pyridine-3,3′-indol]- | |
| | 2′(1′H)-one | |
| 5 | 5-methoxy-1′-{[5-(trifluoromethyl)-2- | C |
| | furyl]methyl}spiro[furo[3,2-b]pyridine-3,3′-indol]- | |
| | 2′(1′H)-one | |
| 7 | 1′-{[5-(trifluoromethyl)-2-furyl]methyl}spiro[furo[3,2- | C |
| | b]pyridine-3,3′-indole]-2′,5(1′H,4H)-dione | |
| 8 | 1′-{[5-(trifluoromethyl)-2-furyl]methyl}spiro[1,3- | A |
| | dioxolo[4,5-b]furo[2,3-e]pyridine-5,3′-indol]- | |
| | 2′(1′H)-one | |
| 10 | 5′-fluoro-5-methoxy-1′-{[5-(trifluoromethyl)-2- | D |
| | furyl]methyl}spiro[furo[3,2-b]pyridine-3,3′-indol]- | |
| | 2′(1′H)-one | |
| 11 | 4′-chloro-5-methoxy-1′-{[5-(trifluoromethyl)-2- | C |
| | furyl]methyl}spiro[furo[3,2-b]pyridine-3,3′-indol]- | |
| | 2′(1′H)-one | |
| 12 | 4′-bromo-5-methoxyspiro[furo[3,2-b]pyridine-3,3′-indol]- | C |
| | 2′(1′H)-one | |
| 13 | 5-methoxyspiro[furo[3,2-b]pyridine-3,3′-indol]-2′(1′H)-one | D |
| 14 | 4′-bromo-1′-[(2-isopropyl-1,3-thiazol-5-yl)methyl]-5- | B |
| | methoxyspiro[furo[3,2-b]pyridine-3,3′-indol]- | |
| | 2′(1′H)-one | |
| 15 | 1′-[(5-chloro-2-thienyl)methyl]-5-methoxyspiro[furo[3,2- | B |
| | b]pyridine-3,3′-indol]-2′(1′H)-one | |
| 16 | 1′-[(2-isopropyl-1,3-thiazol-4-yl)methyl]-5- | B |
| | methoxyspiro[furo[3,2-b]pyridine-3,3′-indol]- | |
| | 2′(1′H)-one | |
| 17 | 5-methoxy-1′-(pyridin-2-ylmethyl)spiro[furo[3,2- | C |
| | b]pyridine-3,3′-indol]-2′(1′H)-one | |
| 18 | N-(2-fluorophenyl)-2-(5-methoxy-2′-oxospiro[furo[3,2- | C |
| | b]pyridine-3,3′-indol]-1′(2′H)-yl)acetamide | |
| 19 | 1′-[(5-chloro-1-methyl-1H-imidazol-2-yl)methyl]-5- | C |
| | methoxyspiro[furo[3,2-b]pyridine-3,3′-indol]- | |
| | 2′(1′H)-one | |
| 22 | 5-methoxy-1′-[(1-methylpiperidin-4- | D |
| | yl)methyl]spiro[furo[3,2-b]pyridine-3,3′-indol]- | |
| | 2′(1′H)-one | |
| 23 | 5-methoxy-1′-{[5-(trifluoromethyl)furan-2- | A |
| | yl]methyl}spiro[furo[2,3-c]pyridine-3,3′-indol]- | |
| | 2′(1′H)-one | |
| 24 | 1′-(diphenylmethyl)-5-methoxyspiro[furo[2,3-c]pyridine- | D |
| | 3,3′-indol]-2′(1′H)-one | |
| 25 | 5-methoxyspiro[furo[2,3-c]pyridine-3,3′-indol]-2′(1′H)-one | C |
| 26 | 5-methoxy-1′-(tetrahydro-2H-pyran-4- | C |
| | ylmethyl)spiro[furo[2,3-c]pyridine-3,3′-indol]- | |
| | 2′(1′H)-one | |
| 27 | 5-methoxy-1′-[(2S)-tetrahydrofuran-2- | B |
| | ylmethyl]spiro[furo[2,3-c]pyridine-3,3′-indol]- | |
| | 2′(1′H)-one | |
| 28 | 5-methoxy-1′-[(2R)-tetrahydrofuran-2- | B |
| | ylmethyl]spiro[furo[2,3-c]pyridine-3,3′-indol]- | |
| | 2′(1′H)-one | |

### Biological Example 2

### Electrophysiological Assay (In Vitro Assay)

Cells expressing the channel of interest were cultured in DMEM growth media (Gibco) with 0.5 mg/mL G418, +/−1% PSG, and 10% heat-inactivated fetal bovine serum at 37° C. and 5% CO₂. For electrophysiological recordings, cells were plated on 10 mm dishes.

Whole cell recordings were examined by established methods of whole cell voltage clamp (Bean et al., op. cit.) using an Axopatch 200B amplifier and Clampex software (Axon Instruments, Union City, Calif.). All experiments were performed at ambient temperature. Electrodes were fire-polished to resistances of 2-4 Mohms Voltage errors and capacitance artifacts were minimized by series resistance compensation and capacitance compensation, respectively. Data were acquired at 40 kHz and filtered at 5 kHz. The external (bath) solution consisted of: NaCl (140 mM), KCl (5 mM), CaCl₂ (2 mM), MgCl₂ (1 mM), HEPES (10 mM) at pH 7.4. The internal (pipette) solution consisted of (in mM): NaCl (5), CaCl₂ (0.1), MgCl₂ (2), CsCl (10), CsF (120), HEPES (10), EGTA (10), at pH 7.2.

To estimate the steady-state affinity of compounds for the resting and inactivated state of the channel (Kᵣ and Kᵢ, respectively), 12.5 ms test pulses to depolarizing voltages from ±60 to +90 mV from a holding potential of −110 mV was used to construct current-voltage relationships (1-V curves). A voltage near the peak of the IV-curve (−30 to 0 mV) was used as the test pulse throughout the remainder of the experiment. Steady-state inactivation (availability) curves were then constructed by measuring the current activated during a 8.75 ms test pulse following 1 second conditioning pulses to potentials ranging from −110 to −10 mV. To monitor channels at steady-state, a single “diary” protocol with a holding potential of −110 mV was created to record the resting state current (10 ms test pulse), the current after fast inactivation (5 ms pre-pulse of −80 to −50 mV followed by a 10 ms test pulse), and the current during various holding potentials (35 ms ramp to test pulse levels). Compounds were applied during the “diary” protocol and the block was monitored at 15 s intervals.

After the compounds equilibrated, the voltage-dependence of the steady-state inactivation in the presence of the compound was determined. Compounds that block the resting state of the channel decreased the current elicited during test pulses from all holding potentials, whereas compounds that primarily blocked the inactivated state decreased the current elicited during test pulses at more depolarized potentials. The currents at the resting state (Iᵣₑₛₜ) and the currents during the inactivated state (I_{inactivated}) were used to calculate steady-state affinity of compounds. Based on the Michaelis-Menton model of inhibition, the Kᵣ and Kᵢ was calculated as the concentration of compound needed to cause 50% inhibition of the Iᵣₑₛₜ or the I_{inactivated}, respectively.

$$\% inhibition = \frac{{{V}_{max}^{*} [ Drug ]}^{h}}{{[ Drug ]}^{h} + {K}_{m}^{h}}$$

Vₘₐₓ is the rate of inhibition, h is the Hill coefficient (for interacting sites), Kₘ is Michaelis-Menten constant, and [Drug] is the concentration of the test compound. At 50% inhibition (½Vₘₐₓ) of the Iᵣₑₛₜ or I_{inactivated}, the drug concentration is numerically equal to Kₘ and approximates the Kᵣ and Kᵢ, respectively.

### Biological Example 3

### Analgesia Induced by Sodium Channel Blockers

### Heat Induced Tail Flick Latency Test

In this test, the analgesia effect produced by administering a compound of the invention is observed through heat-induced tail-flick in mice. The test includes a heat source consisting of a projector lamp with a light beam focused and directed to a point on the tail of a mouse being tested. The tail-flick latencies, which are assessed prior to drug treatment, and in response to a noxious heat stimulus, i.e., the response time from applying radiant heat on the dorsal surface of the tail to the occurrence of tail flick, are measured and recorded at 40, 80, 120, and 160 minutes.

For example, a study can be designed wherein in the first part of the study, a certain number of animals undergo assessment of baseline tail flick latency once a day over two consecutive days. These animals are then randomly assigned to one of the several different treatment groups (depending on how many compounds are tested) including a vehicle control, a morphine control, and compounds are administered intramuscularly at 30 mg/kg. Following dose administration, the animals are closely monitored for signs of toxicity including tremor or seizure, hyperactivity, shallow, rapid or depressed breathing and failure to groom. The optimal incubation time for each compound is determined via regression analysis. The analgesic activity of the test compounds is expressed as a percentage of the maximum possible effect (% MPE) and is calculated using the following formula:

$$\% M P E \frac{Postdrug latency - Predrug latency}{Cut \text{-} off time ( 10 s ) - Predrug latency} \times 100 \%$$

where:

Postdrug latency=the latency time for each individual animal taken before the tail is removed (flicked) from the heat source after receiving drug.

Predrug latency=the latency time for each individual animal taken before the tail is flicked from the heat source prior to receiving drug.

Cut-off time (10 s)=is the maximum exposure to the heat source.

### Acute Pain (Formalin Test)

The formalin test is used as an animal model of acute pain. In the formalin test, animals are briefly habituated to the plexiglass test chamber on the day prior to experimental day for 20 minutes. On the test day, animals are randomly injected with the test compounds. At 30 minutes after drug administration, 50 μL of 10% formalin is injected subcutaneously into the plantar surface of the left hind paw of the rats. Video data acquisition begins immediately after formalin administration, for duration of 90 minutes.

The images are captured using the Actimetrix Limelight software which stores files under the *.llii extension, and then converts it into the MPEG-4 coding. The videos are then analyzed using behaviour analysis software “The Observer 5.1”, (Version 5.0, Noldus Information Technology, Wageningen, The Netherlands). The video analysis is done by watching the animal behaviour and scoring each according to type, and defining the length of the behaviour (Dubuisson and Dennis, 1977). Scored behaviours include: (1) normal behaviour, (2) putting no weight on the paw, (3) raising the paw, (4) licking/biting or scratching the paw. Elevation, favoring, or excessive licking, biting and scratching of the injected paw indicate a pain response. Analgesic response or protection from compounds is indicated if both paws are resting on the floor with no obvious favoring, excessive licking, biting or scratching of the injected paw.

Analysis of the formalin test data is done according to two factors: (1) Percent Maximal Potential Inhibitory Effect (% MPIE) and (2) pain score. The % MPIEs was calculated by a series of steps, where the first is to sum the length of non-normal behaviours (behaviours 1, 2, 3) of each animal. A single value for the vehicle group is obtained by averaging all scores within the vehicle treatment group. The following calculation yields the MPIE value for each animal:

MPIE(%)=100−[(treatment sum/average vehicle value)×100%]

The pain score is calculated from a weighted scale as described above. The duration of the behaviour is multiplied by the weight (rating of the severity of the response), and divided by the total length of observation to determine a pain rating for each animal. The calculation is represented by the following formula:

Pain rating=[0(To)+1(T1)+2(T2)+3(T3)]/(To+T1+T2+T3).

### CFA Induced Chronic Inflammatory Pain

In this test, tactile allodynia can be assessed with calibrated von Frey filaments. Following a full week of acclimatization to the vivarium facility, 150 μL of the “Complete Freund's Adjuvant” (CFA) emulsion (CFA suspended in an oil/saline (1:1) emulsion at a concentration of 0.5 mg/mL) was injected subcutaneously into the plantar surface of the left hind paw of rats under light isoflurane anaesthesia. Animals were allowed to recover from the anaesthesia and the baseline thermal and mechanical nociceptive thresholds of all animals were assessed one week after the administration of CFA. All animals were habituated to the experimental equipment for 20 minutes on the day prior to the start of the experiment. The test and control articles were administrated to the animals, and the nociceptive thresholds measured at defined time points after drug administration to determine the analgesic responses to each of the six available treatments. The time points used were previously determined to show the highest analgesic effect for each test compound.

Thermal nociceptive thresholds of the animals were assessed using the Hargreaves test. Animals were placed in a Plexiglas enclosure set on top of an elevated glass platform with heating units. The glass platform was thermostatically controlled at a temperature of approximately 30° C. for all test trials. Animals were allowed to accommodate for 20 minutes following placement into the enclosure until all exploration behaviour ceases. The Model 226 Plantar/Tail Stimulator Analgesia Meter (IITC, Woodland Hills, Calif.) was used to apply a radiant heat beam from underneath the glass platform to the plantar surface of the hind paws. During all test trials, the idle intensity and active intensity of the heat source were set at 1 and 45 respectively, and a cut off time of 20 seconds is employed to prevent tissue damage.

The response thresholds of animals to tactile stimuli were measured using the Model 2290 Electrovonfrey anesthesiometer (IITC Life Science, Woodland Hills, Calif.) following the Hargreaves test. Animals were placed in an elevated Plexiglas enclosure set on a mire mesh surface. After 10 minutes of accommodation, pre-calibrated Von Frey hairs are applied perpendicularly to the plantar surface of both paws of the animals in an ascending order starting from the 0.1 g hair, with sufficient force to cause slight buckling of the hair against the paw. Testing continues until the hair with the lowest force to induce a rapid flicking of the paw was determined or when the cut off force of approximately 20 g was reached. This cut off force was used because it represent approximately 10% of the animals' body weight and it served to prevent raising of the entire limb due to the use of stiffer hairs, which would change the nature of the stimulus.

Representative compounds of the invention, when tested in the above assay demonstrated % activity in alleviating the pain in the range of 23% to 98% as compared to a control analgesic compound.

### Postoperative Models of Nociception

In this model, the hypealgesia caused by an intra-planar incision in the paw can be measured by applying increased tactile stimuli to the paw until the animal withdraws its paw from the applied stimuli. While animals are anaesthetized under 3.5% isofluorane, which is delivered via a nose cone, a 1 cm longitudinal incision was made using a number 10 scalpel blade in the plantar aspect of the left hind paw through the skin and fascia, starting 0.5 cm from the proximal edge of the heel and extending towards the toes. Following the incision, the skin is apposed using 2, 3-0 sterilized silk sutures. The injured site is covered with Polysporin and Betadine. Animals were returned to their home cage for overnight recovery.

The withdrawal thresholds of animals to tactile stimuli for both operated (ipsilateral) and unoperated (contralateral) paws can be measured using the Model 2290 Electrovonfrey anesthesiometer (IITC Life Science, Woodland Hills, Calif.). Animals are placed in an elevated Plexiglas enclosure set on a mire mesh surface. After at least 10 minutes of acclimatization, pre-calibrated Von Frey hairs are applied perpendicularly to the plantar surface of both paws of the animals in an ascending order starting from the 10 g hair, with sufficient force to cause slight buckling of the hair against the paw. Testing continued until the hair with the lowest force to induce a rapid flicking of the paw is determined or when the cut off force of approximately 20 g is reached. This cut off force is used because it represent approximately 10% of the animals' body weight and it serves to prevent raising of the entire limb due to the use of stiffer hairs, which would change the nature of the stimulus.

### Neuropathic Pain Model; Chronic Constriction Injury

Briefly, an approximately 3 cm incision was made through the skin and the fascia at the mid thigh level of the animals' left hind leg using a no. 10 scalpel blade. The left sciatic nerve was exposed via blunt dissection through the biceps femoris with care to minimize haemorrhagia. Four loose ligatures were tied along the sciatic nerve using 4-0 non-degradable sterilized silk sutures at intervals of 1 to 2 mm apart. The tension of the loose ligatures was tight enough to induce slight constriction of the sciatic nerve when viewed under a dissection microscope at a magnification of 4 fold. In the sham-operated animal, the left sciatic nerve was exposed without further manipulation. Antibacterial ointment was applied directly into the wound, and the muscle was closed using sterilized sutures. Betadine was applied onto the muscle and its surroundings, followed by skin closure with surgical clips.

The response thresholds of animals to tactile stimuli were measured using the Model 2290 Electrovonfrey anesthesiometer (IITC Life Science, Woodland Hills, Calif.). Animals were placed in an elevated Plexiglas enclosure set on a mire mesh surface. After 10 minutes of accommodation, pre-calibrated Von Frey hairs were applied perpendicularly to the plantar surface of both paws of the animals in an ascending order starting from the 0.1 g hair, with sufficient force to cause slight buckling of the hair against the paw. Testing continues until the hair with the lowest force to induce a rapid flicking of the paw is determined or when the cut off force of approximately 20 g is reached. This cut off force is used because it represents approximately 10% of the animals' body weight and it serves to prevent raising of the entire limb due to the use of stiffer hairs, which would change the nature of the stimulus. Compounds of the present invention were shown to be efficacious within a range of 30 mg/kg and 0.1 mg/Kg.

Thermal nociceptive thresholds of the animals were assessed using the Hargreaves test. Following the measurement of tactile thresholds, animals were placed in a Plexiglass enclosure set on top of an elevated glass platform with heating units. The glass platform is thermostatically controlled at a temperature of approximately 24 to 26° C. for all test trials. Animals were allowed to accommodate for 10 minutes following placement into the enclosure until all exploration behaviour ceases. The Model 226 Plantar/Tail Stimulator Analgesia Meter (IITC, Woodland Hills, Calif.) was used to apply a radiant heat beam from underneath the glass platform to the plantar surface of the hind paws. During all test trials, the idle intensity and active intensity of the heat source were set at 1 and 55 respectively, and a cut off time of 20 seconds was used to prevent tissue damage.

### Biological Example 4

### Aconitine Induced Arrhythmia Test

The antiarrhythmic activity of compounds of the invention is demonstrated by the following test. Arrhythmia is provoked by intravenous administration of aconitine (2.0 μg/Kg) dissolved in physiological saline solution. Test compounds of the invention are intravenously administered 5 minutes after the administration of aconitine. Evaluation of the anti-arrhythmic activity is conducted by measuring the time from the aconitine administration to the occurrence of extrasystole (ES) and the time from the aconitine administration to the occurrence of ventricular tachycardia (VT).

In rates under isoflurane anaesthesia (¼ to ⅓ of 2%), a tracheotomy is performed by first creating an incision in the neck area, then isolating the trachea and making a 2 mm incision to insert tracheal tube 2 cm into the trachea such that the opening of the tube is positioned just on top of the mouth. The tubing is secured with sutures and attached to a ventilator for the duration of the experiment.

Incisions (2.5 cm) are then made into the femoral areas and using a blunt dissection probe, the femoral vessels are isolated. Both femoral veins are cannulated, one for pentobarbital anaesthetic maintenance (0.02-0.05 mL) and one for the infusion and injection of drug and vehicle. The femoral artery is cannulated with the blood pressure gel catheter of the transmitter.

The ECG leads are attached to the thoracic muscle in the Lead II position (upper right/above heart—white lead and lower left/below heart—red lead). The leads are secured with sutures.

All surgical areas are covered with gauze moistened with 0.9% saline. Saline (1-1.5 mL of a 0.9% solution) is supplied to moisten the areas post-surgery. The animals' ECG and ventilation are allowed to equilibrate for at least 30 minutes.

The arrhythmia is induced with a 2 μg/Kg/min aconitine infusion for 5 minutes. During this time the ECG is recorded and continuously monitored. An intravenous bolus injection of a test compound of the invention (10, 30 or 100 μg/kg) resulted in a complete return to normal baseline ECG.

### Biological Example 5

### Ischemia Induced Arrhythmia Test

Rodent models of ventricular arrhythmias, in both acute cardioversion and prevention paradigms have been employed in testing potential therapeutics for both atrial and ventricular arrhythmias in humans. Cardiac ischemia leading to myocardial infarction is a common cause of morbidity and mortality. The ability of a compound to prevent ischemia-induced ventricular tachycardia and fibrillation is an accepted model for determining the efficacy of a compound in a clinical setting for both atrial and ventricular tachycardia and fibrillation.

Anaesthesia is first induced by pentobarbital (i.p.), and maintained by an i.v. bolus infusion. Male SD rats have their trachea cannulated for artificial ventilation with room air at a stroke volume of 10 mL/Kg, 60 strokes/minute. The right femoral artery and vein are cannulated with PE50 tubing for mean arterial blood pressure (MAP) recording and intravenous administration of compounds, respectively.

The chest is opened between the 4^{th} and 5^{th} ribs to create a 1.5 cm opening such that the heart is visible. Each rat is placed on a notched platform and metal restraints are hooked onto the rib cage opening the chest cavity. A suture needle is used to penetrate the ventricle just under the lifted atrium and exited the ventricle in a downward diagonal direction so that a >30% to <50% occlusion zone (OZ) would be obtained. The exit position is ˜0.5 cm below where the aorta connects to the left ventricle. The suture is tightened such that a loose loop (occluder) is formed around a branch of the artery. The chest is then closed with the end of the occluder accessible outside of the chest.

Electrodes are placed in the Lead II position (right atrium to apex) for ECG measurement as follows: one electrode inserted into the right forepaw and the other electrode inserted into the left hind paw.

The body temperature, MAP, ECG, and heart rate are constantly recorded throughout the experiment. Once the critical parameters had stabilized, a 1-2 minute recording is taken to establish the baseline values. Infusion of a compound of the invention or control substance is initiated once baseline values are established. After a 5-minute infusion of compound or control, the suture is pulled tight to ligate the LCA and create ischemia in the left ventricle. The critical parameters are recorded continuously for 20 minutes after ligation, unless the MAP reached the critical level of 20-30 mmHg for at least 3 minutes, in which case the recording is stopped because the animal would be declared deceased and is then sacrificed. The ability of compounds of the invention to prevent arrhythmias and sustain near-normal MAP and HR is scored and compared to control.

### Biological Example 6

### In Vivo Assay for Benign Prostate Hyperplasia (BPH)

The effectiveness of the compounds of the present invention for treating BPH can be demonstrated by the following in vivo assay.

Dogs are dosed orally with compounds of the present invention at oral doses of between 0 mg/kg and 100 mg/kg for a period of 4 weeks. A control group receives placebo. The animals are sacrificed and the prostate glands dissected out, dabbed dry and then weighed. Compounds of the present invention are shown to be efficacious in a dose dependent manner within a range of 5 mg/kg and 100 mg/kg if significantly reducing the weight of the prostate in dogs when compared to the vehicle treated (0 mg/kg) controls.

### Biological Example 7

### In Vivo Assay for Antihypercholesterlemia Efficacy and Antiatherosclerotic Efficacy

The antihypercholesterolemia efficacy of the compounds of this invention can be demonstrated by the following in vivo assay.

Dogs have cardiovascular systems similar to that of humans, making them ideal for studying the effects of medicinal compounds designed to treat cardiovascular disorders.

Dogs are dosed orally at a range of 5 mg/kg to 100 mg/kg daily with compounds of the present invention for a period of 2-4 weeks. After 2 and 4 weeks the animals are bled and their serum collected for total cholesterol analysis and compared to the animals dosed with vehicle alone (0 mg/kg).

The measurement of cholesterol is one of the most common tests performed in the clinical laboratory setting. Simple fluorometric methods for the sensitive quantitation of total cholesterol in plasma or serum are commonly used. In one assay, cholesteryl esters in the sample are first hydrolyzed by cholesterol esterase. All cholesterol, whether previously esterified or existing free in the circulation, is then oxidized by cholesterol oxidase to the corresponding ketone and hydrogen peroxide. ADHP (10-acetyl-3,7-dihydroxyphenoxazine) is utilized as a highly sensitive and stable probe for hydrogen peroxide. Horseradish peroxidase catalyzes the reaction of ADHP with hydrogen peroxide to yield the highly fluorescent product resorufin, which can be monitored using excitation wavelengths of 565-580 nm and emission wavelengths of 585-595 nm.

### Biological Example 8

### In Vivo Assay for Treatment of Pruritis

The compounds of the invention can be evaluated for their activity as antipruritic agents by in vivo test using rodent models. One established model for peripherally elicited pruritus is through the injection of serotonin into the rostral back area (neck) in hairless rats. Prior to serotonin injections (e.g., 2 mg/mL, 50 μL), a dose of a compound of the present invention can be applied systemically through oral, intravenous or intraperitoneal routes or topically to a circular area fixed diameter (e.g. 18 mm). Following dosing, the serotonin injections are given in the area of the topical dosing. After serotonin injection the animal behaviour is monitored by video recording for 20 min-1.5 h, and the number of scratches in this time compared to vehicle treated animals. Thus, application of a compound of the current invention could suppress serotonin-induced scratching in rats.

All of the U.S. patents, U.S. patent application publications, U.S. patent applications, foreign patents, foreign patent applications and non-patent publications referred to in this specification are incorporated herein by reference in their entireties.

Although the foregoing invention has been described in some detail to facilitate understanding, it will be apparent that certain changes and modifications may be practiced within the scope of the appended claims. Accordingly, the described embodiments are to be considered as illustrative and not restrictive, and the invention is not to be limited to the details given herein, but may be modified within the scope and equivalents of the appended claims.

What is claimed is:

1. A compound of formula (I): 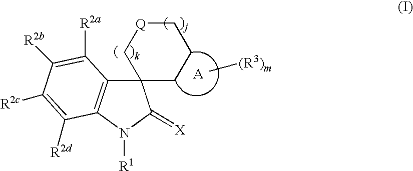
wherein:
j and k are each independently 0, 1, 2 or 3;
m is 0, 1, 2 or 4;
X is O or S; 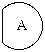
is a fused heterocyclyl ring or a fused heteroaryl ring;
Q is —C(R^{1a})₂—, —O—, —S(O)ₚ— (where p is 0, 1 or 2), —CF₂—, —OC(O)—, —C(O)O—, —C(O)N(R⁵)—, —N(R⁵)— or —N(R⁵)C(O)—;
each R^{1a} is hydrogen or —OR⁵;
or two R^{1a}'s, together with the carbon to which they are attached, form an oxo group;
R¹ is hydrogen, alkyl, alkenyl, alkynyl, haloalkyl, aryl, cycloalkyl, cycloalkylalkyl, heteroaryl, heterocyclyl, —R⁸—C(O)R⁵, —R⁸—C(O)OR⁵, —R⁸—C(O)N(R⁴)R⁵, —S(O)ₜ—R⁵ (where t is 1 or 2), —R⁹—S(O)ₚ—R⁵ (where p is 0, 1 or 2), —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
R⁶ is hydrogen, alkyl, aryl or aralkyl; and
R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
or R⁶ and R⁷, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by one or more substituents selected from the group consisting of —R⁸—OR⁵, —C(O)OR⁵, halo, haloalkyl, alkyl, nitro, cyano, aryl, aralkyl, heterocyclyl and heteroaryl;
or R¹ is —R⁹—N(R¹⁰)¹¹, —R⁹—N(R¹²)C(O)R¹¹, —R⁹—C(O)N(R¹²)R¹¹ or —R⁹—N(R¹⁰)C(O)N(R¹⁰)R¹¹ where:
each R¹⁰ is hydrogen, alkyl, aryl, aralkyl or heteroaryl;
each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—N(R⁴)R⁵, —R⁹—OR⁵ or —R⁹—CN;
R¹² is hydrogen, alkyl, aryl, arylalkyl or —C(O)R⁵;
and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁸—CN, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(═N—CN)N(R⁴)R⁵, wherein each p is independently 0, 1, or 2 and each t is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
or R^{2a} and R^{2b}, together with the carbon ring atoms to which they are directly attached, may form a fused ring selected from cycloalkyl, aryl, heterocyclyl and heteroaryl, and R^{2c} and R^{2d} are as defined above;
or R^{2b} and R^{2c}, together with the carbon ring atoms to which they are directly attached, may form a fused ring selected from cycloalkyl, aryl, heterocyclyl and heteroaryl, and R^{2a} and R^{2d} are as defined above;
or R^{2c} and R^{2d}, together with the carbon ring atoms to which they are directly attached, may form a fused ring selected from cycloalkyl, aryl, heterocyclyl and heteroaryl, and R^{2a} and R^{2b} are as defined above;
each R³ is independently selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁸—CN, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(N═C(R⁴)R⁵)N(R⁴)R⁵, wherein each p is independently 0, 1, or 2 and each t is independently 1 or 2;
and wherein each cycloalkyl, cycloalkylalkyl, aryl, aralkyl, aralkenyl, heterocyclyl, heterocyclylalkyl, heteroaryl and heteroarylalkyl for R³ may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
each R⁴ and R⁵ is independently selected from group consisting of hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl and heteroarylalkyl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain;
as a stereoisomer, enantiomer, tautomer thereof or mixtures thereof;
or a pharmaceutically acceptable salt, N-oxide, solvate or prodrug thereof.

2. The compound of claim 1 wherein X is O and 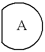
is a fused heteroaryl ring.

3. The compound of claim 2, wherein the compound of formula (I) is a compound of formula (Ia): 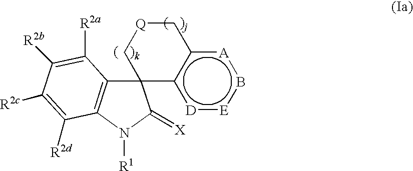
wherein:
j and k are each independently 0, 1, 2 or 3;
Q is —C(R^{1a})H—, —C(O)—, —O—, —S—, —N(R⁵)—, —CF₂—, —C(O)O—, —C(O)N(R⁵)— or —N(R⁵)C(O)—;
A is C(R^{3a}), N or N→O;
B is C(R^{3b}), N or N→O;
D is C(R^{3d}), N or N→O;
E is C(R^{3e}), N or N→O; provided that at least one of A, B, D and E is N or N→O and that no more than two of A, B, C and D are N or N→O at the same time;
or A is C(R^{3a}), B is C(R^{3b}), E is N(H) and D is C(O);
or A is C(R^{3a}), B is C(R^{3b}), D is N(H) and E is C(O);
R^{1a} is hydrogen or —OR⁵;
R¹ is hydrogen, alkyl, alkenyl, alkynyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
R⁶ is hydrogen, alkyl, aryl or aralkyl; and
R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
or R⁶ and R⁷, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
each R¹⁰ is hydrogen, alkyl, aryl or aralkyl;
each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—OR⁵ or —R⁹—CN;
R¹² is hydrogen, alkyl, aryl, aralkyl or —C(O)R⁵;
and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(═N—CN)N(R⁴)R⁵, wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{2a} and R^{2b}, or R^{2b} and R^{2c}, or R^{2c} and R^{2d}, together with the carbon ring atoms to which they are directly attached, may form a fused ring selected from cycloalkyl, aryl, heterocyclyl and heteroaryl;
R^{3a}, R^{3b}, R^{3e} and R^{3d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(N═C(R⁴)R⁵)N(R⁴)R⁵, wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3b}, R^{3e} and R^{3d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3a} and R^{3b}, or R^{3b} and R^{3e}, or R^{3e} and R^{3d}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain;
as a stereoisomer, enantiomer, tautomer thereof or mixtures thereof;
or a pharmaceutically acceptable salt, solvate or prodrug thereof.

4. The compound of claim 3 wherein:
j and k are each independently 0, 1, 2 or 3;
Q is —C(R^{1a})H—, —C(O)—, —O—, —S—, —N(R⁵)—, —CF₂—, —C(O)O—, —C(O)N(R⁵)— or —N(R⁵)C(O)—;
A is C(R^{3a});
B is C(R^{3b}) or N;
E is C(R^{3e});
D is C(R^{3d}) or N, provided that at least one of B and D is N;
R^{1a} is hydrogen or —OR⁵;
R¹ is hydrogen, alkyl, alkenyl, alkynyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
R⁶ is hydrogen, alkyl, aryl or aralkyl; and
R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
or R⁶ and R⁷, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, heterocyclyl and heteroaryl;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
each R¹⁰ is hydrogen, alkyl, aryl or aralkyl;
each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—OR⁵ or —R⁹—CN;
R¹² is hydrogen, alkyl, aryl, aralkyl or —C(O)R⁵;
and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(═N—CN)N(R⁴)R⁵, wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{2a} and R^{2b}, or R^{2b} and R^{2c}, or R^{2c} and R^{2d}, together with the carbon ring atoms to which they are directly attached, may form a fused ring selected from cycloalkyl, aryl, heterocyclyl and heteroaryl;
R^{3a}, R^{3b}, R^{3e} and R^{3d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(N═C(R⁴)R⁵)N(R⁴)R⁵, wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3b}, R^{3e} and R^{3d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3a} and R^{3b}, or R^{3b} and R^{3e}, or R^{3e} and R^{3d}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

5. The compound of claim 4 wherein:
j and k are each independently 0, 1, 2 or 3;
Q is —C(R^{1a})H—, —C(O)—, —O—, —S—, —N(R⁵)—, —CF₂—, —C(O)O—, —C(O)N(R⁵)— or —N(R⁵)C(O)—;
A is C(R^{3a});
B is C(R^{3b});
E is C(R^{3e});
D is N;
R^{1a} is hydrogen or —OR⁵;
R¹ is hydrogen, alkyl, alkenyl, alkynyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
R⁶ is hydrogen, alkyl, aryl or aralkyl; and
R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
or R⁶ and R⁷, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
each R¹⁰ is hydrogen, alkyl, aryl or aralkyl;
each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—OR⁵ or —R⁹—CN;
R¹² is hydrogen, alkyl, aryl, aralkyl or —C(O)R⁵;
and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(═N—CN)N(R⁴)R⁵, wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{2a} and R^{2b}, or R^{2b} and R^{2c}, or R^{2c} and R^{2d}, together with the carbon ring atoms to which they are directly attached, may form a fused ring selected from cycloalkyl, aryl, heterocyclyl and heteroaryl;
R^{3a}, R^{3b}, R^{3e} and R^{3d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(N═C(R⁴)R⁵)N(R⁴)R⁵, wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3b}, R^{3e} and R^{3d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3a} and R^{3b}, or R^{3b} and R^{3e}, or R^{3e} and R^{3d}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

6. The compound of claim 5 wherein:
j is 0 and k is 1;
Q is —C(R^{1a})H—, —O—, —S— or —N(R⁵)—;
A is C(R^{3a});
B is C(R^{3b});
E is C(R^{3e});
D is N;
R^{1a} is hydrogen or —OR⁵;
R¹ is hydrogen, alkyl, alkenyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is —R⁹—C(O)N(R¹²)R¹¹ where:
R¹¹ is hydrogen, alkyl, aryl or aralkyl;
R¹² is hydrogen, alkyl, aryl, aralkyl or —C(O)R⁵;
and wherein each aryl or aralkyl groups for R¹¹ may be optionally substituted by one or more substituents selected from the group consisting of alkyl, cycloalkyl, aryl, aralkyl, halo and haloalkyl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkoxy, halo, haloalkyl, haloalkenyl, haloalkoxy, cycloalkyl, cycloalkylalkyl, aryl, aralkyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴ and —R⁸—C(O)N(R⁴)R⁵;
and wherein each of the cycloalkyl, cycloalkylalkyl, aryl, aralkyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein m is 0, 1, or 2 and n is 1 or 2;
R^{3a}, R^{3b} and R^{3e} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴,
wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3b} and R^{3e} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3a} and R^{3b} or R^{3b} and R^{3e}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain; and
each R⁹ is a straight or branched alkylene chain.

7. The compound of claim 6 wherein:
j is 0 and k is 1;
Q is —O—;
A is C(R^{3a});
B is C(R^{3b});
E is C(R^{3e});
D is N;
R¹ is hydrogen, alkyl, alkenyl, haloalkyl, —R⁸—OR⁵ or —R⁸—CN;
or R¹ is —R⁹—C(O)N(R¹²)R¹¹ where:
R¹¹ is hydrogen, alkyl, aryl or aralkyl;
R¹² is hydrogen, alkyl, aryl, aralkyl or —C(O)R⁵;
and wherein each aryl or aralkyl groups for R¹¹ may be optionally substituted by one or more substituents selected from the group consisting of alkyl, cycloalkyl, aryl, aralkyl, halo and haloalkyl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkoxy, halo, haloalkyl, heteroaryl, —R⁸—OR⁵, —R⁸—N(R⁴)R⁵, —R⁸—C(O)R⁴, —R⁸—C(O)OR⁴ and —R⁸—C(O)N(R⁴)R⁵;
R^{3a}, R^{3b} and R^{3e} are each independently selected from the group consisting of hydrogen, alkyl, alkoxy, halo, haloalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴;
or R^{3a} and R^{3b} or R^{3b} and R^{3e}, together with the carbon ring atoms to which they are directly attached, may form a fused heterocyclyl ring;
each R⁴ and R⁵ is independently selected from group consisting of hydrogen, alkyl, haloalkyl, alkoxyalkyl, cycloalkyl, cycloalkylalkyl, aryl, aralkyl, heterocyclyl and heteroaryl; and
each R⁸ is a direct bond or a straight or branched alkylene chain.

8. The compound of claim 7 wherein:
j is 0 and k is 1;
Q is —O—;
A is C(R^{3a});
B is C(R^{3b});
E is C(R^{3e});
D is N;
R¹ is hydrogen or heteroarylalkyl where the heteroarylalkyl group is optionally substituted by one or more substituents selected from the group consisting of alkyl, halo, haloalkyl, —R⁸—OR⁵ and —R⁸—C(O)OR⁵;
R^{2a}, R^{2b}, R^{2c} and R^{2d} are each independently selected from the group consisting of hydrogen, alkyl, halo, haloalkyl and heteroaryl;
R^{3a}, R^{3b} and R^{3e} are each independently selected from the group consisting of hydrogen, alkyl, alkoxy, halo, haloalkyl and —R⁸—OR⁵;
or R^{3a} and R^{3b} or R^{3b} and R^{3e}, together with the carbon ring atoms to which they are directly attached, may form a fused ring selected from heterocyclyl;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

9. The compound of claim 8 wherein:
j is 0 and k is 1;
Q is —O—;
A is C(R^{3a});
B is C(R^{3b});
E is C(R^{3e});
D is N;
R¹ is hydrogen;
R^{2a}, R^{2b}, R^{2c} and R^{2d} are each independently selected from the group consisting of hydrogen, alkyl, halo and haloalkyl;
R^{3a}, R^{3b} and R^{3e} are each independently selected from the group consisting of hydrogen, alkyl, alkoxy, halo, haloalkyl and —R⁸—OR⁵;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

10. The compound of claim 9 selected from the group consisting of:
4′-bromo-5-methoxyspiro[furo[3,2-b]pyridine-3,3′-indol]-2′(1′H)-one; and
5-methoxyspiro[furo[3,2-b]pyridine-3,3′-indol]-2′(1′H)-one.

11. The compound of claim 8 wherein:
j is 0 and k is 1;
Q is —O—;
A is C(R^{3a});
B is C(R^{3b});
E is C(R^{3e});
D is N;
R¹ is heteroarylalkyl where the heteroarylalkyl group is optionally substituted by one or more substituents selected from the group consisting of alkyl, halo, haloalkyl, —R⁸—OR⁵ and —R⁸—C(O)OR⁵;
R^{2a}, R^{2b}, R^{2c} and R^{2d} are each independently selected from the group consisting of hydrogen, alkyl, halo, haloalkyl and heteroaryl;
R^{3a}, R^{3b} and R^{3e} are each independently selected from the group consisting of hydrogen, alkyl, alkoxy, halo, haloalkyl and —R⁸—OR⁵;
or R^{3a} and R^{3b} or R^{3b} and R^{3e}, together with the carbon ring atoms to which they are directly attached, may form a fused ring selected from heterocyclyl;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

12. The compound of claim 11 selected from the group consisting of:
4′-bromo-5-methoxy-1′-{[5-(trifluoromethyl)furan-2-yl]methyl}spiro[furo[3,2-b]pyridine-3,3′-indol]-2′(1′H)-one;
5-methoxy-1′-{[5-(trifluoromethyl)furan-2-yl]methyl}spiro[furo[3,2-b]pyridine-3,3′-indol]-2′(1′H)-one;
4′-furan-3-yl-5-methoxy-1′-{[5-(trifluoromethyl)furan-2-yl]methyl}spiro[furo[3,2-b]pyridine-3,3′-indol]-2′(1′H)-one;
1′-{[5-(trifluoromethyl)furan-2-yl]methyl}spiro[1,3-dioxolo[4,5-b]furo[2,3-e]pyridine-5,3′-indol]-2′(1′H)-one;
5′-fluoro-5-methoxy-1′{[5-(trifluoromethyl)furan-2-yl]methyl}spiro[furo[3,2-b]pyridine-3,3′-indol]-2′(1′H)-one;
4′-chloro-5-methoxy-1′-{[5-(trifluoromethyl)furan-2-yl]methyl}spiro[furo[3,2-b]pyridine-3,3′-indol]-2′(1′H)-one;
1′-[(5-chloro-1-methyl-1H-imidazol-2-yl)methyl]-5-methoxyspiro[furo[3,2-b]pyridine-3,3′-indol]-2′(1′H)-one;
5-methoxy-1′-(pyridin-2-ylmethyl)spiro[furo[3,2-b]pyridine-3,3′-indol]-2′(1′H)-one;
4′-bromo-1′-[(2-isopropyl-1,3-thiazol-5-yl)methyl]-5-methoxyspiro[furo[3,2-b]pyridine-3,3′-indol]-2′(1′H)-one;
1′-[(5-chlorothiophen-2-yl)methyl]-5-methoxyspiro[furo[3,2-b]pyridine-3,3′-indol]-2′(1′H)-one; and
5-methoxy-1′-{[2-(1-methylethyl)-1,3-thiazol-4-yl]methyl}spiro[furo[3,2-b]pyridine-3,3′-indol]-2′(1′H)-one.

13. The compound of claim 7 wherein:
j is 0 and k is 1;
Q is —O—;
A is C(R^{3a});
B is C(R^{3b});
E is C(R^{3e});
D is N;
R¹ is heterocyclylalkyl where the heterocyclylalkyl group is optionally substituted by one or more substituents selected from the group consisting of alkyl, halo, haloalkyl, —R⁸—OR⁵ and —R⁸—C(O)OR⁵;
R^{2a}, R^{2b}, R^{2c} and R^{2d} are each independently selected from the group consisting of hydrogen, alkyl, halo, haloalkyl and heteroaryl;
R^{3a}, R^{3b} and R^{3e} are each independently selected from the group consisting of hydrogen, alkyl, alkoxy, halo, haloalkyl and —R⁸—OR⁵;
or R^{3a} and R^{3b} or R^{3b} and R^{3e}, together with the carbon ring atoms to which they are directly attached, may form a fused ring selected from heterocyclyl;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

14. The compound of claim 13 selected from the group consisting of:
5-methoxy-1′-(piperidin-4-ylmethyl)spiro[furo[3,2-b]pyridine-3,3′-indol]-2′(1′H)-one;
tert-butyl 4-[(4′-bromo-5-methoxy-2′-oxospiro[furo[3,2-b]pyridine-3,3′-indol]-1′(2′H)-yl)methyl]piperidine-1-carboxylate; and
5-methoxy-1-[(1-methylpiperidin-4-yl)methyl]spiro[furo[3,2-b]pyridine-3,3′-indol]-2′(1′H)-one.

15. The compound of claim 7 wherein:
j is 0 and k is 1;
Q is —O—;
A is C(R^{3a});
B is C(R^{3b});
E is C(R^{3e});
D is N;
R¹ is —R⁹—C(O)N(R¹²)R¹¹ where:
R¹¹ is hydrogen, alkyl, aryl or aralkyl;
R¹² is hydrogen, alkyl, aryl, aralkyl or —C(O)R⁵;
and wherein each aryl or aralkyl groups for R¹¹ may be optionally substituted by one or more substituents selected from the group consisting of alkyl, cycloalkyl, aryl, aralkyl, halo and haloalkyl;
R^{2a}, R^{2b}, R^{2c} and R^{2d} are each independently selected from the group consisting of hydrogen, alkyl, halo, haloalkyl and heteroaryl;
R^{3a}, R^{3b} and R^{3e} are each independently selected from the group consisting of hydrogen, alkyl, alkoxy, halo, haloalkyl and —R⁸—OR⁵;
or R^{3a} and R^{3b} or R^{3b} and R^{3e}, together with the carbon ring atoms to which they are directly attached, may form a fused ring selected from heterocyclyl;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

16. The compound of claim 15 which is N-(2-fluorophenyl)-2-(5-methoxy-2′-oxospiro[furo[3,2-b]pyridine-3,3′-indol]-1′(2′H)-yl)acetamide.

17. The compound of claim 4 wherein:
j and k are each independently 0, 1, 2 or 3;
Q is —C(R^{1a})H—, —C(O)—, —O—, —S—, —N(R⁵)—, —CF₂—, —C(O)O—, —C(O)N(R⁵)— or —N(R⁵)C(O)—;
A is C(R^{3a});
B is N;
E is C(R^{3e});
D is C(R^{3d});
R^{1a} is hydrogen or —OR⁵;
R¹ is hydrogen, alkyl, alkenyl, alkynyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
R⁶ is hydrogen, alkyl, aryl or aralkyl; and
R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
or R⁶ and R⁷, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is —R⁹—N(R¹⁰)R¹¹, —R⁹—N(R¹²)C(O)R¹¹ or —R⁹—N(R¹⁰)C(O)N(R¹⁰)R¹¹ where:
each R¹⁰ is hydrogen, alkyl, aryl or aralkyl;
each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—OR⁵ or —R⁹—CN;
R¹² is hydrogen, alkyl, aryl, aralkyl or —C(O)R⁵;
and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(═N—CN)N(R⁴)R⁵, wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{2a} and R^{2b}, or R^{2b} and R^{2c}, or R^{2c} and R^{2d}, together with the carbon ring atoms to which they are directly attached, may form a fused ring selected from cycloalkyl, aryl, heterocyclyl and heteroaryl;
R^{3a}, R^{3e} and R^{3d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(N═C(R⁴)R⁵)N(R⁴)R⁵, wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3e} and R^{3d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3e} and R^{3d}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

18. The compound of claim 17 wherein:
j is 0 and k is 1;
Q is —C(R^{1a})H—, —O—, —S— or —N(R⁵)—;
A is C(R^{3a});
B is N;
E is C(R^{3e});
D is C(R^{3d});
R^{1a} is hydrogen or —OR⁵;
R¹ is hydrogen, alkyl, alkenyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkoxy, halo, haloalkyl, haloalkenyl, haloalkoxy, cycloalkyl, cycloalkylalkyl, aryl, aralkyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴ and —R⁸—C(O)N(R⁴)R⁵;
and wherein each of the cycloalkyl, cycloalkylalkyl, aryl, aralkyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein m is 0, 1, or 2 and n is 1 or 2;
R^{3a}, R^{3e} and R^{3d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴,
and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3e} and R^{3d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3e} and R^{3d}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain; and
each R⁹ is a straight or branched alkylene chain.

19. The compound of claim 18 wherein:
j is 0 and k is 1;
Q is —O—;
A is C(R^{3a});
B is N;
E is C(R^{3e});
D is C(R^{3d});
R¹ is hydrogen, alkyl, alkenyl, haloalkyl, —R⁸—OR⁵ or —R⁸—CN;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkoxy, halo, haloalkyl, heteroaryl, —R⁸—OR⁵, —R⁸—N(R⁴)R⁵, —R⁸—C(O)R⁴, —R⁸—C(O)OR⁴ and —R⁸—C(O)N(R⁴)R⁵;
R^{3a}, R^{3e} and R^{3d} are each independently selected from the group consisting of hydrogen, alkyl, alkoxy, halo, haloalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴;
or R^{3e} and R^{3d}, together with the carbon ring atoms to which they are directly attached, may form a fused heterocyclyl ring;
each R⁴ and R⁵ is independently selected from group consisting of hydrogen, alkyl, haloalkyl, alkoxyalkyl, cycloalkyl, cycloalkylalkyl, aryl, aralkyl, heterocyclyl and heteroaryl; and
each R⁸ is a direct bond or a straight or branched alkylene chain.

20. The compound of claim 19 wherein:
j is 0 and k is 1;
Q is —O—;
A is C(R^{3a});
B is N;
E is C(R^{3e});
D is C(R^{3d});
R¹ is hydrogen;
R^{2a}, R^{2b}, R^{2c} and R^{2d} are each independently selected from the group consisting of hydrogen, alkyl, halo, haloalkyl and heteroaryl;
R^{3a}, R^{3e} and R^{3d} are each independently selected from the group consisting of hydrogen, alkyl, alkoxy, halo, haloalkyl and —R⁸—OR⁵;
or R^{3e} and R^{3d}, together with the carbon ring atoms to which they are directly attached, may form a fused ring selected from heterocyclyl;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

21. The compound of claim 20 which is 5-methoxyspiro[furo[2,3-c]pyridine-3,3′-indol]-2′(1′H)-one.

22. The compound of claim 19 wherein:
j is 0 and k is 1;
Q is —O—;
A is C(R^{3a});
B is N;
E is C(R^{3e});
D is C(R^{3d});
R¹ is heteroarylalkyl where the heteroarylalkyl group is optionally substituted by one or more substituents selected from the group consisting of alkyl, halo, haloalkyl, —R⁸—OR⁵ and —R⁸—C(O)OR⁵;
R^{2a}, R^{2b}, R^{2c} and R^{2d} are each independently selected from the group consisting of hydrogen, alkyl, halo, haloalkyl and heteroaryl;
R^{3a}, R^{3e} and R^{3d} are each independently selected from the group consisting of hydrogen, alkyl, alkoxy, halo, haloalkyl and —R⁸—OR⁵;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

23. The compound of claim 22 which is 5-methoxy-1′-{[5-(trifluoromethyl)furan-2-yl]methyl}spiro[furo[2,3-c]pyridine-3,3′-indol]-2′(1′H)-one;

24. The compound of claim 19 wherein:
j is 0 and k is 1;
Q is —O—;
A is C(R^{3a});
B is N;
E is C(R^{3e});
D is C(R^{3d});
R¹ is heterocyclylalkyl where the heterocyclylalkyl group is optionally substituted by one or more substituents selected from the group consisting of alkyl, halo, haloalkyl, —R⁸—OR⁵ and —R⁸—C(O)OR⁵;
R^{2a}, R^{2b}, R^{2c} and R^{2d} are each independently selected from the group consisting of hydrogen, alkyl, halo, haloalkyl and heteroaryl;
R^{3a}, R^{3e} and R^{3d} are each independently selected from the group consisting of hydrogen, alkyl, alkoxy, halo, haloalkyl and —R⁸—OR⁵;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

25. The compound of claim 24 selected from the group consisting of:
5-methoxy-1′-(tetrahydro-2H-pyran-4-ylmethyl)spiro[furo[2,3-c]pyridine-3,3′-indol]-2′(1′H)-one;
5-methoxy-1′-[(2S)-tetrahydrofuran-2-ylmethyl]spiro[furo[2,3-c]pyridine-3,3′-indol]-2′(1′H)-one; and
5-methoxy-1′-[(2R)-tetrahydrofuran-2-ylmethyl]spiro[furo[2,3-c]pyridine-3,3′-indol]-2′(1′H)-one.

26. The compound of claim 19 wherein:
j is 0 and k is 1;
Q is —O—;
A is C(R^{3a});
B is N;
E is C(R^{3e});
D is C(R^{3d});
R¹ is aralkyl;
R^{2a}, R^{2b}, R^{2c} and R^{2d} are each independently selected from the group consisting of hydrogen, alkyl, halo, haloalkyl and heteroaryl;
R^{3a}, R^{3e} and R^{3d} are each independently selected from the group consisting of hydrogen, alkyl, alkoxy, halo, haloalkyl and —R⁸—OR⁵;
or R^{3e} and R^{3d}, together with the carbon ring atoms to which they are directly attached, may form a fused ring selected from heterocyclyl;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

27. The compound of claim 26 which is 1′-(diphenylmethyl)-5-methoxyspiro[furo[2,3-c]pyridine-3,3′-indol]-2′(1′H)-one.

28. The compound of claim 3 wherein:
j and k are each independently 0, 1, 2 or 3;
Q is —C(R^{1a})H—, —C(O)—, —O—, —S—, —N(R⁵)—, —CF₂—, —C(O)O—, —C(O)N(R⁵)— or —N(R⁵)C(O)—;
A is C(R^{3a});
B is C(R^{3b});
E is N;
D is C(R^{3d});
R^{1a} is hydrogen or —OR⁵;
R¹ is hydrogen, alkyl, alkenyl, alkynyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
R⁶ is hydrogen, alkyl, aryl or aralkyl; and
R⁷ is hydrogen, alkyl, haloalkyl, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
or R⁶ and R⁷, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is —R⁹—N(R¹⁰)R¹¹, —R⁹—N(R¹²)C(O)R¹¹ or —R⁹—N(R¹⁰)C(O)N(R¹⁰)R¹¹ where:
each R¹⁰ is hydrogen, alkyl, aryl or aralkyl;
each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—OR⁵ or —R⁹—CN;
R¹² is hydrogen, alkyl, aryl, arylalkyl or —C(O)R⁵;
and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(═N—CN)N(R⁴)R⁵, wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{2a} and R^{2b}, R^{2b} and R^{2c}, or R^{2c} and R^{2d}, together with the carbon ring atoms to which they are directly attached, may form a fused ring selected from cycloalkyl, aryl, heterocyclyl and heteroaryl;
R^{3a}, R^{3b} and R^{3d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(N═C(R⁴)R⁵)N(R⁴)R⁵, wherein each m is independently 0, 1, or 2 and each n is independently 1 or 2,
and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3b} and R^{3d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3a} and R^{3b}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

29. The compound of claim 28 wherein:
j is 0 and k is 1;
Q is —O—;
A is C(R^{3a});
B is C(R^{3b});
E is N;
D is C(R^{3d});
R¹ is hydrogen, alkyl, alkenyl, alkynyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
R^{2a}, R^{2b}, R^{2c} and R^{2d} are each independently selected from the group consisting of hydrogen, alkyl, halo, haloalkyl and heteroaryl;
R^{3a}, R^{3b} and R^{3d} are each independently selected from the group consisting of hydrogen, alkyl, alkoxy, halo, haloalkyl and —R⁸—OR⁵;
or R^{3a} and R^{3b}, together with the carbon ring atoms to which they are directly attached, may form a fused ring selected from heterocyclyl;
each R⁴ and R⁵ is independently selected from group consisting of hydrogen, alkyl, haloalkyl, alkoxyalkyl, aryl and aralkyl;
each R⁸ is a direct bond or a straight or branched alkylene chain; and
each R⁹ is a straight or branched alkylene chain, a straight or branched alkenylene chain or a straight or branched alkynylene chain.

30. The compound of claim 29 wherein:
j is 0 and k is 1;
Q is —O—;
A is C(R^{3a});
B is C(R^{3b});
E is N;
D is C(R^{3d});
R¹ is hydrogen or alkyl;
R^{2a}, R^{2b}, R^{2c} and R^{2d} are each independently selected from the group consisting of hydrogen, alkyl, halo, haloalkyl and heteroaryl;
R^{3a}, R^{3b} and R^{3d} are each independently selected from the group consisting of hydrogen, alkyl, alkoxy, halo, haloalkyl and —R⁸—OR⁵;
each R⁴ and R⁵ is independently selected from group consisting of hydrogen, alkyl, haloalkyl, alkoxyalkyl, aryl and aralkyl; and
each R⁸ is a direct bond or a straight or branched alkylene chain.

31. The compound of claim 30 which is 1′-pentylspiro[furo[3,2-c]pyridine-3,3′-indol]-2′(1′H)-one.

32. The compound of claim 3 wherein:
j and k are each independently 0, 1, 2 or 3;
Q is —C(R^{1a})H—, —C(O)—, —O—, —S—, —N(R⁵)—, —CF₂—, —C(O)O—, —C(O)N(R⁵)— or —N(R⁵)C(O)—;
A is C(R^{3a});
B is C(R^{3b});
E is N→O;
D is C(R^{3d});
R^{1a} is hydrogen or —OR⁵;
R¹ is hydrogen, alkyl, alkenyl, alkynyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁵—O—R⁹—OR⁵;
or R¹ is aralkyl substituted by —C(O)N(R⁶)R⁷ where:
R⁶ is hydrogen, alkyl, aryl or aralkyl; and
R⁷ is hydrogen, alkyl, haloalkyl, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
or R⁶ and R⁷, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
each R¹⁰ is hydrogen, alkyl, aryl or aralkyl;
each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—OR⁵ or —R⁹—CN;
R¹² is hydrogen, alkyl, aryl, aralkyl or —C(O)R⁵;
and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(═N—CN)N(R⁴)R⁵, wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{2a} and R^{2b}, or R^{2b} and R^{2c}, or R^{2c} and R^{2d}, together with the carbon ring atoms to which they are directly attached, may form a fused ring selected from cycloalkyl, aryl, heterocyclyl and heteroaryl;
R^{3a}, R^{3b} and R^{3d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(N═C(R⁴)R⁵)N(R⁴)R⁵, wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3b} and R^{3d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3a} and R^{3b}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

33. The compound of claim 32 wherein:
j is 0 and k is 1;
Q is —C(R^{1a})H—, —O—, —S— or —N(R⁵)—;
A is C(R^{3a});
B is C(R^{3b});
E is N→O;
D is C(R^{3d});
R^{1a} is hydrogen or —OR⁵;
R¹ is hydrogen, alkyl, alkenyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
R^{2a}, R^{2b}, R^{2a} and R^{2d} are each independently selected from the group consisting of hydrogen, alkyl, alkenyl, alkoxy, halo, haloalkyl, haloalkenyl, haloalkoxy, cycloalkyl, cycloalkylalkyl, aryl, aralkyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴ and —R⁸—C(O)N(R⁴)R⁵ and —N(R⁵)S(O)ₘR⁴ wherein n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
R^{3a}, R^{3b} and R^{3d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴,
and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3b} and R^{3d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

34. The compound of claim 33 wherein:
j is 0 and k is 1;
Q is —O—;
A is C(R^{3a});
B is C(R^{3b});
E is N→O;
D is C(R^{3d});
R¹ is hydrogen, alkyl, alkenyl, or haloalkyl;
R^{2a}, R^{2b}, R^{2c} and R^{2d} are each independently selected from the group consisting of hydrogen, alkyl, alkenyl, alkoxy, halo, haloalkyl, —R⁸—OR⁵, —R⁸—N(R⁴)R⁵, —R⁸—C(O)R⁴, —R⁸—C(O)OR⁴ and —R⁸—C(O)N(R⁴)R⁵;
R^{3a}, R^{3b} and R^{3d} are each independently selected from the group consisting of hydrogen, alkyl, alkenyl, alkoxy, halo, haloalkyl, haloalkoxy, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain.

35. The compound of claim 34 which is 1′-pentylspiro[furo[3,2-c]pyridine-3,3′-indol]-2′(1′H)-one 5-oxide.

36. The compound of claim 3 wherein:
j and k are each independently 0, 1, 2 or 3;
Q is —C(R^{1a})H—, —C(O)—, —O—, —S—, —N(R⁵)—, —CF₂—, —C(O)O—, —C(O)N(R⁵)— or —N(R⁵)C(O)—;
A is C(R^{3a});
B is C(R^{3b});
E is C(R^{3e});
D is N→O;
R^{1a} is hydrogen or —OR⁵;
R¹ is hydrogen, alkyl, alkenyl, alkynyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
R⁶ is hydrogen, alkyl, aryl or aralkyl; and
R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
or R⁶ and R⁷, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
each R¹⁰ is hydrogen, alkyl, aryl or aralkyl;
each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—OR⁵ or —R⁹—CN;
R¹² is hydrogen, alkyl, aryl, aralkyl or —C(O)R⁵;
and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(═N—CN)N(R⁴)R⁵, wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₘR⁴, wherein each m is independently 0, 1, or 2 and each n is independently 1 or 2;
or R^{2a} and R^{2b}, R^{2b} and R^{2c}, or R² and R^{2d}, together with the carbon ring atoms to which they are directly attached, may form a fused ring selected from cycloalkyl, aryl, heterocyclyl and heteroaryl;
R^{3a}, R^{3b} and R^{3e} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(N═C(R⁴)R⁵)N(R⁴)R⁵, wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3b} and R^{3e} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3a} and R^{3b}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

37. The compound of claim 36 wherein:
j is 0 and k is 1;
Q is —C(R^{1a})H—, —O—, —S— or —N(R⁵)—;
A is C(R^{3a});
B is C(R^{3b});
E is C(R^{3e});
D is N→O;
R^{1a} is hydrogen or —OR⁵;
R¹ is hydrogen, alkyl, alkenyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkoxy, halo, haloalkyl, haloalkenyl, haloalkoxy, cycloalkyl, cycloalkylalkyl, aryl, aralkyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)S(O)ₙR⁴ wherein n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
R^{3a}, R^{3b} and R^{3e} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴,
and wherein each of the cycloalkyl, cycloalkylalkyl, aryl, aralkyl, aralkenyl, heterocyclyl, heterocyclylalkyl, heteroaryl and heteroarylalkyl groups for R^{3a}, R^{3b} and R^{3e} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

38. The compound of claim 37 wherein:
j is 0 and k is 1;
Q is —O—;
A is C(R^{3a});
B is C(R^{3b});
E is C(R^{3e});
D is N→O;
R¹ is hydrogen, alkyl, alkenyl or haloalkyl;
or R¹ is heteroarylalkyl where the heterocyclylalkyl or the heteroarylalkyl group is optionally substituted by one or more substituents selected from the group consisting of alkyl, halo, haloalkyl, —R⁸—OR⁵, and —R⁸—C(O)OR⁵;
R^{2a}, R^{2b}, R^{2c} and R^{2d} are each independently selected from the group consisting of hydrogen, alkyl, alkenyl, alkoxy, halo, haloalkyl, —R⁸—OR⁵, —R⁸—N(R⁴)R⁵, —R⁸—C(O)R⁴, —R⁸—C(O)OR⁴ and —R⁸—C(O)N(R⁴)R⁵;
R^{3a}, R^{3b} and R^{3e} are each independently selected from the group consisting of hydrogen, alkyl, alkenyl, alkoxy, halo, haloalkyl, haloalkoxy, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain.

39. The compound of claim 38 which is 5-methoxy-1′-{[5-(trifluoromethyl)-2-furyl]methyl}spiro[furo[3,2-b]pyridine-3,3′-indol]-2′(1′H)-one 4-oxide.

40. The compound of claim 3 wherein:
j and k are each independently 0, 1, 2 or 3;
Q is —C(R^{1a})H—, —C(O)—, —O—, —S—, —N(R⁵)—, —CF₂—, —C(O)O—, —C(O)N(R⁵)— or —N(R⁵)C(O)—;
A is C(R^{3a}), B is C(R^{3b}), E is N(H) and D is C(O);
or A is C(R^{3a}), B is C(R^{3b}), D is N(H) and E is C(O);
R^{1a} is hydrogen or —OR⁵;
R¹ is hydrogen, alkyl, alkenyl, alkynyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
R⁶ is hydrogen, alkyl, aryl or aralkyl; and
R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
or R⁶ and R⁷, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by —R⁸—OR⁵, —C(O)OR⁵, halo, haloalkyl, alkyl, nitro, cyano, aryl (optionally substituted by cyano), aralkyl (optionally substituted by one or more alkyl groups), heterocyclyl or heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
each R¹⁰ is hydrogen, alkyl, aryl or aralkyl;
each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—OR⁵ or —R⁹—CN;
R¹² is hydrogen, alkyl, aryl, aralkyl or —C(O)R⁵;
and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(═N—CN)N(R⁴)R⁵, wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{2a} and R^{2b}, or R^{2b} and R^{2c}, or R^{2c} and R^{2d}, together with the carbon ring atoms to which they are directly attached, may form a fused ring selected from cycloalkyl, aryl, heterocyclyl and heteroaryl;
R^{3a} and R^{3b} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —N═C(R⁴)R⁵, —S(O)ₘR⁴, —R⁸—C(O)R⁴, —C(S)R⁴, —C(R⁴)₂C(O)R⁵, —R⁸—C(O)OR⁴, —C(S)OR⁴, —R⁸—C(O)N(R⁴)R⁵, —C(S)N(R⁴)R⁵, —N(R⁵)C(O)R⁴, —N(R⁵)C(S)R⁴, —N(R⁵)C(O)OR⁴, —N(R⁵)C(S)OR⁴, —N(R⁵)C(O)N(R⁴)R⁵, —N(R⁵)C(S)N(R⁴)R⁵, —N(R⁵)S(O)ₙR⁴, —N(R⁵)S(O)ₙN(R⁴)R⁵, —N(R⁵)C(═NR⁵)N(R⁴)R⁵ and —N(R⁵)C(N═C(R⁴)R⁵)N(R⁴)R⁵, wherein each m is independently 0, 1, or 2 and each n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{3a} and R^{3b} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
or R^{3a} and R^{3b}, together with the carbon ring atoms to which they are directly attached, may form a fused ring selected from cycloalkyl, heterocyclyl, aryl or heteroaryl;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

41. The compound of claim 40 wherein:
j is 0 and k is 1;
Q is —C(R^{1a})H—, —O—, —S— or —N(R⁵)—;
A is C(R^{3a}), B is C(R^{3b}), E is N(H) and D is C(O);
or A is C(R^{3a}), B is C(R^{3b}), D is N(H) and E is C(O);
R^{1a} is hydrogen or —OR⁵;
R¹ is hydrogen, alkyl, alkenyl, haloalkyl, aryl, cycloalkyl, cycloalkylalkyl, heteroaryl, heterocyclyl, —R⁸—OR⁵, —R⁸—CN, —R⁹—P(O)(OR⁵)₂ or —R⁹—O—R⁹—OR⁵;
R^{2a}, R^{2b}, R^{2c} and R^{2d} are each independently selected from the group consisting of hydrogen, alkyl, alkenyl, alkoxy, halo, haloalkyl, haloalkenyl, haloalkoxy, cycloalkyl, cycloalkylalkyl, aryl, aralkyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)S(O)ₙR⁴ wherein n is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
R^{3a} and R^{3b} are each independently selected from the group consisting of hydrogen, alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴,
and wherein each of the cycloalkyl, cycloalkylalkyl, aryl, aralkyl, aralkenyl, heterocyclyl, heterocyclylalkyl, heteroaryl and heteroarylalkyl groups for R^{3a} and R^{3b} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₘR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₙR⁴, wherein each m is independently 0, 1, or 2 and each n is independently 1 or 2;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain.

42. The compound of claim 41 wherein:
j is 0 and k is 1;
Q is —O—;
A is C(R^{3a}), B is C(R^{3b}), E is N(H) and D is C(O);
or A is C(R^{3a}), B is C(R^{3b}), D is N(H) and E is C(O);
R¹ is hydrogen, alkyl, alkenyl or haloalkyl;
R^{2a}, R^{2b}, R^{2c} and R^{2d} are each independently selected from the group consisting of hydrogen, alkyl, alkenyl, alkoxy, halo, haloalkyl, —R⁸—OR⁵, —R⁸—N(R⁴)R⁵, —R⁸—C(O)R⁴, —R⁸—C(O)OR⁴ and —R⁸—C(O)N(R⁴)R⁵;
R^{3a}, R^{3b} and R^{3e} are each independently selected from the group consisting of hydrogen, alkyl, alkenyl, alkoxy, halo, haloalkyl, haloalkoxy, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain.

43. The compound of claim 42 wherein:
j is 0 and k is 1;
Q is —O—;
A is C(R^{3a}), B is C(R^{3b}), E is N(H) and D is C(O);
R¹ is hydrogen, alkyl, alkenyl or haloalkyl;
R^{2a}, R^{2b}, R^{2c} and R^{2d} are each independently selected from the group consisting of hydrogen, alkyl, alkenyl, alkoxy, halo, haloalkyl, —R⁸—OR⁵, —R⁸—N(R⁴)R⁵, —R⁸—C(O)R⁴, —R⁸—C(O)OR⁴ and —R⁸—C(O)N(R⁴)R⁵;
R^{3a}, R^{3b} and R^{3e} are each independently selected from the group consisting of hydrogen, alkyl, alkenyl, alkoxy, halo, haloalkyl, haloalkoxy, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain.

44. The compound of claim 43 which is 1′-pentylspiro[furo[3,2-c]pyridine-3,3′-indole]-2′,4(1′,5H)-dione.

45. The compound of claim 42 wherein:
j is 0 and k is 1;
Q is —O—;
A is C(R^{3a}), B is C(R^{3b}), D is N(H) and E is C(O);
R¹ is hydrogen, alkyl, alkenyl or haloalkyl;
R^{2a}, R^{2b}, R^{2c} and R^{2d} are each independently selected from the group consisting of hydrogen, alkyl, alkenyl, alkoxy, halo, haloalkyl, —R⁸—OR⁵, —R⁸—N(R⁴)R⁵, —R⁸—C(O)R⁴, —R⁸—C(O)OR⁴ and —R⁸—C(O)N(R⁴)R⁵;
R^{3a}, R^{3b} and R^{3e} are each independently selected from the group consisting of hydrogen, alkyl, alkenyl, alkoxy, halo, haloalkyl, haloalkoxy, —R⁸—CN, —R⁸—N(O)₂, —R⁸—OR⁵, —R⁸—N(R⁴)R⁵, —R⁸—C(O)R⁴, —R⁸—C(O)OR⁴, —R⁸—C(O)N(R⁴)R⁵ and —N(R⁵)C(O)R⁴;
each R⁴ and R⁵ is independently selected from group consisting of hydrogen, alkyl, alkenyl, alkynyl, haloalkyl, alkoxyalkyl, cycloalkyl, cycloalkylalkyl, aryl, aralkyl, heterocyclyl and heteroaryl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain.

46. The compound of claim 45 which is 1′-{[5-(trifluoromethyl)-2-furyl]methyl}spiro[furo[3,2-b]pyridine-3,3′-indole]-2′,5(1′H,4H)-dione.

47. A method of treating, preventing or ameliorating a disease or a condition in a mammal selected from the group consisting of pain, depression, cardiovascular diseases, respiratory diseases, and psychiatric diseases, and combinations thereof, wherein the method comprises administering to the mammal in need thereof a therapeutically effective amount of a compound of formula (I): 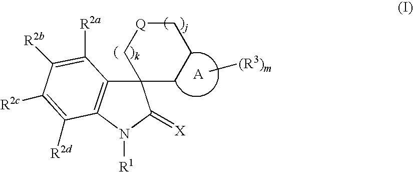
wherein:
j and k are each independently 0, 1, 2 or 3;
m is 0, 1, 2 or 4;
X is O or S; 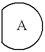
is a fused heterocyclyl ring or a fused heteroaryl ring;
Q is —C(R^{1a})₂—, —O—, —S(O)ₚ— (where p is 0, 1 or 2), —CF₂—, —OC(O)—, —C(O)O—, —C(O)N(R⁵)—, —N(R⁵)— or —N(R⁵)C(O)—;
each R^{1a} is hydrogen or —OR⁵;
or two R^{1a}'s, together with the carbon to which they are attached, form an oxo group;
R¹ is hydrogen, alkyl, alkenyl, alkynyl, haloalkyl, aryl, cycloalkyl, cycloalkylalkyl, heteroaryl, heterocyclyl, —R⁸—C(O)R⁵, —R⁸—C(O)OR⁵, —R⁸—C(O)N(R⁴)R⁵, —S(O)ₜ—R⁵ (where t is 1 or 2), —R⁹—S(O)ₚ—R⁵ (where p is 0, 1 or 2), —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
R⁶ is hydrogen, alkyl, aryl or aralkyl; and
R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
or R⁶ and R⁷, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by one or more substituents selected from the group consisting of —R⁸—OR⁵, —C(O)OR⁵, halo, haloalkyl, alkyl, nitro, cyano, aryl, aralkyl, heterocyclyl and heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
each R¹⁰ is hydrogen, alkyl, aryl, aralkyl or heteroaryl;
each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—N(R⁴)R⁵, —R⁹—OR⁵ or —R⁹—CN;
R¹² is hydrogen, alkyl, aryl, arylalkyl or —C(O)R⁵;
and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁸—CN, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(═N—CN)N(R⁴)R⁵, wherein each p is independently 0, 1, or 2 and each t is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
or R^{2a} and R^{2b}, together with the carbon ring atoms to which they are directly attached, may form a fused ring selected from cycloalkyl, aryl, heterocyclyl and heteroaryl, and R^{2c} and R^{2d} are as defined above;
or R^{2b} and R^{2c}, together with the carbon ring atoms to which they are directly attached, may form a fused ring selected from cycloalkyl, aryl, heterocyclyl and heteroaryl, and R^{2a} and R^{2d} are as defined above;
or R^{2c} and R^{2d}, together with the carbon ring atoms to which they are directly attached, may form a fused ring selected from cycloalkyl, aryl, heterocyclyl and heteroaryl, and R^{2a} and R^{2b} are as defined above;
each R³ is independently selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(N═C(R⁴)R⁵)N(R⁴)R⁵, wherein each p is independently 0, 1, or 2 and each t is independently 1 or 2;
and wherein each cycloalkyl, cycloalkylalkyl, aryl, aralkyl, aralkenyl, heterocyclyl, heterocyclylalkyl, heteroaryl and heteroarylalkyl for R³ may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
each R⁴ and R⁵ is independently selected from group consisting of hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl and heteroarylalkyl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain;
as a stereoisomer, enantiomer, tautomer thereof or mixtures thereof;
or a pharmaceutically acceptable salt, N-oxide, solvate or prodrug thereof.

48. The method of claim 47 wherein said disease or condition is selected from the group consisting of neuropathic pain, inflammatory pain, visceral pain, cancer pain, chemotherapy pain, trauma pain, surgical pain, post-surgical pain, childbirth pain, labor pain, neurogenic bladder, ulcerative colitis, chronic pain, persistent pain, peripherally mediated pain, centrally mediated pain, chronic headache, migraine headache, sinus headache, tension headache, phantom limb pain, peripheral nerve injury, and combinations thereof.

49. The method of claim 47, wherein said disease or condition is selected from the group consisting of pain associated with HIV, HIV treatment induced neuropathy, trigeminal neuralgia, post-herpetic neuralgia, eudynia, heat sensitivity, tosarcoidosis, irritable bowel syndrome, Crohns disease, pain associated with multiple sclerosis (MS), amyotrophic lateral sclerosis (ALS), diabetic neuropathy, peripheral neuropathy, arthritic, rheumatoid arthritis, osteoarthritis, atherosclerosis, paroxysmal dystonia, myasthenia syndromes, myotonia, malignant hyperthermia, cystic fibrosis, pseudoaldosteronism, rhabdomyolysis, hypothyroidism, bipolar depression, anxiety, schizophrenia, sodium channel toxin related illnesses, familial erythermalgia, primary erythermalgia, familial rectal pain, cancer, epilepsy, partial and general tonic seizures, restless leg syndrome, arrhythmias, fibromyalgia, neuroprotection under ischaemic conditions caused by stroke or neural trauma, tachy-arrhythmias, atrial fibrillation and ventricular fibrillation.

50. A method of treating pain in a mammal by the inhibition of ion flux through a voltage-dependent sodium channel in the mammal, wherein the method comprises administering to the mammal in need thereof a therapeutically effective amount of a compound of formula (I): 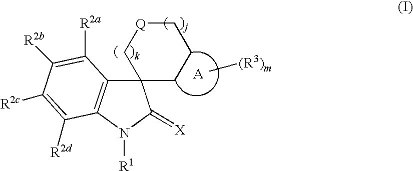
wherein:
j and k are each independently 0, 1, 2 or 3;
m is 0, 1, 2 or 4;
X is O or S; 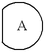
is a fused heterocyclyl ring or a fused heteroaryl ring;
Q is —C(R^{1a})₂—, —O—, —S(O)ₚ— (where p is 0, 1 or 2), —CF₂—, —OC(O)—, —C(O)O—, —C(O)N(R⁵)—, —N(R⁵)— or —N(R⁵)C(O)—;
each R^{1a} is hydrogen or —OR⁵;
or two R^{1a}'s, together with the carbon to which they are attached, form an oxo group;
R¹ is hydrogen, alkyl, alkenyl, alkynyl, haloalkyl, aryl, cycloalkyl, cycloalkylalkyl, heteroaryl, heterocyclyl, —R⁸—C(O)R⁵, —R⁸—C(O)OR⁵, —R⁸—C(O)N(R⁴)R⁵, —S(O)ₜ—R⁵ (where t is 1 or 2), —R⁹—S(O)ₚ—R⁵ (where p is 0, 1 or 2), —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
R⁶ is hydrogen, alkyl, aryl or aralkyl; and
R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
or R⁶ and R⁷, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by one or more substituents selected from the group consisting of —R⁸—OR⁵, —C(O)OR⁵, halo, haloalkyl, alkyl, nitro, cyano, aryl, aralkyl, heterocyclyl and heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
each R¹⁰ is hydrogen, alkyl, aryl, aralkyl or heteroaryl;
each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—N(R⁴)R⁵, —R⁹—OR⁵ or —R⁹—CN;
R¹² is hydrogen, alkyl, aryl, arylalkyl or —C(O)R⁵;
and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁸—CN, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(═N—CN)N(R⁴)R⁵, wherein each p is independently 0, 1, or 2 and each t is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
or R^{2a} and R^{2b}, together with the carbon ring atoms to which they are directly attached, may form a fused ring selected from cycloalkyl, aryl, heterocyclyl and heteroaryl, and R^{2c} and R^{2d} are as defined above;
or R^{2b} and R^{2c}, together with the carbon ring atoms to which they are directly attached, may form a fused ring selected from cycloalkyl, aryl, heterocyclyl and heteroaryl, and R^{2a} and R^{2d} are as defined above;
or R^{2c} and R^{2d}, together with the carbon ring atoms to which they are directly attached, may form a fused ring selected from cycloalkyl, aryl, heterocyclyl and heteroaryl, and R^{2a} and R^{2b} are as defined above;
each R³ is independently selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁸—CN, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(N═C(R⁴)R⁵)N(R⁴)R⁵, wherein each p is independently 0, 1, or 2 and each t is independently 1 or 2;
and wherein each cycloalkyl, cycloalkylalkyl, aryl, aralkyl, aralkenyl, heterocyclyl, heterocyclylalkyl, heteroaryl and heteroarylalkyl for R³ may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
each R⁴ and R⁵ is independently selected from group consisting of hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl and heteroarylalkyl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain;
as a stereoisomer, enantiomer, tautomer thereof or mixtures thereof;
or a pharmaceutically acceptable salt, N-oxide, solvate or prodrug thereof.

51. A method of decreasing ion flux through a voltage-dependent sodium channel in a cell in a mammal, wherein the method comprises contacting the cell with a compound of formula (I): 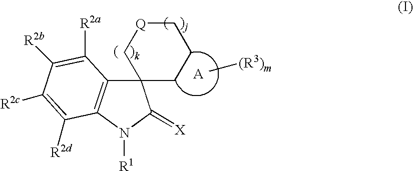
wherein:
j and k are each independently 0, 1, 2 or 3;
m is 0, 1, 2 or 4;
X is O or S; 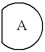
is a fused heterocyclyl ring or a fused heteroaryl ring;
Q is —C(R^{1a})₂—, —O—, —S(O)ₚ— (where p is 0, 1 or 2), —CF₂—, —OC(O)—, —C(O)O—, —C(O)N(R⁵)—, —N(R⁵)— or —N(R⁵)C(O)—;
each R^{1a} is hydrogen or —OR⁵;
or two R^{1a}'s, together with the carbon to which they are attached, form an oxo group;
R¹ is hydrogen, alkyl, alkenyl, alkynyl, haloalkyl, aryl, cycloalkyl, cycloalkylalkyl, heteroaryl, heterocyclyl, —R⁸—C(O)R⁵, —R⁸—C(O)OR⁵, —R⁸—C(O)N(R⁴)R⁵, —S(O)ₜ—R⁵ (where t is 1 or 2), —R⁹—S(O)ₚ—R⁵ (where p is 0, 1 or 2), —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
R⁶ is hydrogen, alkyl, aryl or aralkyl; and
R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
or R⁶ and R⁷, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—OR⁵, heterocyclyl and heteroaryl;
or R¹ is aralkyl optionally substituted by one or more substituents selected from the group consisting of —R⁸—OR⁵, —C(O)OR⁵, halo, haloalkyl, alkyl, nitro, cyano, aryl, aralkyl, heterocyclyl and heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
each R¹⁰ is hydrogen, alkyl, aryl, aralkyl or heteroaryl;
each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—N(R⁴)R⁵, —R⁹—OR⁵ or —R⁹—CN;
R¹² is hydrogen, alkyl, aryl, arylalkyl or —C(O)R⁵;
and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁸—CN, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(═N—CN)N(R⁴)R⁵, wherein each p is independently 0, 1, or 2 and each t is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
or R^{2a} and R^{2b}, together with the carbon ring atoms to which they are directly attached, may form a fused ring selected from cycloalkyl, aryl, heterocyclyl and heteroaryl, and R^{2c} and R^{2d} are as defined above;
or R^{2b} and R^{2c}, together with the carbon ring atoms to which they are directly attached, may form a fused ring selected from cycloalkyl, aryl, heterocyclyl and heteroaryl, and R^{2c} and R^{2d} are as defined above;
or R^{2c} and R^{2d}, together with the carbon ring atoms to which they are directly attached, may form a fused ring selected from cycloalkyl, aryl, heterocyclyl and heteroaryl, and R^{2a} and R^{2b} are as defined above;
each R³ is independently selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁸—CN, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(N═C(R⁴)R⁵)N(R⁴)R⁵, wherein each p is independently 0, 1, or 2 and each t is independently 1 or 2;
and wherein each cycloalkyl, cycloalkylalkyl, aryl, aralkyl, aralkenyl, heterocyclyl, heterocyclylalkyl, heteroaryl and heteroarylalkyl for R³ may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
each R⁴ and R⁵ is independently selected from group consisting of hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl and heteroarylalkyl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain;
as a stereoisomer, enantiomer, tautomer thereof or mixtures thereof;
or a pharmaceutically acceptable salt, N-oxide, solvate or prodrug thereof.

52. A method of treating hypercholesterolemia in a mammal, wherein the method comprises administering to the mammal in need thereof a therapeutically effective amount of a compound of formula (I): 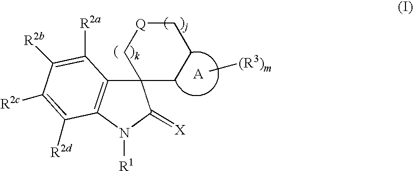
wherein:
j and k are each independently 0, 1, 2 or 3;
m is 0, 1, 2 or 4;
X is O or S; 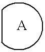
is a fused heterocyclyl ring or a fused heteroaryl ring;
Q is —C(R^{1a})₂—, —O—, —S(O)ₚ— (where p is 0, 1 or 2), —CF₂—, —OC(O)—, —C(O)O—, —C(O)N(R⁵)—, —N(R⁵)— or —N(R⁵)C(O)—;
each R^{1a} is hydrogen or —OR⁵;
or two R^{1a}'s, together with the carbon to which they are attached, form an oxo group;
R¹ is hydrogen, alkyl, alkenyl, alkynyl, haloalkyl, aryl, cycloalkyl, cycloalkylalkyl, heteroaryl, heterocyclyl, —R⁸—C(O)R⁵, —R⁸—C(O)OR⁵, —R⁸—C(O)N(R⁴)R⁵, —S(O)ₜ—R⁵ (where t is 1 or 2), —R⁹—S(O)ₚ—R⁵ (where p is 0, 1 or 2), —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
R⁶ is hydrogen, alkyl, aryl or aralkyl; and
R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
or R⁶ and R⁷, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—CN, —R⁸—OR⁵, heterocyclyl and heteroaryl;
or R¹ is aralkyl optionally substituted by one or more substituents selected from the group consisting of —R⁸—OR⁵, —C(O)OR⁵, halo, haloalkyl, alkyl, nitro, cyano, aryl, aralkyl, heterocyclyl and heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
each R¹⁰ is hydrogen, alkyl, aryl, aralkyl or heteroaryl;
each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—N(R⁴)R⁵, —R⁹—OR⁵ or —R⁹—CN;
R¹² is hydrogen, alkyl, aryl, arylalkyl or —C(O)R⁵;
and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁸—CN, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(═N—CN)N(R⁴)R⁵, wherein each p is independently 0, 1, or 2 and each t is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
or R^{2a} and R^{2b}, together with the carbon ring atoms to which they are directly attached, may form a fused ring selected from cycloalkyl, aryl, heterocyclyl and heteroaryl, and R^{2c} and R^{2d} are as defined above;
or R^{2b} and R^{2c}, together with the carbon ring atoms to which they are directly attached, may form a fused ring selected from cycloalkyl, aryl, heterocyclyl and heteroaryl, and R^{2a} and R^{2d} are as defined above;
or R^{2c} and R^{2d}, together with the carbon ring atoms to which they are directly attached, may form a fused ring selected from cycloalkyl, aryl, heterocyclyl and heteroaryl, and R^{2a} and R^{2b} are as defined above;
each R³ is independently selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(N═C(R⁴)R⁵)N(R⁴)R⁵, wherein each p is independently 0, 1, or 2 and each t is independently 1 or 2;
and wherein each cycloalkyl, cycloalkylalkyl, aryl, aralkyl, aralkenyl, heterocyclyl, heterocyclylalkyl, heteroaryl and heteroarylalkyl for R³ may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
each R⁴ and R⁵ is independently selected from group consisting of hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl and heteroarylalkyl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain;
as a stereoisomer, enantiomer, tautomer thereof or mixtures thereof;
or a pharmaceutically acceptable salt, N-oxide, solvate or prodrug thereof.

53. A method of treating benign prostatic hyperplasia in a mammal, wherein the method comprises administering to the mammal in need thereof a therapeutically effective amount of a compound of formula (I): 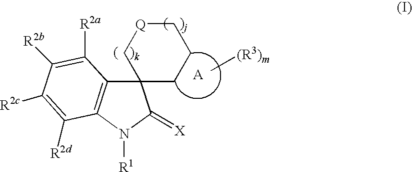
wherein:
j and k are each independently 0, 1, 2 or 3;
m is 0, 1, 2 or 4;
X is O or S; 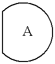
is a fused heterocyclyl ring or a fused heteroaryl ring;
Q is —C(R^{1a})₂—, —O—, —S(O)ₚ— (where p is 0, 1 or 2), —CF₂—, —OC(O)—, —C(O)O—, —C(O)N(R⁵)—, —N(R⁵)— or —N(R⁵)C(O)—;
each R^{1a} is hydrogen or —OR⁵;
or two R^{1a}'s, together with the carbon to which they are attached, form an oxo group;
R¹ is hydrogen, alkyl, alkenyl, alkynyl, haloalkyl, aryl, cycloalkyl, cycloalkylalkyl, heteroaryl, heterocyclyl, —R⁸—C(O)R⁵, —R⁸—C(O)OR⁵, —R⁸—C(O)N(R⁴)R⁵, —S(O)ₜ—R⁵ (where t is 1 or 2), —R⁹—S(O)ₚ—R⁵ (where p is 0, 1 or 2), —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
R⁶ is hydrogen, alkyl, aryl or aralkyl; and
R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
or R⁶ and R⁷, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, —R⁸—OR⁵, heterocyclyl and heteroaryl;
or R¹ is aralkyl optionally substituted by one or more substituents selected from the group consisting of —R⁸—OR⁵, —C(O)OR⁵, halo, haloalkyl, alkyl, nitro, cyano, aryl, aralkyl, heterocyclyl and heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
each R¹⁰ is hydrogen, alkyl, aryl, aralkyl or heteroaryl;
each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—N(R⁴)R⁵, —R⁹—OR⁵ or —R⁹—CN;
R¹² is hydrogen, alkyl, aryl, arylalkyl or —C(O)R⁵;
and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁸—CN, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(═N—CN)N(R⁴)R⁵, wherein each p is independently 0, 1, or 2 and each t is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
or R^{2a} and R^{2b}, together with the carbon ring atoms to which they are directly attached, may form a fused ring selected from cycloalkyl, aryl, heterocyclyl and heteroaryl, and R^{2c} and R^{2d} are as defined above;
or R^{2b} and R^{2c}, together with the carbon ring atoms to which they are directly attached, may form a fused ring selected from cycloalkyl, aryl, heterocyclyl and heteroaryl, and R^{2a} and R^{2d} are as defined above;
or R^{2c} and R^{2d}, together with the carbon ring atoms to which they are directly attached, may form a fused ring selected from cycloalkyl, aryl, heterocyclyl and heteroaryl, and R^{2a} and R^{2b} are as defined above;
each R³ is independently selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁸—CN, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(N═C(R⁴)R⁵)N(R⁴)R⁵, wherein each p is independently 0, 1, or 2 and each t is independently 1 or 2;
and wherein each cycloalkyl, cycloalkylalkyl, aryl, aralkyl, aralkenyl, heterocyclyl, heterocyclylalkyl, heteroaryl and heteroarylalkyl for R³ may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
each R⁴ and R⁵ is independently selected from group consisting of hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl and heteroarylalkyl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain;
as a stereoisomer, enantiomer, tautomer thereof or mixtures thereof;
or a pharmaceutically acceptable salt, N-oxide, solvate or prodrug thereof.

54. A method of treating treating pruritis in a mammal, wherein the method comprises administering to the mammal in need thereof a therapeutically effective amount of a compound of formula (I): 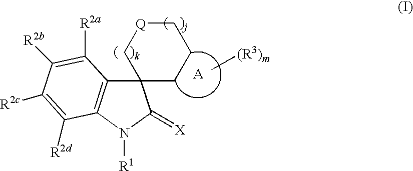
wherein:
j and k are each independently 0, 1, 2 or 3;
m is 0, 1, 2 or 4;
X is O or S; 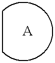
is a fused heterocyclyl ring or a fused heteroaryl ring;
Q is —C(R^{1a})₂—, —O—, —S(O)ₚ— (where p is 0, 1 or 2), —CF₂—, —OC(O)—, —C(O)O—, —C(O)N(R⁵)—, —N(R⁵)— or —N(R⁵)C(O)—;
each R^{1a} is hydrogen or —OR⁵;
or two R^{1a}'s, together with the carbon to which they are attached, form an oxo group;
R¹ is hydrogen, alkyl, alkenyl, alkynyl, haloalkyl, aryl, cycloalkyl, cycloalkylalkyl, heteroaryl, heterocyclyl, —R⁸—C(O)R⁵, —R⁸—C(O)OR⁵, —R⁸—C(O)N(R⁴)R⁵, —S(O)ₜ—R⁵ (where t is 1 or 2), —R⁹—S(O)ₚ—R⁵ (where p is 0, 1 or 2), —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
R⁶ is hydrogen, alkyl, aryl or aralkyl; and
R⁷ is hydrogen, alkyl, haloalkyl, —R⁹—CN, —R⁹—N(R⁴)R⁵, aryl, aralkyl, cycloalkyl, cycloalkylalkyl, heterocyclyl, heterocyclylalkyl, heteroaryl or heteroarylalkyl;
or R⁶ and R⁷, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, heterocyclyl and heteroaryl;
or R¹ is aralkyl optionally substituted by one or more substituents selected from the group consisting of —R⁸—OR⁵, —C(O)OR⁵, halo, haloalkyl, alkyl, nitro, cyano, aryl, aralkyl, heterocyclyl and heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
each R¹⁰ is hydrogen, alkyl, aryl, aralkyl or heteroaryl;
each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—N(R⁴)R⁵, —R⁹—OR⁵ or —R⁹—CN;
R¹² is hydrogen, alkyl, aryl, arylalkyl or —C(O)R⁵;
and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(═N—CN)N(R⁴)R⁵, wherein each p is independently 0, 1, or 2 and each t is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
or R^{2a} and R^{2b}, together with the carbon ring atoms to which they are directly attached, may form a fused ring selected from cycloalkyl, aryl, heterocyclyl and heteroaryl, and R^{2c} and R^{2d} are as defined above;
or R^{2b} and R^{2c}, together with the carbon ring atoms to which they are directly attached, may form a fused ring selected from cycloalkyl, aryl, heterocyclyl and heteroaryl, and R^{2a} and R^{2d} are as defined above;
or R^{2c} and R^{2d}, together with the carbon ring atoms to which they are directly attached, may form a fused ring selected from cycloalkyl, aryl, heterocyclyl and heteroaryl, and R^{2a} and R^{2b} are as defined above;
each R³ is independently selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁸—CN, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(N═C(R⁴)R⁵)N(R⁴)R⁵, wherein each p is independently 0, 1, or 2 and each t is independently 1 or 2;
and wherein each cycloalkyl, cycloalkylalkyl, aryl, aralkyl, aralkenyl, heterocyclyl, heterocyclylalkyl, heteroaryl and heteroarylalkyl for R³ may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
each R⁴ and R⁵ is independently selected from group consisting of hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl and heteroarylalkyl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain;
as a stereoisomer, enantiomer, tautomer thereof or mixtures thereof;
or a pharmaceutically acceptable salt, N-oxide, solvate or prodrug thereof.

55. A method of treating cancer in a mammal, wherein the methods comprise administering to the mammal in need thereof a therapeutically effective amount of a compound of formula (I): 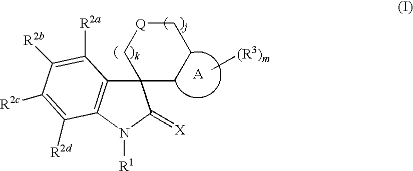
wherein:
j and k are each independently 0, 1, 2 or 3;
m is 0, 1, 2 or 4;
X is O or S; 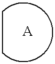
is a fused heterocyclyl ring or a fused heteroaryl ring;
Q is —C(R^{1a})₂—, —O—, —S(O)ₚ— (where p is 0, 1 or 2), —CF₂—, —OC(O)—, —C(O)O—, —C(O)N(R⁵)—, —N(R⁵)— or —N(R⁵)C(O)—;
each R^{1a} is hydrogen or —OR⁵;
or two R^{1a}'s, together with the carbon to which they are attached, form an oxo group;
R¹ is hydrogen, alkyl, alkenyl, alkynyl, haloalkyl, aryl, cycloalkyl, cycloalkylalkyl, heteroaryl, heterocyclyl, —R⁸—C(O)R⁵, —R⁸—C(O)OR⁵, —R⁸—C(O)N(R⁴)R⁵, —S(O)ₜ—R⁵ (where t is 1 or 2), —R⁹—S(O)ₚ—R⁵ (where p is 0, 1 or 2), —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
R⁶ is hydrogen, alkyl, aryl or aralkyl; and
R⁷ is hydrogen, alkyl, haloalkyl, —R⁹—CN, —R⁹—OR⁵, —R⁹—N(R⁴)R⁵, aryl, aralkyl, cycloalkyl, cycloalkylalkyl, heterocyclyl, heterocyclylalkyl, heteroaryl or heteroarylalkyl;
or R⁶ and R⁷, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, heterocyclyl and heteroaryl;
or R¹ is aralkyl optionally substituted by one or more substituents selected from the group consisting of —R⁸—OR⁵, —C(O)OR⁵, halo, haloalkyl, alkyl, nitro, cyano, aryl, aralkyl, heterocyclyl and heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
each R¹⁰ is hydrogen, alkyl, aryl, aralkyl or heteroaryl;
each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—N(R⁴)R⁵, —R⁹—OR⁵ or —R⁹—CN;
R¹² is hydrogen, alkyl, aryl, arylalkyl or —C(O)R⁵;
and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(═N—CN)N(R⁴)R⁵, wherein each p is independently 0, 1, or 2 and each t is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
or R^{2a} and R^{2b}, together with the carbon ring atoms to which they are directly attached, may form a fused ring selected from cycloalkyl, aryl, heterocyclyl and heteroaryl, and R^{2c} and R^{2d} are as defined above;
or R^{2b} and R^{2c}, together with the carbon ring atoms to which they are directly attached, may form a fused ring selected from cycloalkyl, aryl, heterocyclyl and heteroaryl, and R^{2a} and R^{2d} are as defined above;
or R^{2c} and R^{2d}, together with the carbon ring atoms to which they are directly attached, may form a fused ring selected from cycloalkyl, aryl, heterocyclyl and heteroaryl, and R^{2a} and R^{2b} are as defined above;
each R³ is independently selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁸—CN, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(N═C(R⁴)R⁵)N(R⁴)R⁵, wherein each p is independently 0, 1, or 2 and each t is independently 1 or 2;
and wherein each cycloalkyl, cycloalkylalkyl, aryl, aralkyl, aralkenyl, heterocyclyl, heterocyclylalkyl, heteroaryl and heteroarylalkyl for R³ may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
each R⁴ and R⁵ is independently selected from group consisting of hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl and heteroarylalkyl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain;
as a stereoisomer, enantiomer, tautomer thereof or mixtures thereof;
or a pharmaceutically acceptable salt, N-oxide, solvate or prodrug thereof.

56. A pharmaceutical composition comprising a pharmaceutically acceptable excipient and a compound of formula (I): 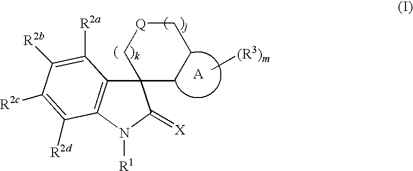
wherein:
j and k are each independently 0, 1, 2 or 3;
m is 0, 1, 2 or 4;
X is O or S; 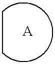
is a fused heterocyclyl ring or a fused heteroaryl ring;
Q is —C(R^{1a})₂—, —O—, —S(O)ₚ— (where p is 0, 1 or 2), —CF₂—, —OC(O)—, —C(O)O—,
—C(O)N(R⁵)—, —N(R⁵)— or —N(R⁵)C(O)—;
each R^{1a} is hydrogen or —OR⁵;
or two R^{1a}'s, together with the carbon to which they are attached, form an oxo group;
R¹ is hydrogen, alkyl, alkenyl, alkynyl, haloalkyl, aryl, cycloalkyl, cycloalkylalkyl, heteroaryl, heterocyclyl, —R⁸—C(O)R⁵, —R⁸—C(O)OR⁵, —R⁸—C(O)N(R⁴)R⁵, —S(O)ₜ—R⁵ (where t is 1 or 2), —R⁹—S(O)ₚ—R⁵ (where p is 0, 1 or 2), —R⁸—OR⁵, —R⁸—CN, —R⁹—P(O)(OR⁵)₂ or —R⁹—O—R⁹—OR⁵;
or R¹ is aralkyl substituted by —C(O)N(R⁶)R⁷ where:
R⁶ is hydrogen, alkyl, aryl or aralkyl; and
R⁷ is hydrogen, alkyl, haloalkyl, —R⁹—CN, —R⁹—N(R⁴)R⁵, aryl, aralkyl, cycloalkyl, cycloalkylalkyl, heterocyclyl, heterocyclylalkyl, heteroaryl or heteroarylalkyl;
or R⁶ and R⁷, together with the nitrogen to which they are attached, form a heterocyclyl or heteroaryl;
and wherein each aryl, aralkyl, cycloalkyl, cycloalkylalkyl, heterocyclyl, heterocyclylalkyl, heteroaryl and heteroaryl groups for R⁶ and R⁷ may be optionally substituted by one or more substituents selected from the group consisting of alkyl, cycloalkyl, aryl, aralkyl, halo, haloalkyl, heterocyclyl and heteroaryl;
or R¹ is aralkyl optionally substituted by one or more substituents selected from the group consisting of —R⁸—OR⁵, —C(O)OR⁵, halo, haloalkyl, alkyl, nitro, cyano, aryl, aralkyl, heterocyclyl and heteroaryl;
or R¹ is —R⁹—N(R¹⁰)R¹¹, —R⁹—N(R¹²)C(O)R¹¹, —R⁹—C(O)N(R¹²)R¹¹ or —R⁹—N(R¹⁰)C(O)N(R¹⁰)R¹¹ where:
each R¹⁰ is hydrogen, alkyl, aryl, aralkyl or heteroaryl;
each R¹¹ is hydrogen, alkyl, haloalkyl, cycloalkyl, cycloalkylalkyl, aryl, aralkyl, heterocyclyl, heterocyclylalkyl, heteroaryl, heteroarylalkyl, —R⁹—OC(O)R⁵, —R⁹—C(O)OR⁵, —R⁹—C(O)N(R⁴)R⁵, —R⁹—C(O)R⁵, —R⁹—N(R⁴)R⁵, —R⁹—OR⁵ or —R⁹—CN;
R¹² is hydrogen, alkyl, aryl, arylalkyl or —C(O)R⁵;
and wherein each aryl, aralkyl, cycloalkyl, cycloalkylalkyl, heterocyclyl, heterocycylalkyl, heteroaryl and heteroarylalkyl groups for R¹⁰ and R¹¹ may be optionally substituted by one or more substituents selected from the group consisting of alkyl, cycloalkyl, aryl, aralkyl, halo, haloalkyl, nitro, —R⁸—CN, —R⁸—OR⁵, —R⁸—C(O)R⁵, heterocyclyl and heteroaryl;
or R¹ is heterocyclylalkyl or heteroarylalkyl where the heterocyclylalkyl or the heteroarylalkyl group is optionally substituted by one or more substituents selected from the group consisting of alkyl, halo, haloalkyl, —R⁸—OR⁵, —R⁸—C(O)OR⁵, aryl and aralkyl;
R^{2a}, R^{2b}, R^{2c} and R^{2d} are each independently selected from the group consisting of hydrogen, alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, —R⁸—CN, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(═N—CN)N(R⁴)R⁵, wherein each p is independently 0, 1, or 2 and each t is independently 1 or 2;
and wherein each of the cycloalkyl, cycloalkylalkyl, aryl, aralkyl, aralkenyl, heterocyclyl, heterocyclylalkyl, heteroaryl and heteroarylalkyl groups for R^{2a}, R^{2b}, R^{2c} and R^{2d} may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
or R^{2a} and R^{2b}, together with the carbon ring atoms to which they are directly attached, may form a fused ring selected from cycloalkyl, aryl, heterocyclyl and heteroaryl, and R^{2a} and R^{2d} are as defined above;
or R^{2b} and R^{2c}, together with the carbon ring atoms to which they are directly attached, may form a fused ring selected from cycloalkyl, aryl, heterocyclyl and heteroaryl, and R^{2a} and R^{2d} are as defined above;
or R^{2c} and R^{2d}, together with the carbon ring atoms to which they are directly attached, may form a fused ring selected from cycloalkyl, aryl, heterocyclyl and heteroaryl, and R^{2a} and R^{2b} are as defined above;
each R³ is independently selected from the group consisting of alkyl, alkenyl, alkynyl, halo, haloalkyl, haloalkenyl, cycloalkyl, cycloalkylalkyl, aryl, aralkyl, aralkenyl, heterocyclyl, heterocyclylalkyl, heteroaryl, heteroarylalkyl, oxo, —R⁸—CN, —R⁸—NO₂, —R⁸—OR⁵, —R⁸—N(R⁴)R⁵, —R⁸—N═C(R⁴)R⁵, —R⁸—S(O)ₚR⁴, —R⁸—OS(O)₂CF₃, —R⁸—C(O)R⁴, —R⁸—C(S)R⁴, —R⁸—C(O)OR⁴, —R⁸—C(S)OR⁴, —R⁸—C(O)N(R⁴)R⁵, —R⁸—C(S)N(R⁴)R⁵, —R⁸—N(R⁵)C(O)R⁴, —R⁸—N(R⁵)C(S)R⁴, —R⁸—N(R⁵)C(O)OR⁴, —R⁸—N(R⁵)C(S)OR⁴, —R⁸—N(R⁵)C(O)N(R⁴)R⁵, —R⁸—N(R⁵)C(S)N(R⁴)R⁵, —R⁸—N(R⁵)S(O)ₜR⁴, —R⁸—N(R⁵)S(O)ₜN(R⁴)R⁵, —R⁸—S(O)ₜN(R⁴)R⁵, —R⁸—N(R⁵)C(═NR⁵)N(R⁴)R⁵ and —R⁸—N(R⁵)C(N═C(R⁴)R⁵)N(R⁴)R⁵, wherein each p is independently 0, 1, or 2 and each t is independently 1 or 2;
and wherein each cycloalkyl, cycloalkylalkyl, aryl, aralkyl, aralkenyl, heterocyclyl, heterocyclylalkyl, heteroaryl and heteroarylalkyl for R³ may be optionally substituted by one or more substituents selected from the group consisting of alkyl, alkenyl, alkynyl, alkoxy, halo, haloalkyl, haloalkenyl, haloalkoxy, cycloalkyl, cycloalkylalkyl, aryl, aralkyl, aralkenyl, heterocyclyl, heterocyclylalkyl, heteroaryl, heteroarylalkyl, —R⁸—CN, —R⁸—N(O)₂, —R⁸—OR⁵, —R⁸—N(R⁴)R⁵, —S(O)ₚR⁴, —R⁸—C(O)R⁴, —R⁸—C(O)OR⁴, —R⁸—C(O)N(R⁴)R⁵, —N(R⁵)C(O)R⁴ and —N(R⁵)S(O)ₜR⁴, wherein each p is independently 0, 1, or 2 and each t is independently 1 or 2;
each R⁴ and R⁵ is independently selected from group consisting of hydrogen, alkyl, alkenyl, alkynyl, haloalkyl, haloalkenyl, alkoxyalkyl, cycloalkyl, cycloalkylalkyl, aryl, aralkyl, heterocyclyl, heterocyclylalkyl, heteroaryl and heteroarylalkyl;
or when R⁴ and R⁵ are each attached to the same nitrogen atom, then R⁴ and R⁵, together with the nitrogen atom to which they are attached, may form a heterocyclyl or heteroaryl; and
each R⁸ is a direct bond or a straight or branched alkylene chain, a straight or branched alkenylene chain or a straight or branched alkynylene chain; and
each R⁹ is a straight or branched alkylene chain, a straight or branched alkenylene chain or a straight or branched alkynylene chain;
as a stereoisomer, enantiomer, tautomer thereof or mixtures thereof;
or a pharmaceutically acceptable salt, N-oxide, solvate or prodrug thereof.
